(19) United States
(12) Patent Application Publication
(10) Pub. No.: US 2015/0209477 A1
(43) Pub. Date: Jul. 30, 2015

(54) COMPOSITIONS FOR BIOMEDICAL APPLICATIONS

(71) Applicant: University of Massachusetts Medical School, Boston, MA (US)

(72) Inventors: Jane B. Lian, Westborough, MA (US); Xinning Li, Framingham, MA (US); David C. Ayers, Worcester, MA (US); Jie Song, Shrewsbury, MA (US)

(21) Appl. No.: 14/541,401

(22) Filed: Nov. 14, 2014

Related U.S. Application Data
(62) Division of application No. 12/183,293, filed on Jul. 31, 2008.
(60) Provisional application No. 60/963,336, filed on Aug. 3, 2007.

(51) Int. Cl. A61L 27/46; A61L 27/54; A61L 27/56; A61L 27/38

(52) U.S. Cl. CPC A61L 27/46; A61L 27/3804; A61L 27/54; A61L 27/56; A61L 2430/02

(57) ABSTRACT

The invention relates to composite materials that contain a polymer matrix and aggregates, and in some embodiments, methods of making, and methods of using these materials. Preferably, the aggregates are calcium phosphate aggregates. Preferably, the material is resistant to fracture. In further embodiments, the materials are used in surgical procedures of bone replacement. In further embodiments, the materials contain polyhedral silsesquioxanes and/or biodegradable segments. In further embodiments, the polymer matrix comprises biomolecules.

### STATEMENT OF GOVERNMENT SUPPORT

This invention was made in part with government support under grant number 1R01AR055615-01, from the National Institutes of Health. As such, the United States government has certain rights to the invention.

### FIELD OF INVENTION

The invention relates to composite materials that contain a polymer matrix and aggregates, and in some embodiments, methods of making and methods of using these materials. Preferably, the aggregates are calcium phosphate aggregates. Preferably, the materials are resistant to fracture. In further embodiments, the materials are used in surgical procedures of bone and joint replacement.

### BACKGROUND

Surgical removal of bone segments is a common treatment with a diagnosis of osteosarcoma. The lack of a bone segment presents substantial problems for the patients, which are typically addressed by bone grafts. Bone cement such as Plexiglass, polymethylmethacrylate (PMMA), is used in joint, hip and shoulder replacement surgeries to bond metallic devices with bone. The benefits of such surgeries suffer from a relatively short lifetime due to PMMA's limited capacity to integrate with bony tissue. Other porous and biodegradable scaffolds are generally not suitable for load bearing applications since they are weak and susceptible to fatigue and fracture. Thus, there is a compelling need to develop bone substitutes that provide flexibility to facilitate surgical fitting that do not initiate immunological responses and allow for biointegration and biodegradation during the healing process.

### SUMMARY OF INVENTION

The invention relates to composite materials that contain a polymer matrix and aggregates, and in some embodiments, methods of making and methods of using these materials. Preferably, the aggregates are calcium phosphate aggregates. Preferably, the materials are resistant to fracture. In further embodiments, the materials are used in surgical procedures of bone and joint replacement.

In some embodiments, the invention relates to a method comprising: providing a bone and composite materials disclosed herein and connecting said bone with said composite material. In further embodiments, said bone is cortical bone or cancellous bone. In further embodiments, said bone is a mandible. In further embodiments, said bone is located in an animal. It is not intended that the present invention be limited by the nature of the bone or the bone's location in the body. A plurality of bone types is contemplated. In further embodiments, said bone is cortical bone or cancellous bone. In further embodiments, said bone is a mandible. In further embodiments, said bone is located in an animal. In further embodiments, said bone is in or near a jaw, joint, hip, shoulder, elbow, pelvis or ankle. In further embodiments, the skin of said animal covers said composite material. In further embodiments, said composite material degrades and new bone forms in its place.

In some embodiments, the invention relates to a siloxane macromer comprising polymer arms comprising a polymer segment comprising: a) monomers comprising hydroxyl groups, b) a reactive group configured to crosslink said siloxane macromer, and c) a connecting group configured to covalently link a biomolecule. In further embodiments, said polymer arms comprise a second polymer segment comprising polylactone. In further embodiments, said reactive group and connecting group are is selected from the group consisting of hydroxyl, amine, carboxylate, epoxy, azido, methacrylate, methacrylamide, acrylate, acrylamide, alkoxysilane, alkynyl, vinyl, isocyanate, azido, ethynyl, trithiocarbonate, and dithioester groups.

In some embodiments, the invention relates to a polymer matrix comprising: a) a polymer comprising siloxane macromers, wherein said siloxane macromers comprise polymer arms comprising a polymer segment comprising monomers comprising hydroxyl groups and a connecting group, and b) cross-linkers covalently linking said monomer siloxane macromers. In further embodiments, said cross-linkers comprise polyethylene glycol subunits or alkyl. In further embodiments, said polymer comprises a biomolecule covalently linked through said connecting group. In further embodiments, said biomolecule is selected from the group consisting of a bone mineral binding peptide, an intigrin binding peptide, anionic or cationic motifs that binds oppositely charged second biomolecule, ligand that binds a second biomolecule. In further embodiments, said second biomolecule is selected from the group consisting of proteins, growth factors, cytokines, recombinant proteins, and gene vectors. In further embodiments, said siloxane is selected from the group consisting of silsesquioxanes and metallasiloxanes. In further embodiments, said siloxane is a caged structure. In further embodiments, said siloxane is a polyhedral silsesquioxane. In further embodiments, said siloxane is octakis(hydridodimethylsiloxy)octasesquioxane. In further embodiments, said siloxane macromer is a siloxane substituted with a polylactone. In further embodiments, said siloxane macromer is POSS-(PLAₙ-co-pHEMAₘ)₁₋₈ or POSS-(PLAₙ)₁₋₈ wherein n is 3 to 200 and m is 3 to 1000.

In some embodiments, the invention relates to a composite material comprising the polymer matrix and aggregates distributed within said polymer matrix. In further embodiments, said material is biodegradable. In further embodiments, said aggregates are selected from the group consisting of calcium hydroxyapatite, and carbonated hydroxyapatite, and beta-tricalcium phosphate.

In some embodiments, the invention relates to a method of making a composite material comprising: i) providing: a) aggregates, b) a siloxane macromer comprising polymer arms comprising a polymer segment comprising: i) monomers comprising hydroxyl groups, ii) a reactive group configured to crosslink said siloxane macromer, and iii) a connecting group configured to covalently link a biomolecule, c) a cross-linker, and d) a solvent; and ii) mixing said calcium phosphate aggregates with said siloxane macromer and cross-linker in said solvent under conditions such that a composite material is formed. In further embodiments, said siloxane macromer comprises a biomolecule covalently linked through said connecting group. In further embodiments, said polymer comprises a biomolecule covalently linked through said connecting group. In further embodiments, said biomolecule is selected from the group consisting of a bone mineral binding peptide, an intigrin binding peptide, anionic or cationic motifs that binds oppositely charged second biomolecule, ligand that binds a second biomolecule. In further embodiments, said second biomolecule is selected from the group consisting of proteins, growth factors, cytokines, recombinant proteins, and gene vectors. In further embodiments, said solvent further comprises a radical initiator. In further embodiments, said radical initiator is hydrophilic. In further embodiments, said radical initiator is selected form the group consisting of ammonium persulfate and sodium metasulfite. In further embodiments, said reactive groups are selected from the group consisting of hydroxyl, amine, carboxylate, epoxy, azido, methacrylate, methacrylamide, acrylate, acrylamide, alkoxysilane, alkynl, vinyl, isocyanate, azido, ethynyl, trithiocarbonate and dithioester groups. In further embodiments, said cross-linker further comprises ethylene glycol subunits. In further embodiments, said solvent is a hydrophilic solvent. In further embodiments, more than half of said hydrophilic solvent by volume comprises molecules selected from the group consisting of water, ethylene glycol and polyethylene glycol. In further embodiments, said siloxane macromer comprises a polyhedral silsesquioxane. In further embodiments, said siloxane macromer comprises octakis (hydridodimethylsiloxy) octasesquioxane. In further embodiments, said cross-linker is a diisocyanate cross-linker.

In some embodiments, the invention relates to dental applications such as artificial teeth that comprise composites disclosed herein.

In some embodiments, the invention relates to a siloxane macromer comprising polymer arms comprising a polymer segment comprising hydroxyl groups and a reactive group configured to crosslink the siloxane macromer. In further embodiments, said polymer arms comprise a second polymer segment comprising polylactone. In further embodiments, said reactive group is selected from the group consisting of hydroxyl, amine, carboxylate, epoxy, azido, methacrylate, methacrylamide, acrylate, acrylamide, alkoxysilane, alkynyl vinyl, isocyanate, azido, ethynyl, trithiocarbonate, and dithioester groups. In further embodiments, said reactive groups are configured to covalently link bioactive molecules.

In some embodiments, the invention relates to a polymer matrix comprising: a) a polymer comprising monomer siloxane macromers covalently linked, wherein said siloxane macromers comprise polymer arms comprising a polymer segment comprising hydroxyl groups and a connecting group, and b) cross-linkers covalently linking said monomer siloxane macromers through said connecting group. In further embodiments, said cross-linkers comprise polyethylene glycol subunits or alkyl. In further embodiments, said polymer comprises a biomolecule covalently linked through said connecting group. In further embodiments, said biomolecule is selected from the group consisting of a bone mineral binding peptide, an intigrin binding peptide, anionic or cationic motifs that binds oppositely charged second biomolecule. In further embodiments, said second biomolecule is selected from the group consisting of proteins, growth factors, cytokines, recombinant proteins, and gene vectors. In further embodiments, said siloxane is selected from the group consisting of silsesquioxanes and metallasiloxanes. In further embodiments, said siloxane is a caged structure. In further embodiments, said siloxane is a polyhedral silsesquioxane. In further embodiments, said siloxane is octakis (hydridodimethylsiloxy)octasesquioxane. In further embodiments, said siloxane macromer is a siloxane substituted with a polylactone. In further embodiments, said siloxane macromer comprises POSS-(PLAₙ-co-pHEMAₘ)₁₋₈ or POSS-(PLAₙ)₁₋₈ wherein n is 3 to 200 and m is 3 to 1000.

In further embodiments, the invention relates to a composite material comprising the polymer matrix and calcium phosphate aggregates distributed within said polymer matrix. In further embodiments, said material is biodegradable. In further embodiments, said calcium phosphate aggregates are selected from the group consisting of calcium hydroxyapatite, and carbonated hydroxyapatite, and beta-tricalcium phosphate.

In some embodiments, the invention relates to method of making a composite material comprising: i) providing: a) calcium phosphate aggregates, b) a siloxane macromer comprising polymer anus comprising a polymer segment comprising hydroxyl groups and a reactive group, c) a cross-linker, and d) a solvent; and ii) mixing said calcium phosphate aggregates with said siloxane macromer and cross-linker in said solvent under conditions such that a composite material is formed. In further embodiments, said cross-linker is a diisocyanate cross-linker.

In some embodiments the invention relates to a composite material comprising: a) a polymer matrix comprising a polymer comprising monomer subunits comprising hydroxyl groups, wherein said monomers are linked via a covalent linkage comprising polyethylene glycol subunits; b) calcium phosphate aggregates distributed within said polymer matrix; and c) a biomolecule. In further embodiments, said biomolecule is selected from the group comprising peptides, saccharides, and nucleic acids. In further embodiments, said biomolecules are growth factors. In further embodiments, said biomolecules are selected from the group consisting of BMP-2, BMP-2/7 heterodiamer, RANKL, and VEGF. In further embodiments, said biomolecules comprise a peptide with SEQ ID No.:1 or SEQ ID No.:2. In further embodiments, said peptide can be at least 70% homologous, more preferably at least 80% homologous, still more preferably at least 90% homologous and most preferably 95% homologous or more to sequences disclosed herein or known sequences. In further embodiments, said nucleic acid is a gene vector. In further embodiments, said gene vector encodes a protein selected from the group consisting of BMP-2, BMP-2/7 heterodiamer, RANKL, and VEGF.

In some embodiments the invention relates to a composite material comprising: a) a polymer matrix comprising a polymer comprising monomer subunits comprising hydroxyl groups, wherein said monomers are linked via a covalent linkage comprising polyethylene glycol subunits; b) calcium phosphate aggregates distributed within said polymer matrix; and c) one or more antibiotics. In a preferred embodiment, the antibiotic is a broad spectrum antibiotic, such as tetracycline.

In some embodiments the invention relates to a composite material comprising: a) a polymer matrix comprising a polymer comprising monomer subunits comprising hydroxyl groups, wherein said monomers are linked via a covalent linkage comprising polyethylene glycol subunits; b) calcium phosphate aggregates distributed within said polymer matrix; and c) stem cells (or osteoblast precursor cells). In a preferred embodiments, said stem cells are bone marrow stem cells. In further embodiments, said stem cells are contacted by exogenously added growth factors. In one embodiment, osteoblast precursor cells are seeded into the matrix under conditions such that they differentiate into osteoblasts. In preferred embodiment, the cells are incubated at 37° C. in humidified environment with 5% CO₂ without additional media for 2 to 24 hrs (preferably 6 hrs) to allow cell attachment.

In further embodiments, said material is elastic. In further embodiments, said material does not fracture under a compression of force between 29 and 100 MPa. In further embodiments, said monomer subunits are substituted or unsubstituted hydroxyalkyl acrylate subunits. In further embodiments, said monomer subunits are 2-hydroxyethyl methacrylate subunits. In further embodiments, said calcium phosphate aggregates are selected from the group consisting of calcium hydroxyapatite and beta-tricalcium phosphate. In further embodiments, said material is between 10%-90% by weight calcium phosphate aggregates distributed within said polymer matrix.

In some embodiments, the invention relates to a porous composite material comprising: a) a polymer matrix comprising a polymer comprising monomer subunits comprising hydroxyl groups, wherein said monomers are linked via a covalent linkage comprising polyethylene glycol subunits; b) calcium phosphate aggregates distributed within said polymer matrix; and c) cells. In further embodiments, the composite comprises a biomolecule selected from the group consisting of growth factors, cytokines, gene vectors, and retroviruses. In further embodiments, said pores are greater than 1 millimeter in diameter. In further embodiments, said pores are as large as 90%, 50%, 30%, 10%, 5% of the outer diameter of the material. In further embodiments, said pores are as small as 1%, 0.1%, 0.01%, or 0.001% of the outer diameter of the material. In further embodiments, said pores are between 100 micrometer and 5 millimeter in diameter.

In some embodiments, the invention relates to a method comprising: a) providing a composite material comprising: i) a polymer matrix comprising a polymer comprising monomer subunits comprising hydroxyl groups, wherein said monomers are linked via a covalent linkage comprising polyethylene glycol subunits, and ii) calcium phosphate aggregates distributed within said polymer matrix; b) creating pores in said composite material providing a porous material; c) mixing said porous material with a component selected from the group consisting of cells and biomolecules providing a loaded composite; and d) implanting said loaded composite into a subject. In further embodiments, said cells are bone marrow cells. In further embodiments, said biomolecule is selected from the group consisting of growth factors, cytokines, gene vectors, and retroviruses. In further embodiments, said subject is a selected from the group consisting of a human, mouse, rat, dog, cat, rabbit, pig, horse, and ape.

In some embodiments, the invention relates to a material composition made by a) providing, i) a polymer matrix comprising: A) a polymer comprising 2-hydroxyethyl methacrylate subunits, wherein said monomers are linked via a covalent linkage comprising polyethylene glycol subunits, B) calcium phosphate aggregates distributed within said polymer matrix; and ii) a biomolecule; b) mixing said polymer matrix and said biomolecule under conditions such that said biomolecule is absorbed to said material.

In some embodiments, the invention relates to a composite material comprising: a) a polymer matrix comprising a polymer comprising monomers of 2-hydroxyethyl methacrylate subunits, wherein said monomers are linked via a covalent linkage comprising polyethylene glycol subunits; b) calcium phosphate aggregates distributed within said polymer matrix; and c) a peptide.

In some embodiments, the invention relates to a polymer matrix comprising: a) a polymer comprising monomer subunits comprising hydroxyl groups, wherein said monomers are linked via a covalent linkage, and b) a siloxane covalently attached to said polymer matrix. In further embodiments said siloxane macromer comprises a covalently linked peptide.

In further embodiments, the invention relates to a composite material comprising: a) a polymer matrix comprising: i) a polymer comprising monomer subunits comprising hydroxyl groups, wherein said monomers are linked via a covalent linkage, and ii) a siloxane covalently attached to said polymer matrix; and b) calcium phosphate aggregates distributed within said polymer matrix. In further embodiments, said siloxane is a siloxane macromer. In further embodiments, said material is biodegradable. In further embodiments, said siloxane macromer is POSS-(PLAₙ-co-pHEMAₘ)₁₋₈ or POSS-(PLAₙ)₁₋₈ wherein n is 3 to 40 and m is 3 to 1000.

In further embodiments, the invention relates to a method of making a composite material comprising: i) providing: a) calcium phosphate aggregates, b) monomers comprising a first reactive group and a hydroxyl group, c) a cross-linker comprising a siloxane comprising two or more reactive groups, and d) a hydrophilic solvent; and ii) mixing said calcium phosphate aggregates with said monomers and cross-linker in said solvent under conditions such that a composite material is formed.

In some embodiments, the invention relates to a composite material comprising: a) a polymer matrix comprising: i) a polymer comprising monomer subunits comprising hydroxyl groups and ii) a cross-linker comprising polyethylene glycol subunits; b) calcium phosphate aggregates distributed within said polymer matrix; and c) a biomolecule. In further embodiments, said biomolecule promotes osteogenesis. In further embodiments, said biomolecules are peptides. In further embodiments, said biomolecules are cell adhesive ligands or mineral nucleating ligands. In further embodiments, said biomolecules comprise peptide SEQ ID NO.: 1 or SEQ ID NO.: 2. In further embodiments, said biomolecules are growth factors. In further embodiments, said biomolecules are selected from the group consisting of rhBMP-2, rmRANKL, and rhVEGF165. In further embodiments, said material is elastic. In further embodiments, said material does not fracture under a compression of force between 29 and 100 MPa. In further embodiments, said monomer subunits are substituted or unsubstituted hydroxyalkyl acrylate subunits. In further embodiments, monomer subunits are 2-hydroxyethyl methacrylate subunits. In further embodiments, said calcium phosphate aggregates are selected from the group consisting of calcium hydroxyapatite and beta-tricalcium phosphate. In further embodiments, said material is between 10%-90% by weight calcium phosphate aggregates distributed within said polymer matrix.

In some embodiments, the invention relates to a polymer matrix comprising: a) a polymer comprising monomer subunits comprising hydroxyl groups, b) cross-linkers, and c) siloxane macromers covalently attached to said polymer matrix. In further embodiments, said cross-linkers comprise polyethylene glycol subunits. In further embodiments, said siloxane macromers are second cross-linkers. In further embodiments, said siloxane macromers comprise covalently attached biomolecules. In further embodiments, said biomolecule is a calcium phosphate binding peptide. In further embodiments, said siloxane is selected from the group consisting of silsesquioxanes and metallasiloxanes. In further embodiments, said siloxane is a caged structure. In further embodiments, said siloxane is a polyhedral silsesquioxane. In further embodiments, said siloxane is octakis(hydridodimethylsiloxy)octasesquioxane. In further embodiments, said siloxane macromer is a siloxane substituted with a polylactone. In further embodiments, said siloxane macromer is a siloxane substituted with a polylactide.

In further embodiments, the invention relates to a composite material comprising a polymer matrix disclosed herein and calcium phosphate aggregates distributed within said polymer matrix. In further embodiments, said material is biodegradable.

In some embodiments, the invention relates to a material composition made by a) providing, i) a polymer matrix comprising: A) a polymer comprising 2-hydroxyethyl methacrylate subunits, B) a cross-linker comprising polyethylene glycol subunits, C) calcium phosphate aggregates distributed within said polymer matrix; and ii) a biomolecule; b) mixing said polymer matrix and said biomolecule under conditions such that said biomolecule is absorbed to said material. In further embodiments, said calcium phosphate aggregates are selected from the group consisting of calcium hydroxyapatite and beta-tricalcium phosphate aggregates. In further embodiments, said calcium phosphate aggregates have a size between 50 nanometers and 50 micrometers. In further embodiments, said calcium phosphate aggregates are between 30%-70% by weight of said material. In further embodiments, said calcium phosphate aggregates are between 10%-90% by weight of said material.

In some embodiments, the invention relates to a composite material comprising: a) a polymer matrix comprising: i) a polymer comprising monomers of 2-hydroxyethyl methacrylate subunits and ii) a cross-linker comprising polyethylene glycol subunits; b) calcium phosphate aggregates distributed within said polymer matrix; and c) a peptide.

In some embodiments, the invention relates to a method of making a composite material comprising: i) providing: a) calcium phosphate aggregates, b) monomers comprising a first reactive group and a hydroxyl group, c) hydrophilic cross-linkers comprising two or more reactive groups, and d) a hydrophilic solvent; and ii) mixing said calcium phosphate aggregates, monomers and cross-linkers in said solvent under conditions such that a composite material is formed. In further embodiments, said solution further comprises a radical initiator. In further embodiments, said radical initiator is hydrophilic. In further embodiments, said radical initiator is selected from the group consisting of ammonium persulfate and sodium metasulfite. In further embodiments, said reactive groups are selected from the group consisting of vinyl, isocyanate, azido, ethynyl, trithiocarbonate and dithioester groups. In further embodiments, said first reactive group is a vinyl group. In further embodiments, said hydrophilic cross-linker comprises polyethylene glycol. In further embodiments, more than half of said hydrophobic solvent by volume comprises molecules selected from the group consisting of water, ethylene glycol, and polyethylene glycol. In further embodiments, said hydrophilic cross-linker comprises a polyhedral silsesquioxane. In further embodiments, said hydrophilic cross-linker comprises octakis(hydridodimethylsiloxy)octasesquioxane.

In further embodiments, the invention relates to a method of making a polymer composite comprising: i) providing a cross-linker comprising polyethylene glycol disubstituted with acrylic groups; ii) mixing said cross-linker calcium phosphate aggregates, 2-hydroxyethyl methacrylate, and ethylene glycol under conditions such that a polymer composite is formed; and iii) mixing said composite with a solution comprising a peptide under conditions such that said polymer composite absorbs said peptide.

In further embodiments, the invention relates to a method of making a polymer composite comprising: a) providing: i) a cross-linker comprising polyethylene glycol disubstituted with acrylic groups, and ii) a biomolecule; b) mixing said cross-linker, biomolecule, calcium phosphate aggregates, 2-hydroxyethyl methacrylate, and ethylene glycol under conditions such that a polymer composite comprising said biomolecule is formed.

In some embodiments, an elastic composite comprises a polymer with a plurality of hydroxyl groups, preferably poly(2-hydroxyethyl methacrylate) (pHEMA), and calcium phosphate aggregates, preferably hydroxyapatite (HA). In some embodiments, composites are formed by crosslinking a polymer with a plurality of hydroxyl groups in the presence of different types of aggregates using aqueous ethylene glycol as a solvent. In further embodiments, composites are freeze-dried in order to remove residual water or other solvents. In further embodiments, composites have mineral-to-organic matrix ratios approximating those of dehydrated human bone. In further embodiments, composites exhibit fracture resistance.

In some embodiments, the invention relates to a material comprising: a) a polymer comprising a plurality of monomer subunits comprising hydroxyl groups; and b) aggregates; wherein said material is elastic. In further embodiments, said material is elastic after compressed with a force of between 0.5 and 1 MPa. In further embodiments, said material does not fracture under a compression of force between 29 and 100 MPa. In further embodiments, said monomer subunits are substituted or unsubstituted hydroxyalkyl acrylate subunits. In further embodiments, said monomer subunits are 2-hydroxyethyl methacrylate subunits. In further embodiments, said aggregates comprise a hydroxyl. In further embodiments, said aggregates comprise calcium salts. In further embodiments, said aggregates comprise calcium hydroxyapatite. In further embodiments, said aggregates comprise beta-tricalcium phosphate. In further embodiments, said aggregates comprise calcium hydroxyapatite of a size between 50 nanometers and 50 micrometers. In further embodiments, said aggregates are between 30%-70% by weight of the bulk material. In further embodiments, said polymer further comprises ethylene glycol subunits. In further embodiments, said material further comprises a component selected from the group consisting of ethylene glycol, polyethylene glycol, and water. In further embodiments, said bulk material contains less than 0.5% of water, ethylene glycol, and polyethylene glycol by weight. In further embodiments, said material further comprises cells, biomolecules, peptides, saccharides, polysaccharides, or portions thereof. In further embodiments, said material is biodegradable.

In some embodiments, the invention relates to a bulk material comprising: a) a polymer comprising substituted or unsubstituted hydroxyalkyl acrylate subunits and b) calcium phosphate aggregates; wherein said material is between 10%-90% by weight of said calcium phosphate aggregates. In further embodiments, said hydroxyalkyl acrylate subunits are 2-hydroxyethyl methacrylate subunits. In further embodiments, said calcium phosphate aggregates are calcium hydroxyapatite aggregates. In further embodiments, said calcium phosphate aggregates are beta-tricalcium phosphate aggregates.

In some embodiments, the invention relates to an elastic material thicker than 1 millimeter comprising: a) a co-polymer comprising 2-hydroxyethyl methacrylate and ethylene glycol subunits; and b) calcium hydroxyapatite; wherein said material is between 30%-70% by weight of said calcium hydroxyapatite.

In some embodiments, the invention relates to a method of making a polymer composite comprising: i) providing: a) an aggregate comprising a hydroxyl, b) a first monomer comprising a vinyl group and a hydroxyl, c) a second monomer comprising two vinyl groups and a hydrophilic linking group, and d) a hydrophilic solvent; and ii) mixing said aggregate, first monomer, second monomer, and solvent to form a solution under conditions such that a polymer composite is formed. In further embodiments, said solution further comprises a radical initiator. In further embodiments, said radical initiator is hydrophilic. In further embodiments, said radical initiator is selected form the group consisting of ammonium persulfate and sodium metasulfite. In further embodiments, said aggregates comprise calcium. In further embodiments, said aggregates comprise beta-tricalcium phosphate. In further embodiments, said aggregates comprise calcium hydroxyapatite. In further embodiments, said aggregates comprise calcium hydroxyapatite of a size between 50 nanometers and 50 micrometers. In further embodiments, said first monomer is a substituted or unsubstituted hydroxyalkyl acrylate. In further embodiments, said first monomer is 2-hydroxyethyl methacrylate. In further embodiments, said second monomer is selected from the group consisting of ethylene glycol disubstituted with moieties having a vinyl group and polyethylene glycol disubstituted with moieties having a vinyl group. In further embodiments, said second monomer is ethylene glycol dimethacrylate. In further embodiments, said hydrophilic solvent comprises ethylene glycol or polyethylene glycol. In further embodiments, said ethylene glycol or polyethylene glycol is between a 10%-70% to 100% volume ratio compared to the first monomer. In further embodiments, said hydrophilic solvent comprises water. In further embodiments, said water is between a 1%-40% to 100% volume ratio compared to the first monomer.

In some embodiments, the invention relates to a method of making a polymer composite comprising: i) providing: a) calcium phosphate aggregates; b) a substituted or unsubstituted hydroxyalkyl acrylate, c) a second monomer of ethylene glycol disubstituted with moieties having a vinyl group, d) liquid ethylene glycol, and e) a mold; and ii) mixing said salt aggregate, first monomer, second monomer, and liquid ethylene glycol to form a solution; and iii) forming a polymer composite in said mold. In further embodiments, said hydroxyalkyl acrylate is 2-hydroxyethyl methacrylate. In further embodiments, said calcium phosphate aggregates are calcium hydroxyapatite aggregates. In further embodiments, said calcium phosphate aggregates are beta-tricalcium phosphate aggregates.

The present invention contemplates utilizing the above-mentioned polymer embodiments as elastic osteoconductive composite bone grafts to augment the biochemical microenvironment of hard-to-heal bony defects resulting from aging, cancer, trauma or metabolic diseases, contributing to the more effective surgical treatment of these debilitating conditions.

### BRIEF DESCRIPTION OF THE FIGURES

FIG. 1 shows EDSs of the cross-sections of as-prepared FlexBone 37% commercial hydroxyapatite (HA) powder (37Com-3-AP) (top) and 37% commercial freeze-dried (FD) FlexBone 37Com-3-FD (bottom).

FIG. 2A shows compressive force-strain loading curves of FlexBone composites 37Com-3-AP (middle curve) and 37Com-3-FD (upper curve) versus that of the corresponding un-mineralized pHEMA (lower curve). The compressive stress corresponding to the highest strain (83.7%) reached is labeled next to each curve.

FIG. 2B shows 37Com-3-AP (top view) and 37Com-3-FD (top and side views) after being released from >80% compressive strains. Arrows indicate the small cracks formed along the edge of the freeze-dried composites upon compression.

FIG. 3 shows data of compressive behavior of FlexBone as a function of HA content, i.e., compressive loading and unloading force-strain curves of FlexBone samples 48Com-3-FD (solid line) and 41Com-3-FD (dotted line), respectively.

FIG. 4A shows data of structural integration and compressive behavior of FlexBone containing commercial polycrystalline HA vs. calcined HA, i.e., representative compressive loading and unloading force-strain curves of FlexBone 50Com-3-FD (solid curve) versus 50Cal-3-FD (dashed curve).

FIG. 4B shows data of structural integration and compressive behavior of FlexBone containing commercial polycrystalline HA vs. calcined HA, i.e., compressive stresses of FlexBone 50Com-3-FD (open bars) versus 50Cal-3-FD (crosshatched bars) at selected compressive strains (N=3).

FIG. 5 shows data of reversibility of the compressive behavior of as-prepared FlexBone. Repetitive loading and unloading force-strain curves of 40Cal-3-AP (solid curves) and 70Cal-4-AP (dotted curves) are at strains less than 40% (up to 1.4 MPa stress) 3 and 5 times, respectively.

FIG. 6A shows a XRD of a composite prior to cell seeding.

FIG. 6B shows a XRD of a composite (pre-seeded with 20,000-cells/cm² BMSC) 28 days after SC implantation in rat.

FIG. 7 shows data of size distribution of the calcined HA powders as determined by sedimentation measurements for particles with diameters below 10 μm. Both the SEM micrograph and the sedimentation measurement plot suggested a bimodal size distribution of the calcined HA powders with most of the particles sized 5 μm or below and the larger grains over 10 μm in size.

FIG. 8 illustrates the synthesis of macromer 2 wherein (i) is 15 eq. allyl alcohol, 6×10⁻⁴ eq. Pt(dvs), 20° C., 1 h, followed by 90° C., 1.5 h, N², 90%; (ii) is 40, 80 or 160 eq. rac-lactide, 200 ppm stannous octoate, 115° C., N², 20 h, >90%.

FIG. 9 shows data of in vitro degradation of urethane-crosslinked POSS-(PLAₙ)₈, or macromer 2 (FIG. 8), as a function of PLA polyester chain length (n=10, 20, 40) as a percentage of mass reduction of crosslinked macromer 2 in PBS buffer (pH 7.4) as a function of time. Squares: n=10; Circles: n=20; Triangles: n=40.

FIG. 10A illustrates a synthetic route for the attachment of CTA-1 to macromer 2 and the subsequent grafting of pHEMA to the macromer CTA by RAFT polymerization.

FIGS. 10B-10C illustrate a polymer matrix made using a diisocyanate cross-linker.

FIG. 10D illustrates certain embodiments of the invention having a polymeric matrix comprising a siloxane macromer crosslinked by a covalent linkage through a crosslinker comprising single or reactive groups. Panel (a): light shaded polymer=substituted siloxane: dark shaded polymer=polymeric segments containing: i) reactive groups (open teardrop) comprising hydroxyl, amino, carboxyly, (meth)acrylate, (meth)acrylamide, epoxy, alkyne, azido, alkoxysilane used for crosslinking; and ii) connecting groups (crosshatched teardrop) comprising hydroxyl, amino, carboxyly, (meth)acrylate, (meth)acrylamide, epoxy, alkyne, azido, alkoxysilane used for covalently attacing biomolecules. Panel (b) Crosslinkers comprising single or multi reactive groups comprising hydroxyl, carboxyly, (meth)acrylate, (meth)acrylamide, epoxy, alkyne, azido, alkoxysilane, et al. Specific examples include, but are not limited to, di-isocyanate, di-methaacrylate, or di-alkyne.

FIGS. 10E-10G illustrate certain embodiments of the invention as presented schematically in FIG. 10C.

FIG. 10E: The reactive group is a hydroxyl and the biomolecule attached to the connecting group is an integrin binding peptide.

FIG. 10F: The reactive group is an azido and the biomolecule attached to the connecting group is an HA-binding peptide.

FIG. 10G: The reactive group is a methacrylate and the biomolecule attached to the connecting group is an integrin-binding protein.

FIG. 11A shows RAFT data of GPC characterization of macromer 2 (dotted line: M_{w}/Mₙ (GPC)=1.23, n=20), macromer CTA (dashed line: M_{w}/Mₙ (GPC)=1.22, n=20).

FIG. 11B shows RAFT data of POSS-(PLAₙ-co-pHEMAₘ)₈ (M_{w}/Mₙ (GPC)=1.34; Mₙ(NMR)=222,000) n=20, m=200). Polydispersity (M_{w}/Mₙ) was determined using a PLGeI Mixed-D column on a Varian HPLC equipped with an evaporative light scattering detector. pHEMA=poly(2-hydroxyethyl)methacrylate; RAFT=radical addition fragmentation chain transfer polymerization.

FIGS. 12A-12C illustrate certain embodiments of the invention wherein crosslinking is performed by radical chemistry.

FIG. 13 illustrates certain embodiments of the invention where the mineral nucleating peptide is HA-binding peptide (SEQ ID No.: 1) and the cell adhesive ligand is (SEQ ID No.: 2).

FIG. 14A illustrates certain embodiments of the invention.

FIG. 14B illustrates certain embodiments of the invention, comprising (i) a random structure; (ii) a ladder structure; (iii) cage structure T₈, (iv) cage structure T₁₀; and (v) caged structure T₁₂.

FIG. 15 illustrates certain embodiments of the invention.

FIGS. 16A and 16B illustrate certain embodiments of the invention.

FIGS. 17A and 17B illustrate certain embodiments of the invention.

FIG. 18 illustrates the synthesis of (i) methacrylamides MA-C3-N3; and (ii) Gly-MA.

FIG. 19 illustrates the functionalization of an HA-binding peptide ((i) AK5-HA-12; and (iii) MA-C6-HA12) and an integrin binding peptide (GRGDS; (ii) AK5-GRDS; and (iv) MA-GRDS) with alkynyl and methacrylamido groups for subsequent covalent incorporation with the synthetic graft.

FIG. 20 illustrates the design of hybrid macromers containing a POSS nanoparticle core, a biodegradable PLA domain, an HA nucleation domain, a negatively charged growth factor retention domain and a cell adhesion domain. The block copolymer segments are sequentially grafted to POSS via ROP and RAFT polymerization.

FIGS. 21A and 21B illustrate the structures of macromer CTAs and synthetic routes for the preparation of star-shaped functional macromers. Arrows indicate the fragmentation sites of macromer CTA-1 and macromer CTA-2. The stable radicals generated upon fragmentation initiate the subsequent RAFT grafting of functional domains. Route 1 involves sequential RAFT grafting of the functional methacrylamides carrying polar peptide sidechains. Route 2 involves the RAFT grafting of azido-containing methacrylamide, followed by the conjugation of alkyne-terminating peptides to the macromer via the Cu(I)-catalyzed “click” chemistry.

Figure illustrates crosslinking macromers via the formation of urethane (A) and triazole (B) linkages. Cross-linkers PEG-diisocyanate and PEG-dialkyne are both synthesized from commercially available PEG. Crosslinking density in both cases can be varied, with the stoichiometric ratio of 1, 2 and 4 equivalents of cross-linker per polymer arm (or 8, 16 and 32 equivalents cross-linker per macromer) applied.

FIG. 23 illustrates polarized color light micrographs of H&E and ALP/TRAP stained FlexBone explants (50% HA, without exogenous growth factors) at four days (Panel A) and eight weeks (Panels B, C, & D). The penetration of bone marrow into the graft drill hole is evident by day four (Panel A), with extensive new bone formation within the drill hole (Panel B), at the FlexBone/marrow/cortical bone interface (Panel D), FlexBone/callus interface and FlexBone/cortical bone junction (Panel C) at eight weeks. New bone was stained red in H&E, with the resulting collagen fiber orientation shown in the polarized light micrographs. FlexBone remodeling is observed by eight weeks as indicated by extensive TRAP positive stains for osteoclasts (red arrows) at the surface of FlexBone followed by the ALP positive stains for osteoblastic activities (blue arrows). ALP=alkaline phosphatase; TRAP=tartrate-resistant alkaline phosphatase; H&E=hematoxylin and eosin; HA=hydroxyapatite.

FIG. 24 illustrates polarized color light micrographs of H&E and ALP/TRAP stained FlexBone explants (25% HA-25% TCP, pre-absorbed with 400 ng rhBMP-2/7) showing active remodeling of FlexBone by osteoclasts (red TRAP stains) as well as new bone formation (blue ALP stain) at the periphery of the FlexBone material. FB=FlexBone; NB=new bone; CB=cortical bone; C=callus; BM=bone marrow; ALP=alkaline phosphatase; TRAP=tartrate-resistant alkaline phosphatase; H&E=hematoxylin and eosin; HA=hydroxyapatite; rhBMP=recombinant human bone morphogenetic protein.

FIG. 25A illustrates an X-ray radiograph and micro-CT analysis of a 12-week explant of FlexBone (25% HA-25% TCP, pre-absorbed with 400 ng rhBMP-2/7) showing the callus completely bridging over the defect area and extensive new bone formation surrounding the entire FlexBone graft. RhBMP=recombinant human bone morphogenetic protein; micro-CT=micro-computed tomography.

FIG. 25B illustrates gray scale value histogram of the radiograph in FIG. 25A.

FIG. 25C depicts an isosurface view of the Flexbone graft in FIG. 25A.

FIG. 25D depicts an alpha blend view of the FlexBone graft in FIG. 25A.

FIG. 26A shows SEM micrograph of ComHA powders showing porous aggregates of polycrystalline HA.

FIG. 26B shows higher resolution SEM image of the circled area in FIG. 26A showing HA crystallites approximately 100 nm in size.

FIG. 26C shows grinded CalHA powders.

FIG. 26D shows particle size distribution of the CalHA as determined by sedimentation measurements for particles with diameters below 10 μm. Both SEM micrograph and the sedimentation measurement plot suggested a bimodal size distribution of CalHA powders with most particles sized 5 μm or below and the larger grains over 10 μm in size.

FIG. 27A: Compressive behavior of as-prepared FlexBone and pHEMA control as a function of mineral microstructure and content. Ten consecutive load-controlled loading-unloading cycles (3.0 N/min, 0.01 N to 18.0 N to 0.01 N) were applied to each specimen in ambient air using a Q800 DMA equipped with a compression fixture. a=ComHA-1-50; b=ComHA-1-37; c=CalHA-1-50; d=CalHA-1-37; and e=PHEMA.

FIGS. 27B and 27C: EDS of the cross-sections of FlexBone showing the removal of residue S-containing radical initiators upon equilibrating the as-prepared sample with water.

FIG. 27D: Compressive behavior of fully hydrated FlexBone and pHEMA control at body temperature as a function of mineral microstructure and content. Ten consecutive load-controlled loading-unloading cycles (3.0 N/min, 0.01 N to 10.0 N to 0.01 N) were applied to each specimen in water using a Q800 DMA equipped with a submersion compression fixture. a=ComHA-1-50; b=ComHA-1-37; c=CalHA-1-50; d=CalHA-1-37; and e=PHEMA. The hydrated FlexBone containing CalHA started to fail approaching >30% compressive strain during the first force ramping (denoted by *), thus did not continue with additional loading cycles.

FIG. 28A: Stress-strain curves showing freeze-dried FlexBone containing 50% ComHA is stiffer than the one containing 50% CalHA (solid line: ComHA-1-50; dashed line: CalHA-1-50). Unconfined displacement-controlled (approximately 0.015 mm/s) compression test was performed on a high capacity MTS with a 100-kN load cell.

FIGS. 28B and 28C: SEM of the cross-section of freeze-dried CalHA-1-50 before and after being compressed.

FIGS. 28D and 28E: SEM of the cross-section of freeze-dried ComHA-1-50 before and after being compressed.

The arrows in FIGS. 28C and 28E indicate the direction of compression.

FIG. 29A: SEM micrograph of a composite (pre-seeded with 20,000-cells/cm² BMSC) retrieved 28 days after SC implantation in rat;

FIG. 29B: SEM micrograph of a composite (without pre-seeded BMSC) retrieved 14 days after SC implantation in rat;

FIG. 29C XRD of the explanted sample shown in FIG. 29A, with diffraction patterns matching with that of the commercial HA powder;

FIG. 29D: ALP staining (dark area) of a 12-μm frozen section of an explanted composite (pre-seeded with 5×10³ cells/cm² BMSC) on day 14. Magnification: 400×.

FIG. 30A presents exemplary data of unconfined compression tests using as-prepared (37.0° C.) FlexBone composites as indicated by the slopes of stress-strain curves. 50% HA FlexBone composite: Green curve=0% TCH. 25% HA-25% TCP FlexBone composite: Dark Blue curve=0% TCH; Dark Purple curve=0.1% TCH; Yellow curve=0.5% TCH; Light Blue curve=2.0% TCH; and Light Purple curve=5.0% TCH.

FIG. 30B presents exemplary data of unconfined compression tests using hydrated (37.0° C.) FlexBone composites as indicated by the slopes of stress-strain curves. 50% HA FlexBone composite: Green curve=0% TCH. 25% HA-25% TCP FlexBone composite: Dark Blue curve=0% TCH; Dark Purple curve=0.1% TCH; Yellow curve=0.5% TCH; Light Blue curve=2.0% TCH; and Light Purple curve=5.0% TCH.

FIG. 30C presents one embodiment wherein a pice of fully hydrated FlexBone containing 25 wt % HA-25 wt % TCP is press-fitted into an 5-mm segmental defect in rat femur.

FIG. 31A presents exemplary data showing mineral component distribution in an elastic pHEMA matrix after incorporation of 0.2% TCH.

FIG. 31B presents exemplary data showing mineral component distribution in an elastic pHEMA matrix after incorporation of 0.5% TCH.

FIG. 31C presents exemplary data showing mineral component distribution in an elastic pHEMA matrix after incorporation of 2.0% TCH.

FIG. 31D presents exemplary data showing mineral component distribution in an elastic pHEMA matrix after incorporation of 5.0% TCH.

FIG. 31E presents exemplary data showing the microstructure of an as-prepared 0.5% TCH composite before repetitive (at least 10 cycles) of 1-MPa compression.

FIG. 31F presents exemplary data showing the microstructure of an as-prepared 0.5% TCH composite after repetitive (at least 10 cycles) of 1-MPa compression.

FIG. 31G presents exemplary data showing the microstructure of an as-prepared 2.0% TCH composite before repetitive (at least 10 cycles) of 1-MPa compression.

FIG. 31H presents exemplary data showing the microstructure of an as-prepared 2.0% TCH composite after repetitive (at least 10 cycles) of 1-MPa compression.

FIG. 32A presents exemplary data showing the in vitro release of various TCH incorporation loads from either FlexBone composites (dotted lines) or pHEMA hydrogels (solid lines). Blue=0.5% TCH; Green=1.0% TCH; Purple=2.0% TCH; and Red=5.0% TCH.

FIG. 32B presents exemplary data showing antibiotic activity of FlexBone-released TCH as indicated by sustained clear zone diameter between eight (8) and fifty (50) hours. Inset: Representative E. coli agar plate.

FIG. 33A presents exemplary data showing osteogenic trans-differentiation induction of a C2C12 culture without a graft carrier by rhBMP-2/7 (40 ng/ml) showing ALP activity across the culture plate.

FIG. 33B presents exemplary data showing osteogenic trans-differentiation induction of a C2C12 culture with a FlexBone graft by rhBMP=2/7 (40 ng/ml) showing localized ALP activity. (darkened area).

FIG. 34A presents exemplary data showing RAW264.7 osteoclast differentiation in the presence of a FlexBone graft pre-absorbed with 10-ng rmRANKL.

FIG. 34B presents exemplary data showing a lack of RAW264.7 osteoclast differentiation in the presence of un-mineralized pHEMA hydrogel pre-absorbed with 10-ng rmRANKL.

FIG. 34C presents exemplary data showing formation of TRAP-positive multinucleated osteoclasts in RAW264.7 culture supplemented with 10-ng rmRANKL every other day for six days.

FIG. 34D presents exemplary data showing a single 10-ng rmRANKL supplement was not sufficient to induce osteoclast differentiation in RAW264.7 culture.

### DETAILED DESCRIPTION OF THE INVENTION

The invention relates to composite materials that contain a polymer matrix and aggregates, and in some embodiments, methods of making and methods of using these materials. Preferably, the aggregates are calcium phosphate aggregates. Preferably, the materials are resistant to fracture. In further embodiments, the materials are used in surgical procedures of bone and joint replacement.

Hormonal therapies, small molecule inhibitors targeting key regulatory factors, and gene therapies that are commonly used for the treatment of musculoskeletal conditions typically do not provide instant relief of the symptoms of acute injuries and critical size defects. From this perspective, surgical reconstruction using proper bone grafts serves an important solution to traumatic defects induced by trauma, cancer, metabolic diseases and aging.

There are three types of bone grafts, autogenic, allogenic and synthetic. Disadvantages associated with autogenic grafting procedures include donor site morbidity, the frequent need for a second operation and an inadequate volume of transplant material. Allogenic bone grafts suffer from significant failure rates, mechanical instability, and immunological rejections. Synthetic grafts may be used in the reconstructive repair of skeletal defects. Preferred embodiments of the invention relate to grafts that are engineered to possess appropriate mechanical properties and integrated with bony tissue with good long-term viability.

Many synthetic scaffolds lack the ability to meet the combined structural, mechanical and biological requirements of a viable bone graft. Commercial synthetic bone grafts and substitutes may be made of ceramics, non-bioactive polymers or a combination of these components. Osteoconductive bioceramics include of poly(methyl methacrylate) (PMMA)-based bone cement, and polylactic acid (PLA), polyglycolic acid (PGA) and their copolymers. The bioceramics generally suffer from low fracture toughness. The average lifetime for PMMA bone cements that are used for bonding metal implants to bone in total joint replacement devices is ˜5 years, primarily due to their limited capacity to integrate with the bony tissue. Finally, the idea of locally delivering exogenous growth factors and cytokines by the grafts to compensate for the reduced healing potential at the defect site to induce proper host cell responses are often hampered by the lack of proper carriers capable of retaining and releasing these biomolecules in a confined environment. The PLA/PGA scaffolds, for instance, are poor binders for bone minerals and inefficient carriers for osteogenic growth factors.

Synthetic organic matrices can be designed to promote new bone formation. For instance, hydrogel scaffolds that degrade in response to matrix metalloprotease activity permit cell and bony tissue ingrowth, and self-assembling peptide amphiphiles have been engineered to template the nucleation of hydroxyapatite in vitro as disclosed in Hartgerink et al., Science, 1684-1688 (2001), incorporated herein by reference. A common limitation of these bioactive polymer scaffolds, however, is that they are mechanically weak, thus they are limited to treating small non/low-weight bearing craniofacial defects.

In one embodiment, the present invention contemplates a synthetic polymer and polymer-mineral composite grafts that provide structural support and mechanical stabilization to the site of fragile skeletal defects and simultaneously serve as a vehicle to locally deliver exogenous growth factors and cytokines to trigger proper host cell responses, promoting graft healing. In some embodiments, the disclosed composites are denoted as #Com/Cal-N-AP/FD, where # denotes the weight percentage of HA, Com for commercial HA, Cal for calcined HA, N for the type of hydrogel formulations (1, 2, 3 or 4), AP for as-prepared, and FD for freeze-dried. For instance, 70Cal-4-AP represents as-prepared FlexBone with 70% calcined HA that is formed using hydrogel formulation 4, whereas 40Com-3-FD represents freeze-dried FlexBone with 40% commercial polycrystalline HA that is formed using hydrogel formulation 3. Other objectives include: combining exogenous signaling molecules in order to introduce to the microenvironment of a defect to promote graft healing characterized by the remodeling, osteointegration and vascular ingrowth of the grafts; retaining and releasing bioactive signaling molecules to and from a synthetic graft in a sustained manner; integrating multiple desirable features including the ability to retain bioactive signaling molecules, biodegradability and cell adhesive properties into polymeric graft designs; and integrating osteoconductive bone mineral with the polymer scaffold with structural integration and mechanical properties to emulate the composite scaffold of bone.

It is not intended that embodiments of the invention be limited to any particular mechanism; however, it is believed that autogenic and allogenic bone graft healing is initiated by an inflammatory response, followed by vascular invasion and recruitment of mesenchymal stem cells (MSCs), a process similar to fracture healing. Although the later phase of graft repair and remodeling varies between dense cortical bone grafts and porous cancellous bone grafts, osteoclasts and osteoblasts are involved. The imbalance between resorption and bone formation can lead to graft failure. Further, new vessels are involved in osteogenesis and bone remodeling. They serve as a source of osteoblast and osteoclast precursors and signals for their recruitment. Vascular endothelial growth factor (VEGF) and receptor activator of nuclear factor κB ligand (RANKL), which regulate angiogenesis and osteoclastic bone resorption during skeletal repair, are down-regulated during allograft healing; this is believed to account for the high allograft failure rates. It is believed that RANKL and VEGF signals are sufficient to revitalize processed cortical bone to sustain long-term viability of clinical allografts. The introduction of the exogenous supply of these factors is believed to lead to bone resorption, neovascularization and revitalization of the necrotic bone.

In addition, bone morphogenetic proteins (BMPs), members of the transforming growth factor-β (TGF-β) superfamily, promote osteogenesis and fracture repair by inducing the differentiation of MSCs into bone-forming and cartilage-forming cells. Recombinant human bone morphogenetic protein-2 (rhBMP-2 or BMP-2/7 heterodiamer) has been approved by the Food and Drug Administration for clinical use as an adjuvant for spinal fusion and fracture union. Like osteoclast bone resorption, it is believed that osteogenesis is also dependent on sufficient vascularization. During the graft healing, endochondral ossification begins with the proliferation and aggregation of non-differentiated MSCs, which migrate along with new blood vessels and differentiate into osteoprogenitor cells and eventually give rise to bone formation. VEGF plays a role during this process.

In some embodiments, the invention relates to incorporating an exogenous supply of BMP-2, BMP-2/7 heterodimer, RANKL, and VEGF to a synthetic bone graft in order to induce host cell responses and elicit the coordinated remodeling and osteointegration of the grafts with vascular ingrowth. This combination of signals may either be introduced as recombinant proteins or delivered by gene therapy approaches. In further embodiments, it is contemplated that these growth factors and cytokines may be immobilized directly on the synthetic grafts. When administered parenterally BMP-2, RANKL, and VEGF fail to be retained within a local delivery site. Thus, in preferred embodiments, a synthetic carrier effectively retains and locally releases these exogenous proteins in a sustained manner, preferably throughout the early stage (first 3-5 days) of fracture/graft healing when the condensation of mesenchymal stem cells and the initiation of callus formation occur.

Sulfated polysaccharides such as heparin have an affinity for a number of basic growth factors including BMPs and VEGF. Using favorable electrostatic interactions, some embodiments of the invention relate to using polymer grafts functionalized with ionic domains bearing net charges opposite to those of the growth factors as a delivery vehicle for signaling molecules. Preferably, anionic domains are integrate into the synthetic graft to retain the basic recombinant growth factors such as, but not limited to, rhBMP-2 (pI: 9.3), rhVEGF165 (pI: 8.5), and rmRANKL (pI: 9.1, E. coli expressed),

One may introduce multiple functional domains (e.g. cell adhesive and anionic ligands) to a hydrogel scaffold by copolymerizing functionalized methacrylate or methacrylamide monomers as disclosed in Song et al., J. Am. Chem. Soc. 127, 3366-3372 (2005), incorporated herein by reference. However, the amount of anionic ligands that can be incorporated without causing phase-separation is limited. For instance, the attempt of integrating high percentages of anionic monomers (>10-20%) in the hydrogel copolymer would leave a significant amount of anionic monomers unpolymerized, making the determination of the actual content and distribution of the anionic ligands within the hydrogel network difficult. This limitation, combined with the non-biodegradability of the carbon network, makes the conventional polymethacrylamides or polymethacrylates less desirable for the design of bioactive polymer bone grafts.

Thus, another object of embodiments of the invention relates to injectable and degradable organic-inorganic hybrid macromers sequentially grafted with bone mineral nucleation domains, anionic growth factor retention domains, and cell adhesion domains as the functional building blocks of a new class of bioactive bone grafts. Strengthened by silicon-based nanoparticles, these hybrid macromers are modularly functionalized with the multiple functional domains using controlled ring-opening polymerization (ROP) and reverse addition fragmentation transfer (RAFT) polymerization in combination with efficient bioconjugation chemistries. Upon crosslinking these macromers under mild physiological conditions and retaining exogenous bioactive signaling molecules, synthetic bone grafts for stabilizing and repairing skeletal defects with healing capacities can be obtained.

The inorganic component of bone, calcium phosphate and the various calcium apatites support functions of the skeleton including calcium homeostasis, protection of soft organs and structure and locomotion with muscle tissue. The bending and compression strength of human bone correlates to bone mineral content. The quantity and quality of the deposited mineral (crystal size, maturity and structural integration with the organic matrices) influences the mechanical properties of bone. Proteins such as osteopontin and bone sialoprotein bind to HA crystals, and embodiments of the invention contemplate the use of calcium phosphates as carriers for the delivery of growth factors.

In some embodiments, the invention relates to integration of osteoconductive calcium apatite, particularly at high mineral content approximating that of human bone with the bioactive polymer bone grafts to enhance both the mechanical and biological performance of synthetic bone grafts. In other embodiments, using a urea-mediated HA-mineralization process, a surface layer of HA with varying morphology and crystallinity provides mineral-polymer interfacial adhesion.

In certain embodiments, the invention relates to HA-binding peptides and there use to template the nucleation and growth of aggregates preferably HA aggregates.

In further embodiments, the invention relates to covalently incorporating the HA-binding peptides to the mineral nucleation domain of the polymer graft to facilitate template-driven HA-mineralization in situ and prepare polymer-mineral composite grafts with substantial calcium apatite content.

In further embodiments, the invention relates to polymer siloxanes, preferably octakis(dimethylsiloxy) octasilsesquioxane (POSS), even more preferably octahedral hydroxylated POSS, and even more preferably octahedral hydroxylated POSS substituted with biodegradable polylactide (PLA) as disclosed in U.S. Provisional Patent Application No. 60/925,329, filed Apr. 19, 2007. As materials fabricated from polymer siloxanes, preferably substituted with polylactide have shape memory properties, it is contemplated that certain embodiments of the invention relate to a self-forming synthetic bone graft for fracture repair and cements that lead to better alignment and fixation between grafts and surrounding bony tissues upon heat activation.

In some embodiments, the invention relates to core structures of a macromer that act as building blocks for the addition of various functional domains. In preferred embodiments, the macromer is an initiator for RAFT polymerizations.

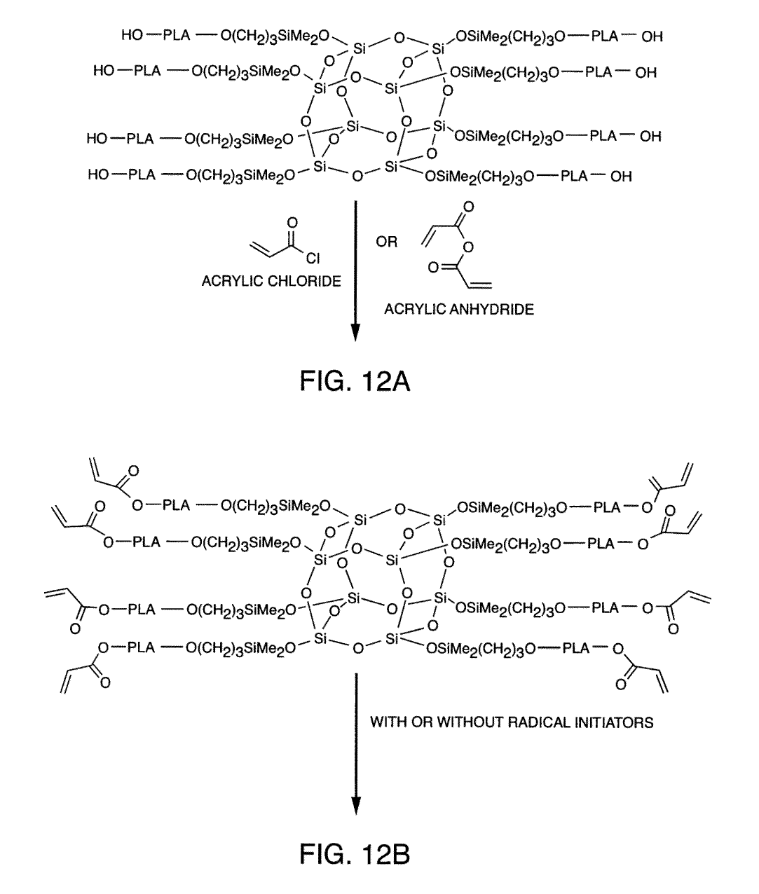

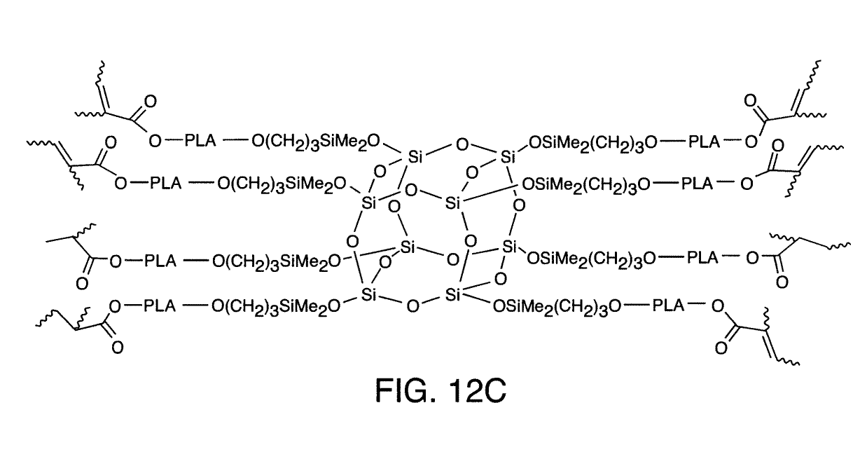

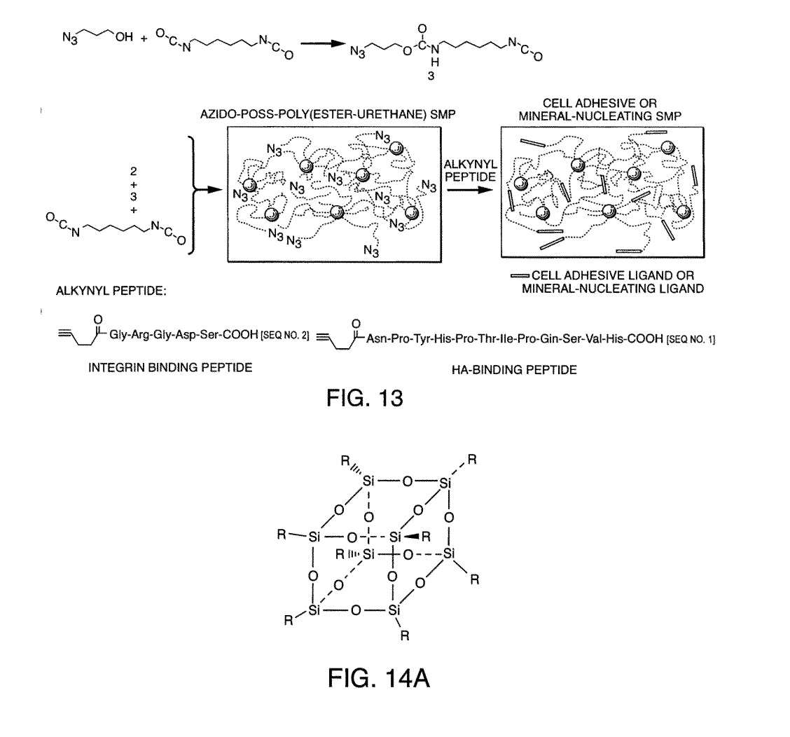

In additional embodiments, the invention relates to Si-based nanoparticles that are anchors for grafting polymer domains in bone grafts. One can crosslink any of the star-shaped macromers in the presence of varying percentages of HA and/or TCP powders using appropriate cross-linkers. One chooses a cross-linker depending on the functional groups substituted on the macromers. For instance, with the POSS-(PLAₙ)₈ macromer, since the terminus of each arm is a free hydroxyl, one uses a diisocyanate cross-linker (via urethane linkages). For those macromers with additional polymer blocks grafted to each PLA arm via RAFT polymerization, the cross-linker could depend on the functional groups, preferably the terminal functional group, displayed on the side chains on the grafted polymer blocks. In the case of POSS-(PLAₙ-co-pHEMAₘ)₈, one can crosslink with a diisocyanate since the pHEMA block contains hydroxyl side chains. Alternatively, one can terminate POSS-(PLAₙ)₈ or POSS-(PLAₙ-co-pHEMAₘ)₈, with alkylacrylates containing hydroxyl side chains as illustrated in FIG. 12. It is also contemplated that for macromers containing functional blocks displaying azido side chains, preferably terminal azido groups, one can use acetylene-based cross-linkers. FIG. 13 illustrates how one can incorporate HA-binding peptides to template the nucleation and growth of HA.

Although it is not intended that embodiments of the invention be limited to any particular mechanism, it is believed that the hydroxyl residues on pHEMA play a role in bonding with HA/TCP, thus giving rise to the impressive structural integration of the pHEMA matrix with the mineral component in FlexBone. Similar bonding likely occurs between the HA/TCP with the crosslinked POSS-(PLAₙ-co-pHEMAₘ)₈ matrix. It is not intended that for certain embodiments, the percentages of HA/TCP to be embedded in the crosslinked macromer matrices be limited to any particular aggregate or mineral incorporation. It is also contemplated that HA-binding peptides can be incorporated in order to template the nucleation and growth of HA.

Because of their hydrophilic nature, synthetic hydrogels such as poly(2-hydroxyethyl methacrylate), pHEMA, and functionalized derivatives are useful in a wide range of biomedical applications. With physical properties similar to natural gel-like extracellular matrices (ECM), these hydrogel polymers may be utilized in ophthalmic devices, soft tissue engineering scaffolds, carriers for drug or growth factor delivery, dental cements and medical sealants. For bone implant materials, it is desirable to fabricate composites containing pHEMA with high-weight percentages of hydroxyapatite (HA), an inorganic component of natural bone.

Song et al., JACS 125, 1236-1243 (2003), Song et al., J. Eur. Ceram. Soc. 23, 2905-2919 (2003), and Song et al., JACS 127, 3366-3372 (2005), corresponding to U.S. Patent Application Publication No. 2004/0161444, all of which are incorporated herein by reference, disclose a urea-mediated mineralization method integrating calcium phosphate, e.g., HA, on the surface of pHEMA hydrogels. Surface growth resulted in the formation of crystalline layers that may be detached from the hydrogel. However, aside from the surface, the interior of the urea-modified hydrogels contained small concentrations of calcium. A material with the flexibility and strength to integrate HA within the pHEMA-based hydrogels at a high mineral-to-gel ratio throughout the bulk scaffold was, until now, unachievable. The design of synthetic bone substitutes that mimic both the structural and mechanical properties of bone and exhibit desirable surgical handling characteristics is an objective of preferred embodiments of inventions disclosed herein.

### Polymer Composite Graphs

Poly(2-hydroxyethyl methacrylate) (pHEMA)-hydroxyapatite (HA) composites possessing osteoconductive mineral content approximating that of human bone and fabrication is disclosed. A preferred approach involves the formation of crosslinked pHEMA hydrogel in the presence of different types of HA powder using viscous aqueous ethylene glycol as a solvent. Despite the high HA content, these composites, termed “FlexBone”, are elastic and have unexpectedly high fracture resistance under physiological compressive loadings. Tailored microstructural property and compressive behavior of the composites can be achieved by the selective use of HA powder of varied sizes and aggregation and the composition of the organic component(s). When subcutaneously implanted in rats, it was observed that the HA component slowly dissolved and osteoblastic differentiation of the bone marrow stromal cells pre-seeded on the substrates. The unique fracture resistance to compressive loading and the elastomeric properties that ensure better accommodation to the inherent micro movement of bone at bone-graft interface make FlexBone a preferred composite for orthopedic applications.

The preparation of a class of elastomeric pHEMA-HA composite, FlexBone, comprising a high percentage (up to 70%) of osteoconductive HA is disclosed. These materials are able to withstand up to 700 megapascal compressive loads and over 70-80% strain without exhibiting brittle fracture despite having high mineral contents. The pre-polymer hydrogel cocktail formulation and the post-solidification processing conditions affect the compressive strength and elasticity of the FlexBone composites. The viscosity of ethylene glycol, the co-solvent used along with water during the fabrication of FlexBone composites, facilitated the dispersion of HA within the hydrogel scaffold, thereby preventing the HA particles from settling to the bottom of the mold during solidification. The high-boiling point of ethylene glycol also contributed to the long-lasting elasticity observed with the as-prepared FlexBone composite crosslinked in high-ethylene glycol-content media.

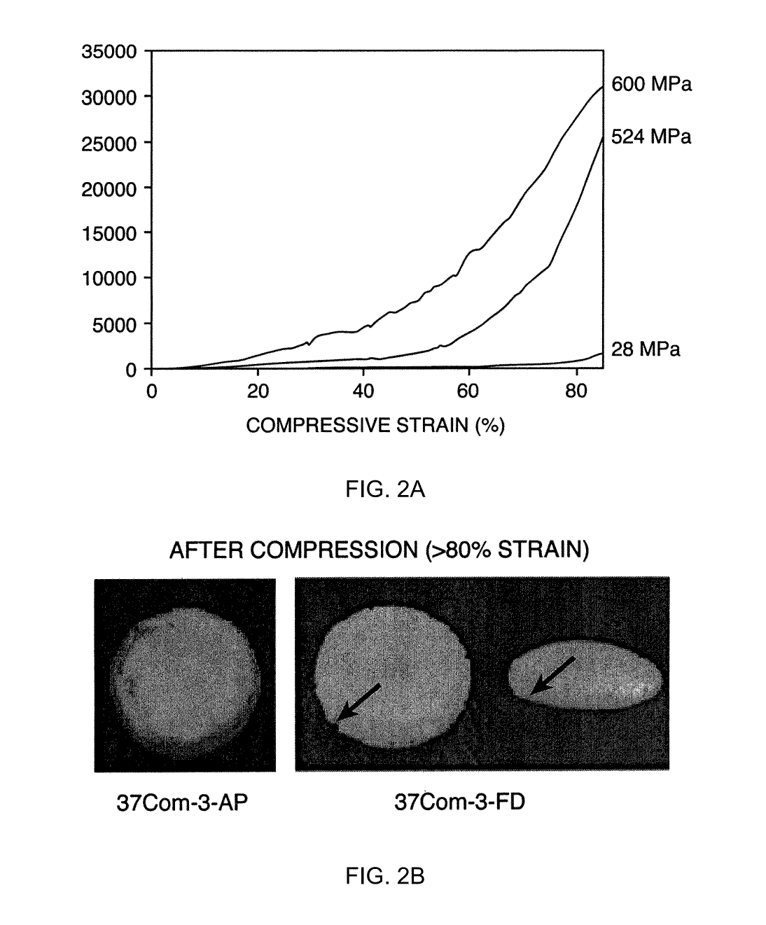

Reversible compressive behavior of as-prepared FlexBone under a few megapascal compressive loads and strains up to 40% suggest that these materials may be used in treating low to moderate weight-bearing skeletal defects with less dependence on additional surgical fixations (e.g. via rods or plates). Although the degree of crosslinking of the pHEMA matrix was kept constant at 2% for experiments thus far, it is contemplated that this value can be readily altered to either enhance the mechanical strength or improve the elasticity of the composite. The enhancement of stiffness and strength upon freeze-drying as exemplified in FIGS. 2A and 2B was observed with all FlexBone formulations investigated.

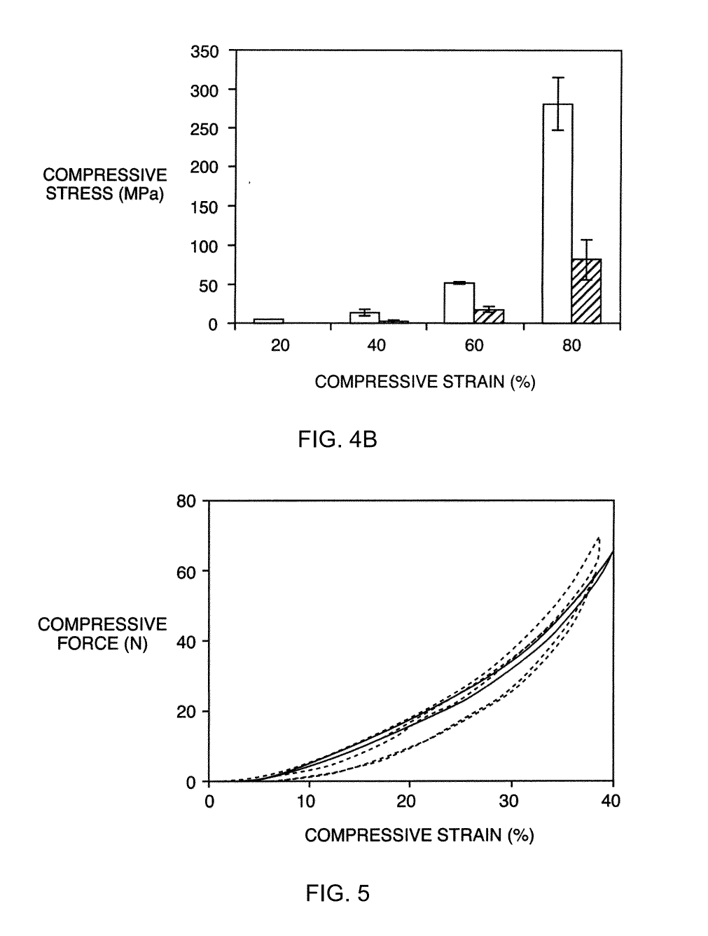

Our findings demonstrate that the compressive behavior and microscopic structural response to compression exhibited by the FlexBone composite was dependent on the size and aggregation of the HA particles incorporated. Whereas the more compact calcined HA particles were advantageous for the preparation of FlexBone with very high HA content (>50%), from a material processing point of view, the porous aggregates of HA nanoparticles in the commercial powder led to the formation of stronger composites. The submicrometer scale aggregation of HA nanoparticles in the commercial powder acted as “sponges”, absorbing the pre-polymer hydrogel cocktail and yielded larger surface contact areas between the hydrogel and the HA powder. This property contributed to better structural integration of the composite and to stronger and tougher compressive behavior in FlexBone containing commercial instead of calcined HA (FIGS. 4A and 4B).

SEM studies further elucidated that a contributing factor for the observed differences in compressive behavior is the ability for the spherical HA nanocrystal aggregates in the commercial HA-containing FlexBone composite to flatten into plywood-like structures upon compression. The combination of the soft hydrogel with the hard apatite crystals is unique.

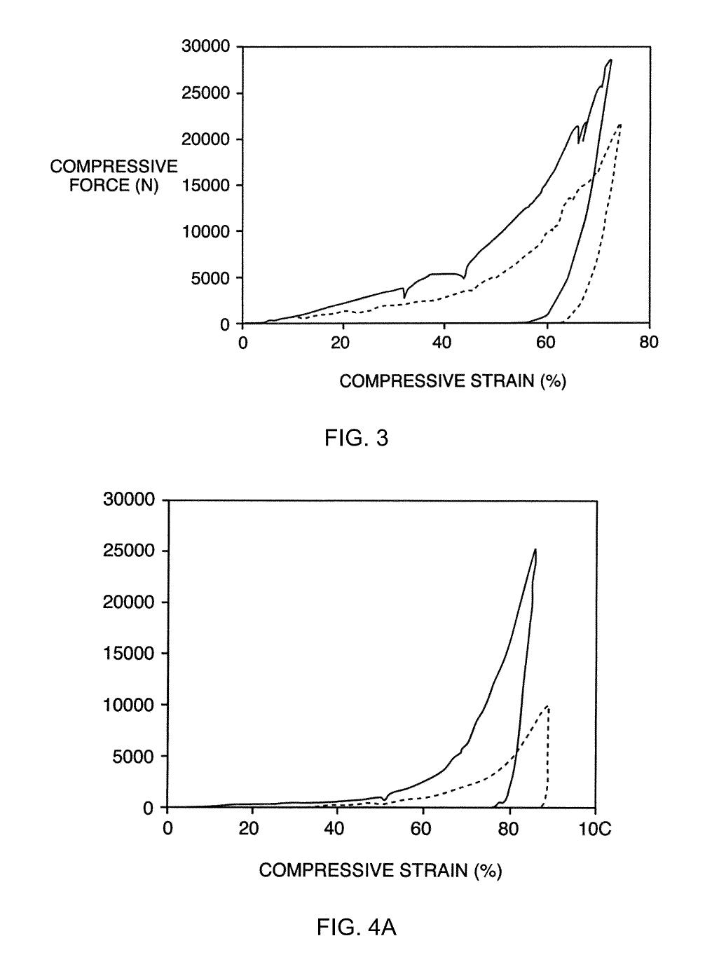

The compressive behavior of the FlexBone composite is dependent on its mineral content, a property that is useful in tailoring FlexBone for clinical applications ranging from craniofacial defects to weight-bearing fractures. The work under the force-strain curves of FlexBone samples increased with increasing mineral content, suggesting that FlexBone samples with higher percentages of HA are generally stiffer, tougher, and stronger. This trend, as representatively shown in FIG. 3, applied to FlexBone containing calcined HA powder as well and is in agreement with those observed with natural bone, where the tensile Young's modulus of compact bone shows a strong positive correlation with the mineral content. The force-strain curves obtained with freeze-dried mineralized samples are characteristically less smooth than those obtained with unmineralized pHEMA control gel or as-prepared composite gels. This may be due in part to the micropores generated by the removal of water during the freeze-drying process.

Subcutaneous implantation of FlexBone pre-seeded with bone marrow stromal cells (BMSC) in rats showed that the mineral component slowly dissolved over time and the pHEMA matrix, combined with the osteoconductive HA component, provided a cytocompatible environment to support the attachment, penetration and osteogenic differentiation of BMSC in vivo performed on thin substrates (1-mm in thickness). A preferred synthetic bone graft is designed to fill an area of defect to provide structural stabilization and to promote the healing and repair of the skeletal lesion. The synthetic grafts eventually remodel and become replaced by newly synthesized bone. From this perspective, biodegradability, osteoconductivity and osteoinductivity of the synthetic bone grafts are desirable along with mechanical strength and elastomeric properties that facilitate its surgical fitting to the defect site.

One object of embodiments of the invention is to provide biodegradability of the organic matrix of the composite grafts in order to enhance the in vivo dissolution rate of the osteoconductive mineral component (e.g. by using a more soluble β-tricalcium phosphate, β-TCP, to the HA mineral phase), and locally retaining and releasing osteoinductive growth factors and cytokines on and from the synthetic scaffold.

Embodiments of the invention contemplate lightweight pHEMA-HA composites containing between 40%-80% HA and even more preferably 50%-80% HA. These composites may be prepared using a variety of hydrogel formulations and HA particles. The adjustable parameters of the composite formulations allowed engineered FlexBone with a range of compressive strength and stiffness. FlexBone composites exhibit strong organic-inorganic material integration throughout the 3-D network, and did not undergo brittle fracture under high compressive stress despite their high mineral content. The elasticity of the as-prepared composites facilitate better fitting (by compression) of FlexBone into an area of bone defect.

In certain embodiments, the invention relates to polymerizable composite formulations injected into a defect site to allow for in situ solidification. Upon implantation, a synthetic graft possessing elastomeric properties may accommodate the inherent micro movement of bone, particularly at the bone-graft interface, thus reducing potential graft failure. The fracture resistant compressive behavior of FlexBone and its ability to slowly reabsorb and template the osteoblastic differentiation of BMSC in vivo makes FlexBone a preferred candidate for craniofacial applications and for treatment of bony defects requiring moderate load-bearing capability.

The strong organic/inorganic interface achieved with FlexBone demonstrates that non-covalent binding between apatite crystals and a highly hydroxylated hydrogel can be exploited in the rational design of new bonelike composites. In addition, the different mechanical and structural responses to compression exhibited by composites containing calcined HA versus loosely aggregated nanometer-sized HA suggest that the size and morphology of the inorganic component are significant parameters in the rational design of composites.

In further embodiments, the invention relates to antibiotics and bioactive signaling molecules related to osteoblast differentiation attached to composite graphs disclosed herein. The signaling molecules may be covalently attached to or non-covalently trapped within the hydrogel scaffold of the composite. A range of in vivo resorption rates may also be engineered via the use of HA in combination with other calcium phosphate particles, such as β-TCP, that have desired in vivo dissolution rates for remodeling.

In further embodiments, the invention relates to seeding or loading FlexBone with bone marrow stem cells prior to surgical implantation. A Flexbone graph loaded with cells can be applied to a removed femoral segmental as provided in Example 9. The loading of grafts with bone marrow stem cells prior to implantation enhances the ability of the graph to integrate with host tissue, vascularize, and heal.

Further embodiments of the invention relate to 1) pre-load growth factors and cytokines, gene vectors, or retroviruses on Flexbone prior to surgical implantation; 2) pre-load FlexBone with cells prior to implantation; or 3) pre-load growth factors and cytokine, gene vectors, retroviruses plus cells in FlexBone prior to implantation. All these approaches may optionally be combined with the pre-drilling holes in FlexBone. In preferred embodiments, the gene vector encodes BMP-2, BMP-2/7 heterodiamer, RANKL and VEGF. In more preferred embodiments, the gene vectors are recombinant adeno-associated viruses, rAA-BMP-2, rAA-BMP-2/7 heterodiamer, rAA-RANKL and rAA-VEGF prepared as disclosed or appropriately modified in Ito et al., Nature Medicine 11(3):291-297 (2005).

As used herein, a “polymer” refers to any covalent arrangement of atoms made up of repeatedly linked subunits. Within certain embodiments, it is preferred that the number of repeating moieties is three or more or greater than 10. The linked moieties may be identical in structure or may have variation of structure, i.e., co-polymer. In a preferred embodiment, the polymer is made up of moieties linked by ester groups, i.e., polyester. Polyesters include polymer architecture obtained through stereoselective polymerizations. Polylactone means a polyester of any cyclic diester, preferably the glycolide the diester of glycolic acid, lactide, the diester of 2-hydroxypropionic acid, ethylglycolide, hexylglycolide, and isobutylglycolide, which can be produced in chiral and racemic forms by, e.g., fermentation of corn. Metal alkoxide catalysts may be used for the ring-opening polymerization (ROP) of lactones. In the presence of chiral catalysts, each catalyst enantiomer preferentially polymerizes one lactone stereoisomer to give polymer chains with isotactic domains.

As used herein, a “peptide” refers to compounds containing two or more amino acids linked by the carboxyl group of one amino acid to the amino group of another. It is contemplated to include enzymes, receptors, proteins and recombinant proteins. It is contemplated that they may be purified and/or isolated from natural sources or prepared by recombinant or synthetic methods. The amino acids may be naturally or non-naturally occurring or substituted with substituents.

As used herein, a “composite” refers to two or more constituent compositions that remain distinct on a macroscopic level, preferably approaching nanometer dimensions, within a finished structure. In a preferred embodiment, the composite material has a polymer component and an aggregate component. It is not intended that embodiments of the invention be limited to any particular mechanism, but it is believed that the molecular properties of the polymer, particularly the hydrophobicity of monomer subunits provides desirable adherence of the aggregates to the polymer matrix. The “polymer matrix” refers to the surrounding polymer within which aggregates are contained. It is contemplated that such a matrix may be porous or non-porous.

As used herein, “hydroxyalkyl acrylate” refers to a compound having the general formula:

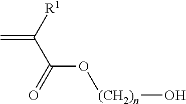

wherein R¹ is hydrogen or alkyl and n is 1 to 22. A preferred hydroxyalkyl acrylate is 2-hydroxyethyl methacrylate, where R¹ is methyl and n is 2, having the formula:

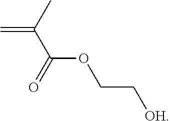

As used herein, “monomer subunits” of a polymer refers to the repeating structure that results from the polymerization process of monomers. In a preferred embodiment, subunits of 2-hydroxyethyl methacrylate have the following repeating representative structural formula:

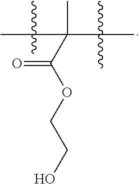 [SMILES: *C(*)(C)C(=O)OCCO]

As used herein, a “siloxane macromer” refers to a siloxane substituted with three or more crosslinking groups and/or polymer(s). The linking groups and/or polymers may be the same or different.

As used herein, a “cross-linker” refers to any variety of molecular arrangements that upon a chemical reaction covalently bonds one molecular entity, e.g., polymer, monomer, biomolecule, and/or macromer, to another. It is intended to include crosslinking between different molecular entities. Preferably, a cross-linker comprises a linking group terminally substituted with a reactive group, or two or more reactive groups. The two reactive groups may be different. Examples of preferred cross-linkers are polyethylene glycol diacrylate, polyethylene glycol diisocyanate, and hexamethylene diisocyanate.

As used herein, a “linking group” refers to any molecular arrangement for connecting chemical moieties. Examples include disubstituted groups such as, but not limited to, alkyl, substituted alkyl, polyethylene glycol, substituted polyethylene glycol, alkylamine, substituted alkylamine, polyalkylamine, substituted polyalkylamine, alkylthiol, substituted alkylthiol polyalkylthiol, substituted polyalkylthiol, alkylamide, substituted alkylamide, polyalkylamide, substituted polyalkylamide, alkylthioester, substituted alkylthioester, polyalkyl thioester, a substituted polyalkylthioester, alkylthioamide, substituted alkylthioamide, polyalkylthioamide, substituted alkylthioamide groups and combinations thereof.

As used herein, “hydroxyl” refers to an oxygen atom covalently bound to a hydrogen atom. It is contemplated that the oxygen atom may be further covalently or non-covalently bound to other atoms, including, but not limited to, carbon, metals, and metalloids. It is also contemplated that hydroxyl may be a hydroxyl ion.

The term “alkyl”, as used herein, means any straight chain or branched, non-cyclic or cyclic, unsaturated or saturated aliphatic hydrocarbon containing from 1 to 10 carbon atoms, while the term “short chain alkyl” has the same meaning as alkyl but contains from 1 to 4 carbon atoms. The term “long chain alkyl” has the same meaning as alkyl but contains from 5 to 22 carbon atoms. Representative saturated straight chain alkyls include, but are not limited to, methyl, ethyl, n-propyl, n-butyl, n-pentyl, n-hexyl, n-septyl, n-octyl, n-nonyl, and the like; while saturated branched alkyls include, but are not limited to, isopropyl, sec-butyl, isobutyl, tert-butyl, isopentyl, and the like. Cyclic alkyls may be obtained by joining two alkyl groups bound to the same atom or by joining two alkyl groups each bound to adjoining atoms. Representative saturated cyclic alkyls include, but are not limited to, cyclopropyl, cyclobutyl, cyclopentyl, cyclohexyl, and the like; while unsaturated cyclic alkyls include, but are not limited to, cyclopentenyl and cyclohexenyl, and the like. Cyclic alkyls are also referred to herein as a “homocycles” or “homocyclic rings.” Unsaturated alkyls contain at least one double or triple bond between adjacent carbon atoms (referred to as an “alkenyl” or “alkynyl”, respectively). Representative straight chain and branched alkenyls include, but are not limited to, ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include, but are not limited to, acetylenyl, propynyl, 1-butynyl, 2-butynyl, 1-pentynyl, 2-pentynyl, 3-methyl-1-butynyl, and the like.

As used herein, “aggregates” refers to a collection of atoms or molecules that form a collective mass. It is intended that the atoms can be a part of organic molecules, alloys, salts, metallic salts, and minerals. It is not intended that the aggregate be limited to having any specific shape. In preferred embodiments, aggregates have a preferred size, i.e., largest diameter, of between or 50 nanometers and 500 micrometers, or greater than 50 nanometers.

“Calcium phosphate aggregates” refers to aggregates containing calcium or calcium ions together with phosphate, polyphosphate, orthophosphates, metaphosphates, pyrophosphates, hydroxyl or combinations thereof. Examples include hydroxyapatite and tricalcium triphosphate of both alpha and beta crystalline forms.

As used herein, “salts” refer to an array of anionic and cationic atoms or molecules. It is not intended to be limited to those that contain metal atoms.

As used herein, “minerals” refers to arrays of atoms that contain metal or metalloids and a substantial amount of nonmetal atoms. These arrays may contain ionic, coordinate or covalently bound atoms or complexes. Preferred minerals contain calcium, more preferably calcium phosphate such as beta-tricalcium phosphate, and even more preferably calcium hydroxyapatite.

As used herein, “elastic” materials refer to materials returning to or capable of returning substantially to an initial form or state after a substantial deformation, preferably more than a 10% deformation by volume without a fracture, and even more preferably a 20% deformation by volume without a fracture. It is not intended to refer to brittle material that fractures upon deformation of volume despite the fact that the material may have a very low and small elastic range. In preferred embodiments, materials disclosed herein are elastic upon applying a compressive load of up to 1.4 MPa, more preferably of up to 2.6 MPa, and even more preferably up to 7.0 MPa and greater.

As used herein, a “fracture” refers to a break, rupture, or crack. In preferred embodiments, materials disclosed herein do not fracture at forces up to 28 MPa, more preferably they do not fracture between 28 and 524 MPa, and even more preferably they do not fracture between 150 and 500 MPa.

The term “substituted”, as used herein, means at least one hydrogen atom of a molecular arrangement is replaced with a substituent. In the case of an oxo substituent (“═O”), in the case of a hydrocarbon to form a keto (“C═O”), two hydrogen atoms are replaced. When substituted, one or more of the groups below are “substituents.” Substituents include, but are not limited to, halogen, hydroxy, oxo, cyano, nitro, amino, alkylamino, dialkylamino, alkyl, alkoxy, alkylthio, haloalkyl, aryl, arylalkyl, heteroaryl, heteroarylalkyl, heterocycle, and heterocyclealkyl, as well as, —NRₐR_{b}, —NRₐC(═O)R_{b}, —NRₐC(═O)NRₐNR_{b}, —NRₐC(═O)OR_{b}—NRₐSO₂R_{b}, —C(═O)Rₐ, C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. In addition, the above substituents may be further substituted with one or more of the above substituents, such that the substituent comprises a substituted alky, substituted aryl, substituted arylalkyl, substituted heterocycle, or substituted heterocyclealkyl. Rₐ and R_{b} in this context may be the same or different and, independently, hydrogen, alkyl, haloalkyl, substituted alkyl, aryl, substituted aryl, arylalkyl, substituted arylalkyl, heterocycle, substituted heterocycle, heterocyclealkyl or substituted heterocyclealkyl.

An unsubstituted compound refers to the chemical makeup of the compound without extra substituents. For example, unsubstituted proline is a proline amino acid even though the amino group of proline may be considered disubstituted with alkyl groups.

As used herein, “ethylene glycol” refers to a compound represented by the formula HO(CH₂CH₂O)ₙH, where n is 1. Polyethylene glycol refers to said formula where n is greater than 1, preferably providing a compound with an overall molecular weigh of less than 40,000. A polymer subunit of polyethylene glycol is —(CH₂CH₂O)ₙ— where n is greater than 1.

As used herein, a “bulk” material refers to a material that is consistently homogeneous within the interior of the material and at or near the surface of the material. It is not intended that the material necessary be homogeneous on or near the surface. The atoms at or near the surface may be oxidized because of exposure to the atmosphere. It is also contemplated that a bulk material may be chemically modified in order to facilitate contacting or connecting other materials or in order to grow other material layers; however, it is not contemplated that these surface modifications significantly alter the composition of the interior of the bulk material.

As used herein, a “homogeneous” material refers to the atomic and molecular constituents that make up the material having substantially the same distribution throughout the material considering a 1 millimeter unit cell or less, preferably a 100 micrometer unit cell or less.

As used herein, a “pore” refers to an opening through which fluid may pass. In preferred embodiments, a pore is created in composite materials disclosed herein using a drill or laser by channeling through the material creating holes of substantially similar dimensions.

As used herein, “cells” refer to the structural unit of an organism consisting of a nucleus and organelles surrounded by a semipermeable cell membrane. It is not intended to be limited to live or functioning cells. In preferred embodiments, the invention relates to materials that contain, incorporate, attach, or bind stem cells, hematopoieitic stem cells, endothelial cells, adipocytes, smooth muscle cells, reticular cells, osteoblasts, stromal fibroblasts, osteocytes and even more preferably, bone marrow stromal cells and mesenchymal stem cells.

As used herein, “bone marrow cells” refers to both bone marrow stems cells and the cells bone marrow stem cells differentiate into. Examples of bone marrow stem cells include hematopoietic stem cells and mesenchymal stem cells. Examples of other bone marrow cells include, white blood cells (leukocytes), red blood cells (erythrocytes), platelets (thrombocytes), osteoblasts, chondrocytes, and myocytes.

“Saccharide” means a sugar or substituted sugar exemplified by, but not limited to glucoside, glucoside tetraacetate, mannoside, mannoside tetraacetate, galactoside, galactoside tetraacetate, alloside, alloside tetraacetate, guloside, guloside tetraacetate, idoside, idoside tetraacetate, taloside, taloside tetraacetate, rhamnoside, rhamnoside triacetate, maltoside, maltoside heptaacetate, 2,3-desoxy-2,3-dehydromaltoside, 2,3-desoxy-2,3-dehydromaltoside pentaacetate, 2,3-desoxymaltoside, lactoside, lactoside tetraacetate, 2,3-desoxy-2,3-dehydrolactoside, 2,3-desoxy-2,3-dehydrolactoside pentaacetate, 2,3-desoxylactoside, glucouronate, N-acetylglucosamine, fructose, sorbose, ribose, galactose, glucose, mannose, 2-deoxygalactose, 2-deoxyglucose, maltulose, lactulose, palatinose, leucrose, turanose, lactose, maltose, mannitol, sorbitol, dulcitol, xylitol, erythitol, threitol, adonitol, arabitol, rhamnitol, talitol, 1-aminodulcitol, 1-aminosorbitol, isomaltitol, cellobiitol, lactitol, maltitol, volemitol, perseitol, glucoheptitiol, alpha,alpha-glucooctitiol including polysaccharides, carbohydrates and polyols (i.e., compounds having a large ratio of primary and secondary protected or unprotected hydroxyl groups where if unprotected have a ratio of hydrogen to carbon atoms near 2:1). In a preferred embodiment, the invention contemplates materials that contain, incorporate, attach, or bind saccharides, preferably the polysaccharide heparin and hyaluronic acid.

As used herein, a “biomolecule” refers to substances found or produced, engineered or naturally, in living organisms. It is not intended to be limited to actually obtaining the molecule from a living organism, i.e., the biomolecule may be made synthetically (in vitro). Examples include, but are not limited to, peptides, proteins, enzymes, receptors, substrates, lipids, antibodies, antigens, and nucleic acids.

As used herein, a “biodegradable” material refers to a material that breaks down all or a portion of the material into smaller components when interfaced with a living environment, preferably for the purpose of expelling non-naturally occurring components.

As used herein, a “cytokine” refers to a protein or glycoprotein that is used in an organism as signaling compounds. It is intended to include homologues and synthetic versions. Examples include the IL-2 subfamily, non-immunological such as erythropoietin (EPO) and thrombopoietin (THPO), the interferon (IFN) subfamily, the IL-10 subfamily, IL-1 and IL-18, CC chemokines (CCL)-1 to -28, and CXC chemokines.

As used herein a “gene vector” refers to any sequence of nucleic acid that codes for a particular protein. In a preferred embodiment, the gene vector is a plasmid or virus, such as a retrovirus, adenovirus, adeno-associated virus, herpesvirus, or lentivirus. These may be recombinant. With regard to recombinant adenovirus vectors, it is preferred that the vector is an “empty-Ad”, i.e., Ad genes are eliminated, since they provide a decreased antigenic load. Recombinant adenoviruses are typically delivered with helperviruses that replicate and express multiple Ad genes when present as described in Chamberlain et al., U.S. Pat. No. 6,451,596 (2002) hereby incorporated by reference. It is also contemplated that one may use cell lines expressing several Ad genes in trans, rather than being supplied from a helper-virus provided that the trans-complementing cell line adequately expresses the required Ad gene functions.

As used herein a “subject” refers to any animal, preferably a human patient, livestock, or domestic pet.

As used herein, a “vinyl” or “vinyl group” means an ethylenyl group unsubstituted or substituted or with an alkyl (i.e., —CR²═CH₂, wherein R² is hydrogen or alkyl). 2-hydroxyethyl methacrylate comprises the vinyl group, —C(CH₃)═CH₂.

As used herein, a “hydrophilic” group refers to any molecular arrangement that contains enough atoms that participate in hydrogen bonding to dissolve in water, i.e., water-soluble. Examples of hydrophilic groups include, but are not limited to, hydroxyl, carboxylate, ether, amine, amide, sulfate, sulfite, phosphate, polyphosphate groups, and corresponding acids and salts thereof. A preferred hydrophilic linking group is polyethylene glycol.

As used herein, a “reactive group” refers to a molecular arrangement that spontaneously forms covalent bonds when mixed with a compound that has a corresponding functional group. Examples are vinyl groups, which react with radicals. Other examples include nucleophiles and electrophiles, which react with each other. For example, in certain embodiments of the invention, it is contemplated that compounds with acrylic groups react with radicals. In certain embodiments it is also contemplated that compounds that contain acrylic groups (i.e., CH2=CH—C(═O)—) react by acting as an electrophile in a “Michael Reaction” with compounds containing amine groups or thiol groups. Alternatively, nucleophiles may take part in the substitution of electron withdrawing groups on a carbonyl. For example, carboxylic acids are often made electrophilic by creating succinyl esters and reacting these esters with aminoalkyls to form amides. Other common nucleophilic groups are thiolalkyls, hydroxylalkyls, primary and secondary amines, and carbon nucleophiles such as enols and alkyl metal complexes. Some alternative methods of joining moieties using reactive groups are disclosed in Lemieux & Bertozzi, Trends in Biotechnology 16 (12): 506-513 (1998). For example, in the Staudinger ligation, i.e., “click chemistry”, an azide comprising moiety and an alkynyl comprising moiety react to form triazoles. Other methods of conjugation reactive groups are provided in Hang & Bertozzi, Accounts of Chemical Research 34(9) 727-73 (2001) and Kiick et al., Proc. Natl. Acad. Sci., 99(1): 2007-2010 (2002).

As used herein, a “radical” refers to species with a single, unpaired electron. Radical species can be electrically neutral, but it is not intended that the term be limited to electrically neutral species, in which case they are referred to as free radicals. Pairs of electrically neutral radicals may be formed via homolytic bond breakage. Heating chlorine, Cl₂, forms chlorine radicals, Cl•. Similarly, peroxides form oxygen radicals and peresters fragment to acyl radicals, which may decompose to lose carbon dioxide to give carbon radicals. Azo compounds eject nitrogen to give a pair of carbon radicals. Many polymers may be made by the chain radical addition of substituted vinyl moieties with radicals.

As used herein, a “radical inhibitor” refers to any additive including but not limited to a compound or protein that is added to a chemical for inhibiting the self-induced, free-radical polymerization of said chemical.

### Osteogenesis

Bone formation is highly coordinated, beginning with the commitment of mesenchymal stem cells (MSCs) to an osteogenic fate and their subsequent differentiation and maturation into the major bone-forming cells, the osteoblasts. This sequential progression is regulated, among other influences, by a diverse repertoire of growth and adhesive factors acting in autocrine/paracrine manners at specific developmental stages. Of particular interest are the fibroblast growth factor (FGF) family and their receptors (FGFR), which interact with cell-surface heparin sulfate proteoglycans (HSPGs) to coordinate cell-fate decisions.

The progression of bone progenitor cells through to the osteoblast phenotype is tightly controlled by a diverse repertoire of fibroblast growth factors (FGF) and their receptors (FGFR). Sequential stages of osteogenic commitment and differentiation into preosteoblasts are responsible for cell growth, followed by their subsequent maturation into the major bone forming cells, osteoblasts. Osteoblasts will later become surrounded and separated from other osteoblasts by the matrix they produce, and terminally differentiate into osteocytes. At each stage, different FGF ligands are important in bone formation. In particular, FGFs-2, -9 and -18 have been shown to act at each of the stages of proliferation, differentiation and maturation, and FGF-2 protects cells against apoptosis.

Surgery is the preferred treatment for patients who have a neoplastic process affecting the mandible. If the lesion is benign but has compromised the integrity of the mandible, resection and reconstruction of the mandible is appropriate. If the lesion is malignant and has gained access to the cancellous bone, resection is also appropriate to obtain adequate surgical margins. Segmental composite mandibulectomy is a preferred treatment.

In certain embodiments, the invention relates to the use of composites disclosed herein that contain cells and biomolecules that promote osteogenesis as a transport disc to grow new bone. Transport disc osteogenesis is used to grow new bone across a defect where bone has been lost. Typically, a segment of bone is osteotomized adjacent to the defect and moved slowly and continuously across the defect by the use of a mechanical device. New bone fills in between the two bone segments. The piece of bone or material being moved or transported is referred to as the transport disc.

Large populations of osteogenic cells in an intact periosteum will be present in patients where a simple mandibulectomy has been done with little associated soft-tissue resection. For example, in a patient who undergoes mandibulectomy for an extensive ameloblastoma that is confined to the mandible, much of the periosteum will be preserved. In such a case there will be abundant local tissue, which would assist transport disc osteogenesis as the disc is moved through the bony defect. By contrast, there will be little to no periosteum in patients who have had complex composite mandibular resections where there has been extensive associated soft-tissue resection.

For example, in a patient who undergoes a mandibulectomy for a squamous-cell carcinoma (SCC) of the alveolus, invasion of the surrounding soft tissue is likely. In such a case, there will be substantial resection of the periosteum and, therefore, little adjacent soft tissue to assist in the formation of a bony construct as the distraction disc is moved along the defect in the so-called distraction tunnel. For a patient who has undergone a complex mandibular resection, the tissue adjacent to the distraction tunnel might be exclusively revascularized, transplanted tissue and there might be no osteogenic tissue. As a result, the only periosteum that would be present to help the formation and consolidation of the construct would be that associated with the transport disc.

### Siloxanes

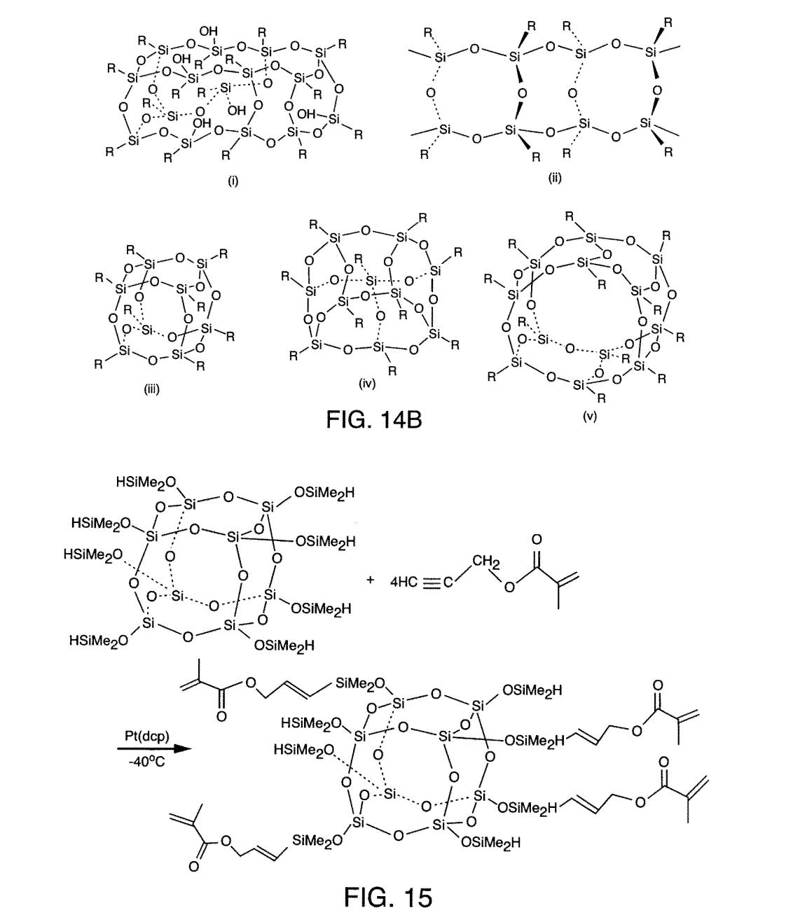

The preparation of siloxanes, including silsesquioxanes and metallasiloxanes, are described in Purkayastha & Baruah, Applied Organometallic Chemistry 2004, 18, 166-175. Silsesquioxane are compounds of an approximate formula of about RSiO_{1.5}, where R is any moiety but typically an alkyl, aryl, or substituted conjugate thereof. The compounds may assume a myriad of structures, including random, ladder, cage and partial cage structures (see FIG. 14B).

Silsesquioxanes are also sometimes termed ormosils (organically modified siloxanes). A preferred silsesquioxane is shown in FIG. 14A. To prepare monosubstituted silsesquioxane, there are several conventional synthetic routes. For example, the reaction of HSiCl₃ with PhSiCl₃ results in the formation of PhH₇Si₈O₁₂ via a co-hydrolysis reaction. A second route uses substitution reactions at a silicon center with the retention of the siloxane cage leading to structural modifications of silsesquioxane.

A variety of Polyhedral Oligomeric Silsesquioxanes (POSS) chemicals have been prepared which contain one or more covalently bonded reactive functionalities that are suitable for polymerization, grafting, surface bonding, or other transformations. Lichtenhan, J. D. et al., U.S. Pat. No. 5,942,638 (1999); Lichtenhan, J. D. et al., Chem. Innovat. 1: 3 (2001). Monomers have recently become commercially available as solids or oils from Hybrid Plastics Company (http://www.hybridplastics.com/), Fountain Valley, Calif. A selection of POSS chemicals now exist that contain various combinations of non-reactive substituents and/or reactive functionalities. Thus, POSS chemicals may be incorporated into common plastics via co-polymerization, grafting, or blending. Haddad et al., Polym. Prepr. 40: 496 (1999). Ellsworth, M. W. et al., Polym. News 24: 331 (1999).

Metallasiloxanes are siloxanes in which some of the silicon atoms have been replaced by a metal. Incorporation of metal into a siloxane framework can lead to two- and three-dimensional or linear networks. Metallasiloxanes may be derived from silanediols, disilanol, silanetriols and trisilanols. For example, the transesterification reaction of Ti(O-iPr)₄ with sterically hindered silanediol {(t-BuO—)₃SiO}₂Si(OH)₂ gives cyclic siloxane of the following formula:

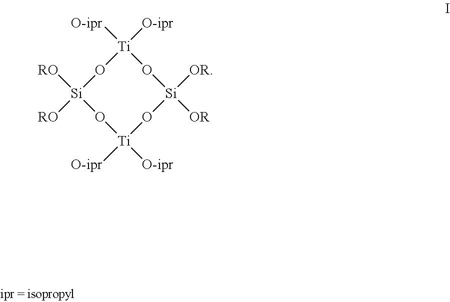

Similarly, cyclic dihalotitanasiloxanes [t-Bu₂Si(O)OTiX₂]₂ (X═Cl, Br, I) may be prepared by the direct reaction of titanium tetrachloride with t-Bu₂Si(OH)₂. Such compounds are made of eight-membered rings having the composition Ti₂Si₂O₄. Both silicon and titanium atoms in the molecule exhibit regular tetrahedral geometry. Analogously, the corresponding zirconium compound [t-Bu₂Si(O)OZrCl₂]₂ may be prepared from the reaction between the dilithium salt of t-Bu₂Si(OH)₂ and ZrCl₄.

Cyclopentadienyl-substituted titanasiloxane [t-Bu₂Si(O)OTiCpCl]₂ may be prepared directly by the reaction of CpTiCl₃ with t-Bu₂Si(OLi)₂. The reaction of the silanediol Ph₂Si(OH)₂ with the zirconium amido derivative Zr(NEt₂)₄ leads to the formation of the dianonic tris-chelate metallasiloxane [NEt₂H₂]₂[(Ph₄Si₂O₃)₃Zr]. In the case of zirconocene, six oxygen atoms in a distorted octahedral geometry coordinate the central zirconium atom.

Disilanols may also be used as building blocks for a variety of metallasiloxanes. The disilanols are capable of chelating to form six-membered rings containing the central metal. The reactions lead to Group 4 metallasiloxanes from disilanols. In a similar manner, metallasiloxane derivatives of Group 5, Group 7, Group 9 and main group metals may be prepared from disilanols. Reactions of silanediol and disilanols with titanium halides or titanium amides give cyclic titanasiloxanes. Three-dimensional titanasiloxanes can be prepared by the reaction of the titanium amide with silanol or silanediol. Such reactions serve as a synthetic pathway for preparation of model compounds for titanium-doped zeolites. Cubic titanasiloxanes can be prepared by a single-step synthesis from the reaction of titanium orthoesters and silanetriols. In an analogous manner, the three-dimensional networks of aluminosiloxane, indiumsiloxane, galliumsiloxane, etc. may be prepared from the reaction of trisilanols and MMe₃, where M=Al, In, Ga, etc. In many of these networks, cubic metallasiloxanes, M₄Si₄O₁₂ polyhedrons, are present.

### Synthesis of Polyhedral Oligomeric Silsesquioxanes

The preparation of oligomeric silsesquioxanes is generally described in Li et al., (2002) Journal of Inorganic and Organometallic Polymers 11, 123-154. Reactions leading to the formation of POSS may be characterized depending on the nature of the starting materials employed. One group includes the reactions giving rise to new Si—O—Si bonds with subsequent formation of the polyhedral cage framework. This class of reactions assembles polyhedral silsesquioxanes from monomers of the XSiY₃ type, where X is a chemically stable substituent (for example, CH₃, phenyl, or vinyl), and Y is a highly reactive substituent (for example, Cl, OH, or OR) as represented in Equation 1:

nXSiY₃+1.5nH₂O [symbol] (XSiO_{1.5})ₙ+3nHY (Equation 1).

Alternatively, POSS can form from linear, cyclic, or polycyclic siloxanes that are derived from the XSiY₃-type monomers.

The second class of reactions involves the manipulation of the substituents at the silicon atom without affecting the silicon-oxygen skeleton of the molecule. A number of substituents may be appended to the silicon oxygen cages [R(SiO_{1.5})]ₙ (n=8, 10, 12, and larger). Such substituents include alcohols and phenols, alkoxysilanes, chlorosilanes, epoxides, esters, fluoroalkyls, halides, isocyanates, methacrylates and acrylates, alkyl and cycloalkyl groups, nitriles, norbornenyls, olefins, phosphines, silanes, silanols, and styrenes. Many of the reactive functionalities are suitable for polymerization or co-polymerization of the specific POSS derivative with other monomers. In addition to substituents with reactive functional groups, non-reactive organic functionalities may be varied to influence the solubility and compatibility of POSS cages with polymers, biological systems, or surfaces.

### Multifunctional POSS Synthesis

POSS (RSiO_{1.5})ₙ, where R═H and n=8, 10, 12, 14, or 16, are structures generally formed by hydrolysis and condensation of trialkoxysilanes (HSi(OR)3) or trichlorosilanes (HSiCl₃). For example, (HSiO_{1.5})_{n,} where n=8, 10, 12, 14, or 16, is prepared by hydrolysis of HSiCl₃ involving the addition of a benzene solution of HSiCl₃ to a mixture of benzene and SO₃-enriched sulfuric acid. The hydrolysis of trimethoxysilane may be carried out in cyclohexane-acetic acid in the presence of concentrated hydrochloric acid and leads to the octamer. The hydrolytic polycondensation of trifunctional monomers of type XSiY₃ leads to cross-linked three-dimensional networks and cis-syndiotactic (ladder-type) polymers, (XSiO_{1.5})ₙ. With increasing amounts of solvent, however, the corresponding condensed polycyclosiloxanes, POSS, and their derivatives may be formed.

The reaction rate, the degree of oligomerization, and the yield of the polyhedral compounds formed under these conditions depend on several factors. For example, POSS cages where n=4 and 6 can be obtained in nonpolar or weakly polar solvents at 0 or 20° C. However, octa(phenylsilsesquioxane), Ph₈(SiO_{1.5})₈, is more readily formed in benzene, nitrobenzene, benzyl alcohol, pyridine, or ethylene glycol dimethyl ether at high temperatures (e.g., 100° C.).

Multifunctional POSS derivatives can be made by the condensation of ROESi(OEt)₃, as described above, where ROE is a reactive group. This reaction produces an octa-functional POSS, R′₈(SiO_{1.5})₈. Another approach involves functionalizing POSS cages that have already been formed. For example, this may be accomplished via Pt-catalyzed hydrosilylation of alkenes or alkynes with (HSiO_{1.5})₈ and (HMe₂SiOSiO_{1.5})₈ to form octakis(hydridodimethylsiloxy) octasesquioxane cages as shown in FIG. 15. Another example of the synthesis of multifunctional POSS derivatives is the hydrolytic condensation of modified aminosilanes. Fasce et al., Macromolecules 32: 4757 (1999).

### POSS Polymers and Copolymers

POSS units, which have been functionalized with various reactive organic groups, may be incorporated into an existing polymer system through grafting or co-polymerization. POSS homopolymers can also be synthesized. The incorporation of the POSS nanocluster cages into polymeric materials may result in improvements in polymer properties, including temperature and oxidation resistance, surface hardening and reductions in flammability.

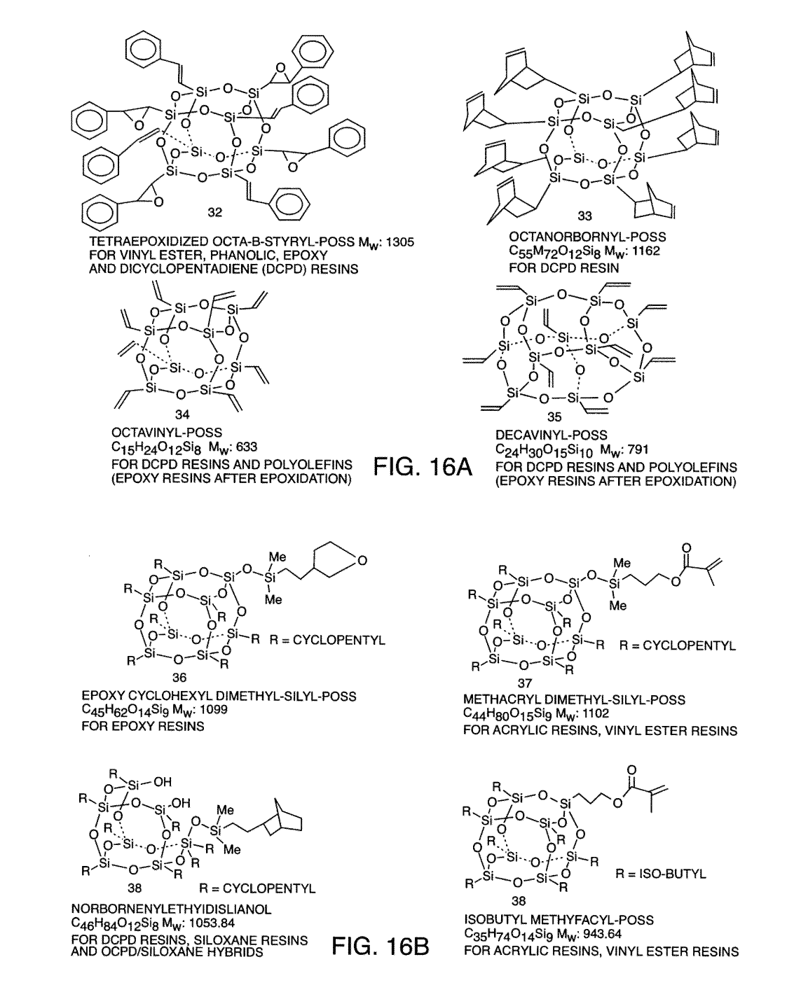

Different types of substituted POSS monomers may be chemically incorporated into resins. First, monofunctional monomers can be used. Alternatively, di- or polyfunctional POSS monomers can be used. Incorporating a monofunctional POSS monomer can actually lower the resulting resin's cross-link density if the amount of the monofunctional POSS monomers in the commercial resin employed is held constant. The POSS cages with organic functions attached to its corners have typical diameters of 1.2 to 1.5 nm. Therefore, each POSS monomer occupies a substantial volume. When that POSS monomer is monosubstituted, it cannot contribute to cross-linking. A 2 mol % loading of POSS in a resin might actually occupy 6 to 20 vol % of the resin, and this occupied volume contains no cross-links. Therefore, the average cross-link density will be lowered. Conversely, when a polyfunctional POSS monomer is employed, several bonds can be formed from the POSS cage into the matrix, thereby making the POSS cage the center of a local cross-linked network. Some examples of monofunctional and polyfunctional POSS monomers are illustrated in FIG. 16 together with the types of resins into which they may be chemically incorporated. Epoxy, vinyl ester, phenolic, and dicyclopentadiene (DCPD) resins may be made in which various POSS macromers are chemically incorporated. Besides the applications in nanoreinforced polymeric materials, there are other applications for POSS molecules as a core for building types of dendritic macromolecules.

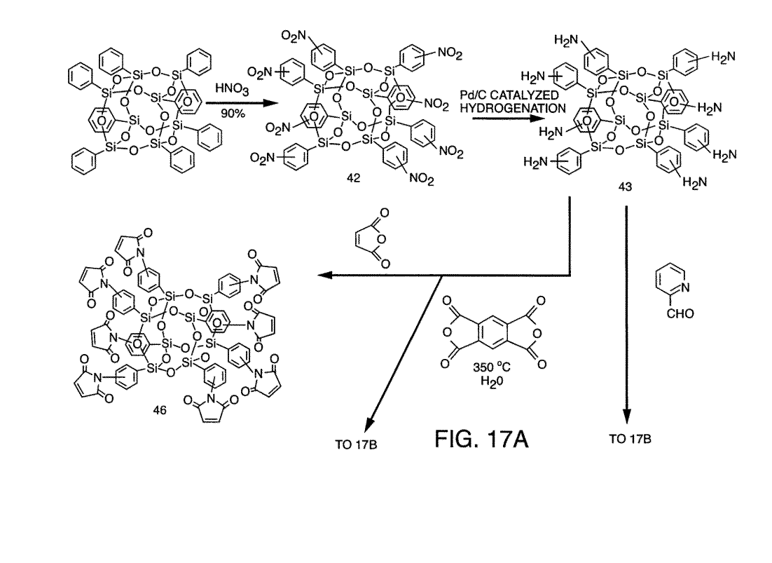

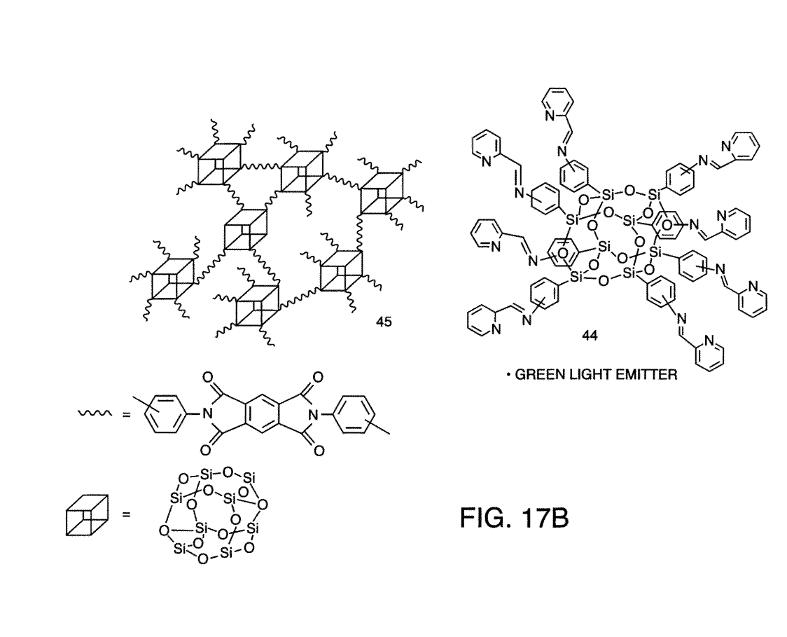

As illustrated in FIG. 17 after nitration of octaphenyl POSS 42, one may produce the octaaminophenyl POSS 43 by Pd/C-catalyzed hydrogenation of 42. Tamaki et al., JACS, 2001, 123, 12416-12417. One obtains a derivative, 44, by Schiff's base formation upon reaction of 43 with the ortho-carboxyaldehyde of pyridine. Furthermore, one uses the octaamino 43 with dialdehydes to make polyimide cross-linked networks. One reacts POSS 43 with maleic anhydride to make the octa-N-phenylmaleimide, 45, which could serve as a cross-linking agent in maleimide polymer chemistry.

### Siloxane Macromers

To design synthetic constructs that meet the combined structural, mechanical and biological requirements of viable bone grafts, we propose a class of star-shaped polymer building blocks (macromers) mechanically strengthened with inorganic nanoparticle cores and flanked with block copolymer arms (FIG. 20). The macromers are designed to promote the recruitment and adhesion of osteoprogenitor cells via cell adhesive RGD epitope, retain and release exogenous BMP-2/BMP-2/7 heterodiamer/RANKLNEGF to simultaneously trigger new bone formation and osteoclastic remodeling of the synthetic graft with vascular ingrowth, and template the nucleation and growth of HA in situ. The macromers can be further crosslinked to form stable bone grafts either prior to implantation or at the site of injection under physiological conditions. The graft is also designed to degrade overtime to allow eventual replacement by newly integrated bony tissue.

Integration of a bioactive synthetic graft with its tissue environment requires favorable cell-material interactions at the tissue-graft interface. Integrins link the intracellular cytoskeleton of cells with the extracellular matrix by recognizing the RGD motif. The covalent attachment of RGD peptide to material surfaces has proven to be an effective way to control cell adhesion to biomaterials including artificial tissue scaffolds. We present the RGD epitope at the surface of each macromer building block to promote the recruitment of osteoprogenitor cells and facilitate tissue penetration throughout the 3-dimensional scaffold upon crosslinking of the macromers.

The localized delivery of exogenous BMP-2/BMP-2/7 heterodiamer/RANKL/VEGF by the synthetic grafts provides for an efficient carrier of these biomolecules. Chemical modifications of growth factors to enhance their tissue specific retention have been attempted in the case of bone tissue repair. These approaches, however, typically involve multi-step bioconjugation chemistry to be performed to each protein target of interest and suffer from the inherent uncertainty of the protein bioactivity upon structural perturbations. In nature, anionic polysaccharides such as heparin are known for their inherent high affinity for basic growth factors including rhBMP-2 and VEGF. The favorable electrostatic interaction between the anionic matrix and the basic growth factors and cytokines can be recapitulated in the design of synthetic delivery vehicles of these proteins. Indeed, the concept of utilizing electrostatic interactions to improve the retention and release characteristics of proteins has been validated by a number of studies using naturally occurring hydrogels as growth factor delivery vehicles. For instance, hyaluronic acid and gelatin more effectively retain basic growth factors and release them in a more sustained manner. In our design, polymethacrylamides that are rich in sidechain carboxylates and with tunable polymer chain lengths (thus, adjustable affinity to the basic proteins) are grafted to each hybrid macromer to realize efficient retention and sustained local release of BMP-2/BMP-2/7 heterodiamer/RANKL/VEGF upon crosslinking. The negatively charged sidechain carboxylates also serve as crosslinking sites when the macromers are exposed to diisocyanate cross-linkers.

The incorporation of the HA-binding peptide identified by the combinatorial screening approach is designed to enhance the bonding affinity of the graft with its surrounding bony tissue, as well as to facilitate the graft-templated HA-mineralization in vivo. The in situ integrated HA minerals are expected to help sequester the ECM proteins (e.g. osteopontin and bone sialoprotein) secreted by osteoblasts via favorable binding of these proteins to the HA crystals. Preventing the secreted cytokines and growth factors from quickly diffusing away from synthetic scaffolds (thus maintaining their tissue-specific critical local concentrations) is an important consideration in the design of ECM mimetics.

Further, the macromers are designed to degrade over time to allow its eventual replacement by new bone. This is realized by the grafting of well-characterized biodegradable poly(rac-lactide) (PLA) segments to the POSS cores. Whereas the more crystalline packed poly(L-lactide) tend to degrade slowly, with degradation ranging from months to many years, the in vitro and in vivo hydrolysis of the amorphously packed poly(rac-lactide) is faster (with median degradations in a few months) due to faster water uptake. It is contemplated that the in vivo degradation of the graft will coordinate with the new bone ingrowth within the time scale of the normal fracture healing. However, a slower degradation rate and higher mechanical strength of the graft can be achieved by enhancing the L-lactide content of the PLA chains, or vise versa if the opposite effect is desired, via the stoichiometric control of the monomers during the ROP grafting.

Polyethylene glycol diisocyanates may be used to crosslink and stabilize the polar macromers by forming urethane linkages between the isocyanate functionality and the free carboxylates richly present in the growth factor retention domain. The length of each functional domains attached to the POSS core can be independently altered during the sequential assembly of the block copolymer segments. This feature allows for the optimization of the biodegradation rate, polarity, charge, aqueous solubility and viscosity of the star-shaped macromers. By adjusting the cross-linker length and crosslinking density, the growth factor release characteristics and the mechanical properties can be further optimized. Comparing to naturally occurring hydrogels and polysaccharides, synthetic scaffolds assembled from bottom up are characterized with better-controlled physical, mechanical and biological properties.

### EXPERIMENTAL

The following examples are provided in order to demonstrate and further illustrate certain preferred embodiments and aspects of the present invention and are not to be construed as limiting the scope thereof.

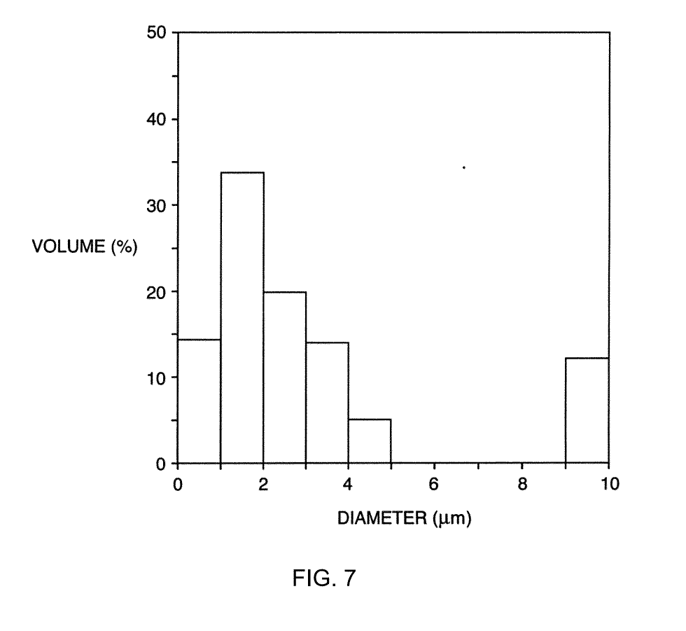

The radical inhibitors in the commercially available HEMA and EGDMA (Aldrich, Milwaukee, Wis.) were removed via distillation under reduced pressure and by passage through a 4 Å molecular sieve column prior to use, respectively. Polycrystalline HA powders were purchased from Alfa Aesar (Ward Hill, Mass.) and used as received. The calcined HA powders were obtained by treating the commercial polycrystalline HA at 1100° C. for 1 h. Prior to use, the calcined powders were ground in a planetary agatar mill for 2 h and then passed through a 38 μm sieve to remove larger agglomerates. The microstructures and size distributions of these HA particles are shown in FIG. 7. Cell culture media and supplements were purchased from Invitrogen (Carlsbad, Calif.) and the fetal bovine serum (FBS) was purchased from HyClone (Logan, Utah). All reagents for histochemistry were purchased from Sigma (St Louis, Mo.).

### Example I

### Preparation and Processing of the Flexbone Composites

The HA content of the FlexBone is defined as the weight percentage of the HA incorporated over the total weight of the HA, hydrogel monomer HEMA, and cross-linker ethylene glycol dimethacrylate (EGDMA) used in any given preparation. In a typical procedure, freshly distilled HEMA was mixed with EGDMA along with ethylene glycol, water and aqueous radical initiators ammonium persulfate (480 mg/mL) and sodium metasulfite (180 mg/mL) at a volume ratio of 100:2:55:0:5:5 (formulation 1), 100:2:20:35:10:10 (formulation 2), 100:2:35:20:5:5 (formulation 3), or 100:2:60:40:5:5 (formulation 4; applied to composites containing >50% HA only). Commercial HA or calcined HA powder was then added to the hydrogel mixture, thoroughly mixed by using a ceramic ball to break up the large agglomerates, and allowed to polymerize in a plastic syringe barrel to afford composites with HA contents varying from 30% to 70%. The resulting rubbery material was removed from the syringe barrel.

Elastomeric high-mineral content composites were cut into pieces and soaked in a large volume of water overnight before freeze-drying or undergoing solvent exchange with glycerol. The resulting composites are denoted as #Com/Cal-N-AP/FD, where # denotes the weight percentage of HA, Com for commercial HA, Cal for calcined HA, N for the type of hydrogel formulations (1, 2, 3 or 4), AP for as-prepared, and FD for freeze-dried. For instance, 70Cal-4-AP represents as-prepared FlexBone with 70% calcined HA that is formed using hydrogel formulation 4, whereas 40Com-3-FD represents freeze-dried FlexBone with 40% commercial polycrystalline HA that is formed using hydrogel formulation 3.

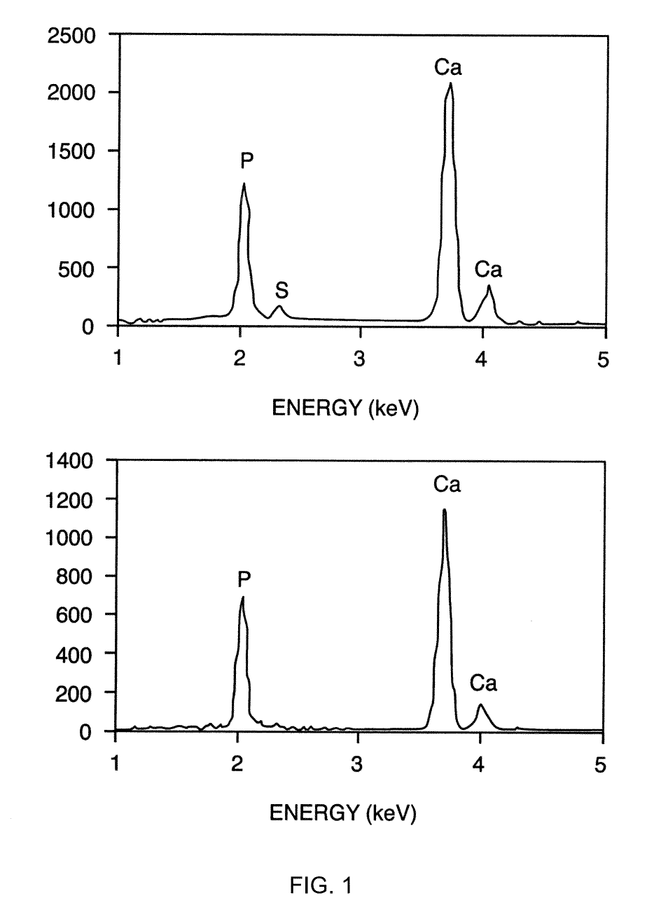

The composites produced by this method could be compressed or bent without fracturing, and be cut into desired shapes and sizes. While as-prepared FlexBone produced in ethylene glycol as the main solvent (formulation 1) remained highly elastic even after months of storage under ambient conditions, formulations with lower ethylene glycol-to-water ratios generated composites with reduced flexibility. The loss of water via evaporation during solidification or upon storage is likely to have contributed to the compromised elastomeric properties of FlexBone produced in low-glycerol content solvents. The as-prepared composites can undergo solvent exchange with water or other viscous solvents such as glycerol, or freeze-dried (after removal of ethylene glycol by exchanging with water) to afford materials with varied strength and stiffness. The residual radical initiators could be removed via solvent exchange. The S signal detected from the energy dispersive spectroscopy (EDS) performed on the cross-section of the composite, associated with the ammonium persulfate and sodium metasulfite trapped in as-prepared sample, disappeared upon freeze-drying following solvent exchange with water (FIG. 1). With FlexBone samples possessing very high HA content (>50%), complete exchange of ethylene glycol with water prior to freeze-drying was more difficult to achieve. In those cases, prolonged solvent exchange (up to several days) and repeated hydration/freeze-drying was required to completely remove residue radical initiators and ethylene glycol.

### Example II

### Microstructural Characterization and Compression Tests

The microstructures of the composites were characterized using environmental scanning electron microscopy (ESEM) on a Hitachi S-4300SEN microscope (Hitachi, Japan). The chamber pressure was kept at ˜35 Pa to avoid complete sample dehydration and surface charging during the observation. The chemical composition was analyzed using energy dispersive spectroscopy (EDS) (Noran System SIX, Thermoelectron, USA) attached to the ESEM.

Two types of HA powder were used: the commercial polycrystalline powder (Alfa Aesar, Ward Hill, Mass.) consisting of micrometer-sized loose aggregates of HA crystallites that are ˜100 nm (nanocrystals) in size and HA powder calcined at 1100° C. Calcined HA powder consisted of dense particles with a bimodal size distribution at the submicrometer scale (FIG. 7). Both types of HA powder were well distributed throughout the hydrogel network at all mineral contents examined, as indicated by SEM analysis. Examples of composites possessing 50% HA are shown in FIGS. 4A and 4B. Excellent mineral-gel integration was maintained upon freeze drying, suggesting strong adhesion at the organic-inorganic interface. In addition, no detectable mineral dissociation from the composites containing up to 70% HA was observed upon storage in water at 37° C. for more than one year, further supporting the strong mineral-gel integration.

SEM analysis further revealed that the freeze-dried composites containing 50% calcined HA versus 50% commercial HA display different structural changes in response to compressive stress. The cross section of the composite containing calcined HA particles (50Cal-3-FD) showed no distortion or delamination of the HA particles from the hydrogel phase even after being compressed >80%. SEM analysis of FlexBone containing commercial HA (50Com-3-FD) showed that the hydrogel-infiltrated HA nanocrystal aggregates had flattened upon compression into plywood-like structures. This structural reorganization was irreversible under stress levels on the order of several hundred megapascals, which far exceeding normal physiological loads.

Standard unconfined compression tests were performed to evaluate the compressive behavior of the hydrogels and the composites produced. Short cylindrical samples, nominally 3-6 mm in height and 4-7 mm in diameter, were cut from the bulk material using a razor blade. Full contacts of both surfaces with the rigid platens of the testing machine were examined to ensure that the cuts were parallel to each other. Testing was performed in ambient air on a high-capacity MTS servo-hydraulic mechanical testing machine (MTS Systems Corporation, Eden Prairie, Minn.) fitted with stiff, non-deforming platens. The samples were loaded under displacement control at a rate of ˜0.015 mm/s, while the corresponding loads and displacements were continuously monitored using the in-built load cell and linear variable displacement transducer (LVDT). Three samples were tested for each composition and the representative compressive force-strain curves were plotted. The mean stress±S.D. at the selected strains and compositions, calculated based on the measured surface area in direct contact with the platen, are summarized. To characterize the reversibility of the compressive behavior of as-prepared FlexBone samples at low to moderate strains, loading and unloading was repeated 3-5 times sequentially on the samples up to 40% strain. If after 3 sequential loading and unloading cycles, one observed energy dissipation, then the test was end. Otherwise, 2 more loading and unloading cycles were repeated.

Standard unconfined compression tests and SEM coupled with EDS were performed to characterize the quantitative compressive behavior and microstructural properties of the composites. Most freeze-dried FlexBone samples tested withstood compressive stress on the order of hundreds of megapascals without exhibiting brittle fractures. In comparison, PMMA-based bone/dental cements or poly(lactic acid)-HA composites reported in the literature exhibited brittle fracture at 50-150 MPa compressive loading. The representative compressive force-strain curves (loading curves) shown in FIG. 2A indicate that an as-prepared FlexBone composite containing 37% commercial polycrystalline HA powder (37Com-3-AP), prepared using formulation 3, underwent >80% strain and over 500 MPa stress without fracturing. By contrast, comparable pHEMA hydrogels lacking HA exhibited significantly decreased compressive strength. Freeze-dried composites were more rigid than the as-prepared sample, withstanding up to 600 MPa of stress while undergoing >80% compressive strain. Although small cracks were formed along the periphery of the freeze-dried composites under high compressive load (FIG. 2B), no fractures were observed across the bulk material.

The compressive strength of the composites was dependent on the mineral content. As shown in FIG. 3, the work under the compressive force-strain curve of a freeze-dried FlexBone possessing 48% commercial polycrystalline HA (48Com-3-FD) is greater than that of the sample containing 41% HA (41Com-3-FD), indicating that the higher-mineral content resulted in a stiffer, tougher, and stronger composite.

Compressive force-strain curves (FIG. 4A) and compressive stresses at selected strains (FIG. 4B) showed that the FlexBone composite containing 50% commercial polycrystalline HA powder (50Com-3-FD) was stiffer, stronger, and tougher than the material containing 50% calcined powder (50Cal-3-FD). Smaller error bars were obtained with the compressive stress measurements of 50Com-3-FD comparing to those obtained with 50Cal-3-FD. (FIG. 4B). This difference may reflect better integration of the organic and inorganic components within the spongy aggregates of HA nanocrystals in the commercial powder.

It was identified that despite the high mineral contents, most as-prepared composites were able to undergo multiple compressions up to 20%-40% strains with excellent shape recovery. The recovery of freeze-dried composites from compressive loadings or as-prepared composites from high strains (e.g. >40%), on the other hand, was not as good. The reversibility of the compressive behavior of as-prepared FlexBone composites at moderate strain (<40%, with mechanical load in the order of a few megapascals) was thus characterized with repetitive compressive loading and unloading. As shown in FIG. 5, as-prepared FlexBone composites possessing 40% (40Cal-3-AP) or 70% calcined HA powder (70Cal-4-AP) were both able to recover from repetitive moderate strains, with minimal energy dissipation (area between the loading and unloading curves) observed within the tested strain levels. Similarly, as-prepared FlexBone containing commercial HA powder also displayed reversible compressive behavior at moderate compressive strains. FlexBone 37Com-3-AP recovers from a mild compressive load at 2.6 MPa. In comparison, the peak contact stresses in natural human joints during light to moderate activity typically range from 0.5-6 MPa by most in vitro measurements, and up to 18 MPa by in vivo measurements.

### Example III

### In Vivo Resorption of Flexbone Composites and their Ability to Support Osteogenic Differentiation

All animal procedures were conducted in accordance with the principles and procedures approved by the University of Massachusetts Medical School Animal Care and Use Committee. Rat bone marrow stromal cells BMSC were isolated from long bones of 4-week old male Charles River SD strain rats. Marrow was flushed from the femur with a syringe. After lysing red blood cells with sterile water, the marrow cells were centrifuged and resuspended in minimum essential medium (MEM) supplemented with 20% FBS, 0.2% penicillin-streptomycin and 1% L-glutamine, and passed through a sterile metal filter. Cells were expanded on tissue culture plates (10 million cells per 100-mm plate) with media changed every other day before being lifted off on day 4 for plating on FlexBone.

FlexBone composites were subcutaneously implanted with and without pre-seeded BMSC in rats. Thin half discs (7 mm in diameter, 1 mm in thickness) of FlexBone containing 40% calcined HA (40Cal-3-AP) or 40% commercial HA (40Com-3-AP) were sterilized in 70% ethanol, re-equilibrated with sterile water before being seeded with BMSC and used for subcutaneous implantation in rats. Fifty microliters of BMSC suspension (in culture media described above) was loaded on the surface of thin disks of FlexBone to reach 5,000-cells/cm² or 20,000-cells/cm² seeding density. The cell-seeded FlexBone was incubated at 37° C. in humidified environment with 5% CO₂ without additional media for 6 hours to allow cell attachment to the FlexBone substrate. Additional media were then added and the cells were cultured on the substrates for 2 days before being used for implantation. Four sets of samples were used for each FlexBone composition and cell seeding treatment. Thin discs of FlexBone without pre-seeded BMSC were also used for implantation as controls.

Rats were anesthetized by intraperitoneal (IP) injection of ketamine/xylazine (50 mg/5 mg per kg). They were shaved and swabbed with betadine before two 0.25 inch bilateral skin incisions were made over the rib cage for insertion of the FlexBone discs with and without pre-seeded BMSC. The skin was closed with surgical staples and buprenorphine (0.02 mg/kg) was given subcutaneously. The rats were sacrificed by CO₂ inhalation and cervical dislocation at day 14 and day 28 for the retrieval of FlexBone. After removing the fibrous tissue encapsulation, the retrieved FlexBone was fixed in 4% paraformaldehyde (0.1 M phosphate buffer, pH 7.4) for 5 h at 4° C. before being analyzed by SEM, XRD, and histology.

To test the cytocompatibility and the in vivo resorption of FlexBone, we seeded composites containing 40% calcined or commercial HA (equilibrated with water prior to cell seeding to remove any residue radical initiators and ethylene glycol) with bone marrow stromal cells (BMSCs) isolated from rat femur, and implanted them subcutaneously (SC) in 4-week old male Charles River SD strain rats. The composites were retrieved at 2 and 4 weeks, with a degree of fibrous tissue encapsulation observed in all cases. After removing the fibrous tissue, the morphology and mineral phase of the retrieved implant were examined by SEM and X-ray powder diffraction. Although little change in shape or size of the retrieved FlexBone was observed visually, reflecting the non-degradable nature of the hydrogel scaffold that defines the overall shape of the composite, we observed an increase of surface microporosity of the composite over time. Both composites with and without pre-seeded BMSC showed significantly roughened and more porous surfaces after being implanted subcutaneously in rats for 2 and 4 weeks as compared to the surface of the composite prior to implantation. This is presumably due to the slow dissolution of the mineral component in vivo since the substrates incubated under standard cell culture conditions did not undergo a similar increase of surface porosity.

### Example V

### X-Ray Powder Diffraction (XRD)

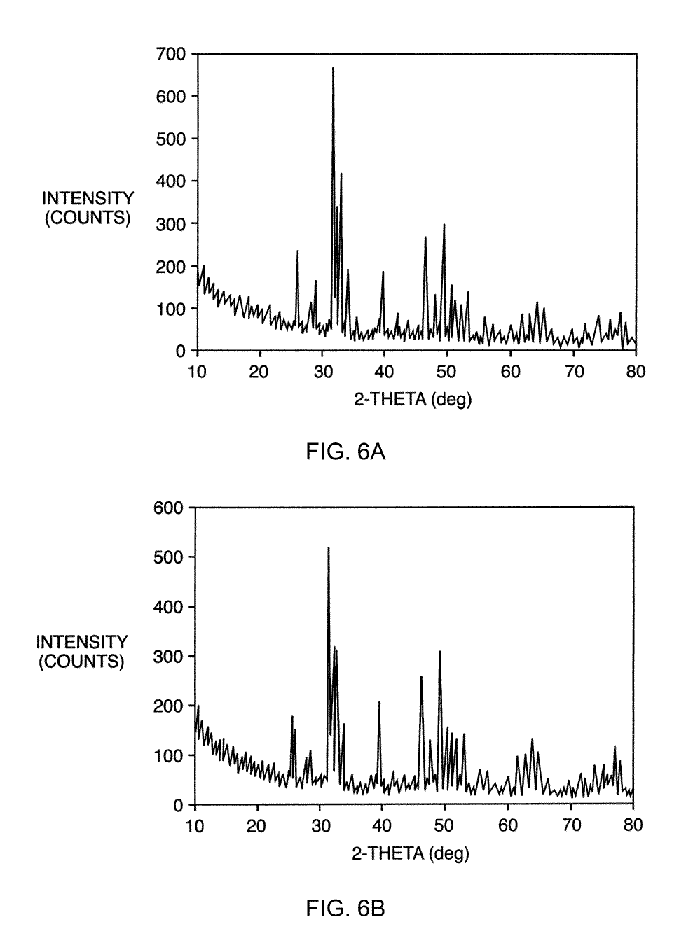

The crystalline phases of the mineral in the FlexBone composites before and after subcutaneous implantation in rats were evaluated by XRD with a Siemens D500 instrument using Cu K_{α} radiation. The phases were identified by matching the diffraction peaks to the JCPDS files. XRD analyses performed with the 40Cal-3-AP composites before (FIG. 6A) and after implantation (FIG. 6B) revealed little changes in diffraction patterns, with the typical reflections of both matching those of synthetic crystalline HA standards. No qualitative difference in terms of the in vivo dissolution behavior of the composites containing calcined versus commercial HA was observed.

### Example VI

### Histochemical Staining of Explanted Flexbone for Alkaline Phosphatase (ALP) Activity

The 4% paraformaldehyde-fixed FlexBone explants of Example IV were equilibrated in cacodylic buffer overnight, then in 30% sucrose solution (pH 7.3) for 2 days before being frozen-sectioned on a Bright Cryostat (Model OTF; Bright Instrument Ltd., Huntigdon, UK). Frozen-sectioning was repeated until reaching the depth of 100-200-μm away from the surface where the BMSC were initially seeded. The 12-μm frozen sections were held on adhesive slides using frozen sectioning tape for UV cross-linking (˜1 sec). Histological staining for ALP activity, a marker of osteogenic differentiation, was performed. The frozen sections of FlexBone explants were incubated with 1.5 mM naphthol-As-Mx phosphate disodium salt, 0.1% Fast Red and 2.7% DMF (v/v) in 0.1M Tris acid maleate buffer (pH 8.4) for 30 min, and the positive stains (in red) were detected by light microscopy.

To determine whether the composites can support the osteogenic differentiation of BMSC in vivo, the explanted composites with pre-seeded BMSC were stained histochemically for alkaline phosphatase (ALP) activity, a marker for osteogenic differentiation. To avoid the harsh paraffin embedding conditions that may compromise ALP enzymatic activity, frozen sectioning was performed on the explants prior to ALP staining. As shown by optical microscopy images (not shown), ALP activity (indicated by red stains) was detected 14 days post-implantation on the periphery of the calcined HA-containing composite pre-seeded with 5000-cells/cm² BMSC. More extensive ALP activity was detected 28 days after the implantation deeper inside the FlexBone pre-seeded with 20,000-cells/cm² BMSC. Similar results were observed with the composites containing 40% commercial HA. These data suggest that the BMSC attached to the FlexBone are able to migrate through the thin composite discs (note that the frozen sections were obtained 100-200 micrometers away from the surface where the BMSC were initially seeded) and undergo osteoblastic differentiation.

### Example VII

### Surgical Procedure of Flexbone with Hydroxyapatite (HA) and Beta-Tricalcium Phosphate (TCP)

FlexBone composites containing 60% HA, 45% HA-15% TCP, 30% HA-30% TCP, 15% HA-45% TCP, and 60% TCP were prepared as described in Example I in syringe barrels with 3-mm inner diameters. The mineral content, in weight percentage, is defined as the weight of HA/TCP divided by the combined weight of HA/TCP, HEMA and cross-linker EGDMA. The as-prepared composites were equilibrated in water for 24 h, with frequent changes of fresh water, to remove residue radical initiators and unpolymerized monomers. The composites were then cut into segments of 5.5-mm in length before they were freeze-dried. The composite grafts were re-hydrated in saline 30 min prior to implantation, and their final lengths are optimized by a surgical knife to match with the segmental defects before being inserted to the site of femoral defects.

A male Charles River Sprague-Dawley strain rat (290±10 g) was anesthetized by 5% isoflurane and 2% oxygen in an induction chamber before its left hind leg was shaved bilaterally and swabbed with betadine. The rat was maintained by 2% isoflurane and 2% oxygen throughout the surgery via a rodent nose mask on a heated sterile surgical area. An anterior incision was made with the convexity between the base of the rat tail and the knee. The shaft of the femur was exposed by blunt dissection between the vastus lateralis and the hamstring muscles. A self-retaining retractor was used for exposure of the femur. The soft tissue of the femur was cleaned by a bone elevator.

A radio-transparent polyetheretherketone (PEEK) plate with 4 pre-drilled holes (designed and manufactured in Steve Goldstein's laboratory at the University of Michigan) was placed over the rat femur antero-laterally. The design features an elevation in the middle of the plate that permits easier removal of bone and subsequent insertion of grafts. Guided by one of the center holes of the PEEK plate, a Dremel tool attached with a 1/32″ drill bit (Dremel USA, Part #660 with Collet) drilled transversely through the femur before a self-tapping cortical screw (Morris Company, Part # FF00CE250) was applied immediately after. The process was repeated from the center towards both ends of the PEEK plate until the plate was securely held to the femur by all 4 cortical screws. A Hall oscillating saw, adapted with two parallel blades separated by a spacer block, was used to create 5-mm segmental defects on the rat femur directly under the plate elevation. The segmental bone piece and debris were removed by irrigation with saline. A hydrated FlexBone graft of similar size and shape to the removed bone piece was tight-fit into the segmental defect before the vastus lateralis was approximated and closed with the hamstring muscle using 4-0 Vicryl sutures. The fascia was also closed with 4-0 Vicryl sutures before the outer incision was closed with surgical staples. Local infusion of Bupivacaine (0.125% solution) was applied. The same procedure was repeated on the right femur of the rat, with or without (serving as the control) the insertion of a synthetic graft containing a different ceramic composition. Buprenorphine (0.04 mg/kg SC) and Cefazolin (20 mg/kg) were administered subcutaneously as analgesics and antibiotics immediately after the surgery. The rat was then allowed to recover off the rodent ventilation machine and returned to the cage. The rats could usually regain strength to move around within 30 min to 1 h post-operation. Buprenorphine (0.02 mg/kg SC) was given twice a day for two more days and Cefazolin was given once more on the second day after the surgery. Surgical staples were removed after 14 days. We have not observed any incidents of infection using pHEMA-HA/TCP composite grafts in combination with the plate fixation technique.

X-ray radiographs were taken both post-operatively and biweekly thereafter to confirm the proper positioning of the graft and to follow its mineral content resorption over time until the animal is sacrificed at various time points (e.g. 4 weeks and 8 weeks post-operation) by CO₂ inhalation and cervical dislocation. A pHEMA-ceramic graft containing 15% HA and 45% TCP (by weight) was snugly fit into the segmental defect and remained in place 2 weeks after the surgery despite the active movements of the rat. Key features of the healing of segmental defects include the formation of a mineralized callus completely bridging the segmental defects, abundant neovascularization, and extensive resorption of bone graft. We observed partial callus formation bridging over 4-week explants in all graft compositions examined, and X-ray radiography indicated partial calcification of the callus. These data suggest that with a high content of osteoconductive minerals, FlexBone is capable of inducing graft healing.

Osteoclast formation was monitored by staining for tartrate-resistant acid phosphatase (TRAP), which is a marker enzyme of osteoclasts. More TRAP positive stains were detected at week 8 than at week 4. However, the overall resorption of the FlexBone grafts was limited, underscoring a preference for a biodegradable organic matrix of the graft and the exogenous supply of growth factors and cytokines to expedite the graft remodeling.

### Example VIII

### In Vitro Bioactivity of Graphs Pre-Absorbed with rhBMP-2, rmRANKL, and rhVEGF165

Grafts (5×5×1 mm, FlexBone 25% HA-25% TCP) were pre-absorbed with varying amounts of growth factors and cytokines to provide an exogenous supply for remodeling. Grafts loaded with growth factors rhBMP-2, rmRANKL, and rhVEGF165 were analyzed at the respective preferred doses. The preferred loading dose of RANKL (10 ng/FlexBone graft) was determined by the osteoclastic differentiation of macrophage RAW264.7, induced by the RANKL released from the graft as indicated by positive TRAP stains (purple) of multinucleated cells on Day 6. Unlike the “no graft” control, where 5 ng RANKL needed to be supplemented to the culture every other day in order to induce the differentiation, FlexBone released the RANKL in a sustained manner without the need for additional supplement. In contrast, without continued supplement of RANKL, pHEMA graft loaded with same amount of RANKL did not induce the osteoclastic differentiation, suggesting that the HA/TCP component plays a role in achieving the balance between sequestering and releasing RANKL. The preferred loading dose of rhBMP-2/7 heterodimer (40 ng/FlexBone graft) was determined by the osteogenic differentiation of C2C12 cells induced by the BMP-2/7 released from the graft in culture. We observed localized release of BMP-2/7 from the graft as indicated by the positive ALP stains localized around the graft by day 3. Finally, the preferred loading dose of rhVEGF165 in stimulating the proliferation of human vascular endothelial cells in culture was determined to be 5 ng/graft.

### Example IX

### Grafts Absorbed with Growth Factors for Surgical Implantation

Polymeric or polymer-HA/TCP composite grafts fabricated in a syringe barrel or plastic tubing (2-3 mm inner diameter) are cut into segments that are 5.5-mm in length, washed with water to remove residue, and freeze-dried the day before the surgery. Three holes along and perpendicular to the axis of the freeze-dried graft are drilled using a Dremel tool attached with a 1/16″ drill bit to facilitate the migration of bone marrow cells throughout the graft upon implantation. The freeze-dried grafts are loaded with the preferred doses of BMP-2 or BMP-2/VEGF/RANKL combination regimen in the maximal volume of aqueous buffer, as determined from the swelling ratio of the grafts, 1 h prior to implantation and kept in a humidified incubator at 37° C. The grafts without growth factor loading and the pHEMA control are equilibrated in saline in a similar fashion.

Prior to implantation to rat femoral defects, pre-drilling the graph with a hole that facilitates bone marrow cell migration throughout the graft. This method significantly enhanced the amount of new bone formation in the drill hole area facilitating cellular and new bone infiltration to materials that are not highly porous to start with, such as FlexBone.

Grafts of FlexBone (25% HA-25% TCP) absorbed with 40-ng rhBMP-2/7, 10-ng rmRANKL+5-ng rhVEGF165, or 40-ng rhBMP-2/7+10-ng rmRANKL+5-ng rhVEGF165 were press-fitted in 5-mm rat femoral defect sites, along with autograft control, pHEMA control and FlexBone control without growth factors. A total of 24 rats (N=4) were used to examine the graft healing at 4 and 8 weeks to elucidate the role of marrow access and exogenous growth factors in facilitating graft healing. Radiography follow-ups showed only <10% of the grafts were dislocated 2 weeks post-op, suggesting that the pre-drilled holes did not compromise the structural stability of the grafts. Substantial callus formation was observed by week 2 with the FlexBone graft containing a combination of 40-ng rhBMP-2/7+10-ng rmRANKL+5-ng rhVEGF165, suggesting that these exogenous growth factors and cytokines accelerate graft healing.

### Example X

### Hydrolytic Degradation Behavior of Urethane-Crosslinked Macromers

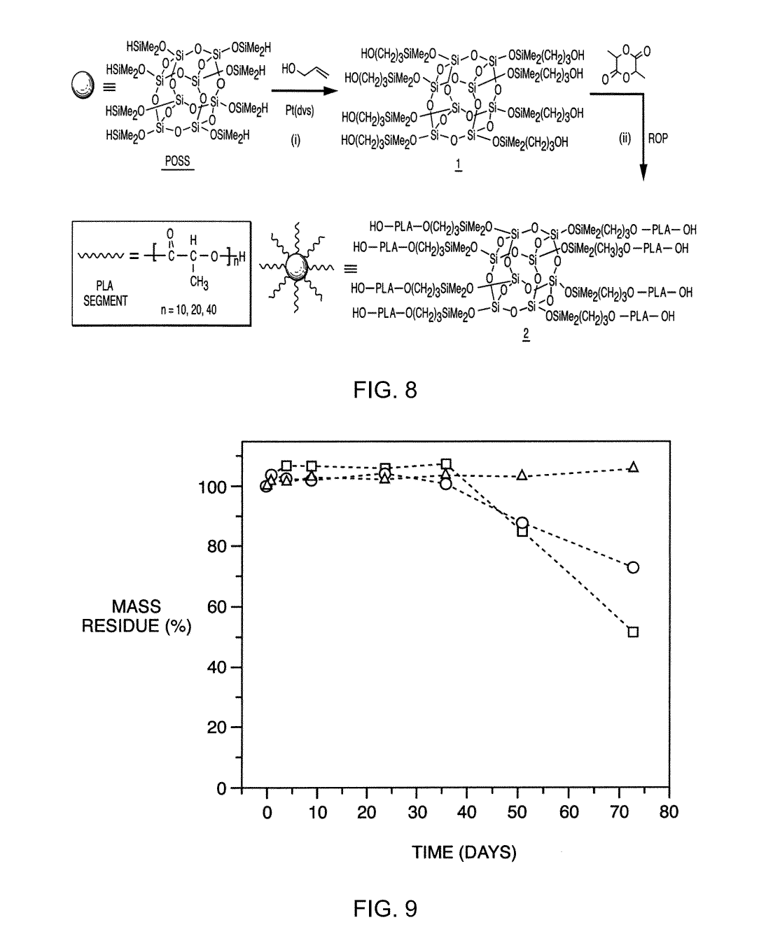

Urethane-crosslinked macromers with siloxane cores substituted with polylactides are described in U.S. Provisional Patent Application No. 60/925,329, filed Apr. 19, 2007. The hydrolytic degradation behavior of urethane-crosslinked macromer 2, POSS-(PLAₙ)₈, was examined in phosphate buffer saline (PBS, pH 7.4) at 37° C. over a course of 3 months (FIG. 8). The extent of in vitro degradation as a function of the polyester (PLA) chain lengths was monitored as the weight loss of the corresponding grafts over time (FIG. 9). As expected, the crosslinked macromers with the shortest PLA chain length (n=10) led to the fastest degradation, losing 50% of its mass in 73 days, whereas no significant mass loss was detected by 73 days with the crosslinked macromers containing much longer PLA chains (n=40). SEM micrographs confirmed that the grafts with shorter PLA chains (n=10, 20) degraded into highly porous materials by day 73 whereas little degradation was detected for the graft with longer PLA chain (n=40).

### Example XI

### Synthesis of Macromer CTA and the Grafting of pHEMA by Raft

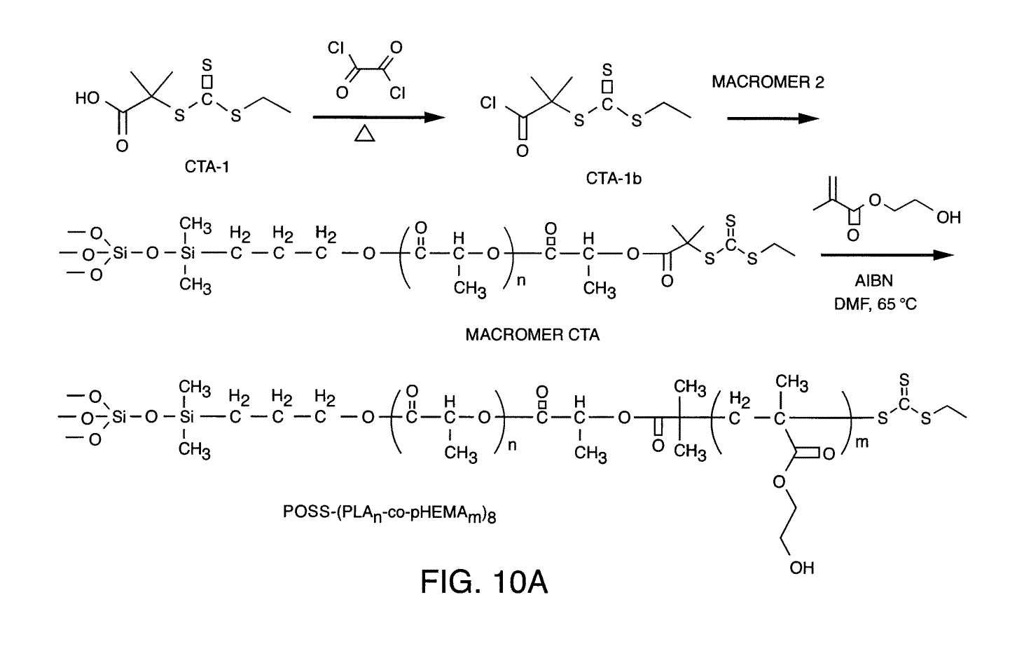

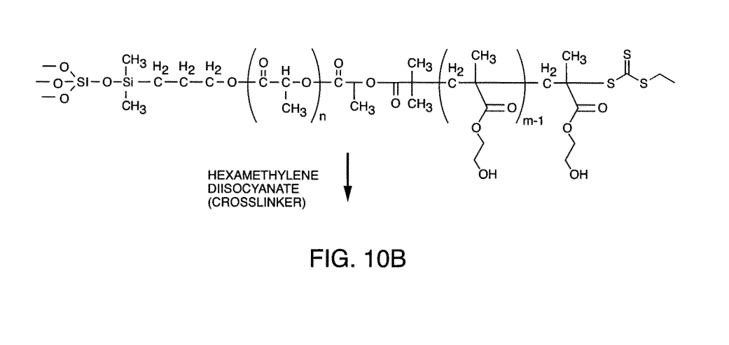

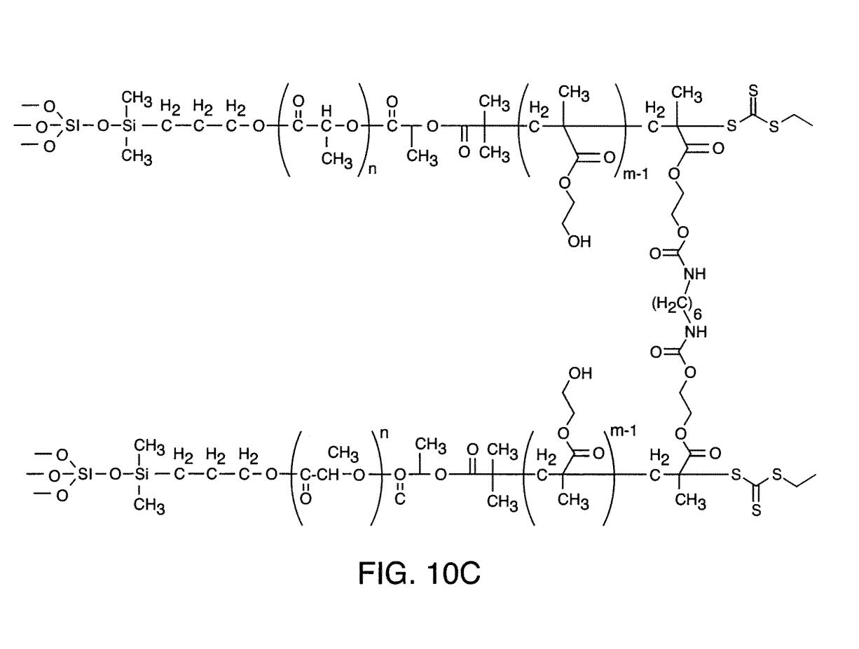

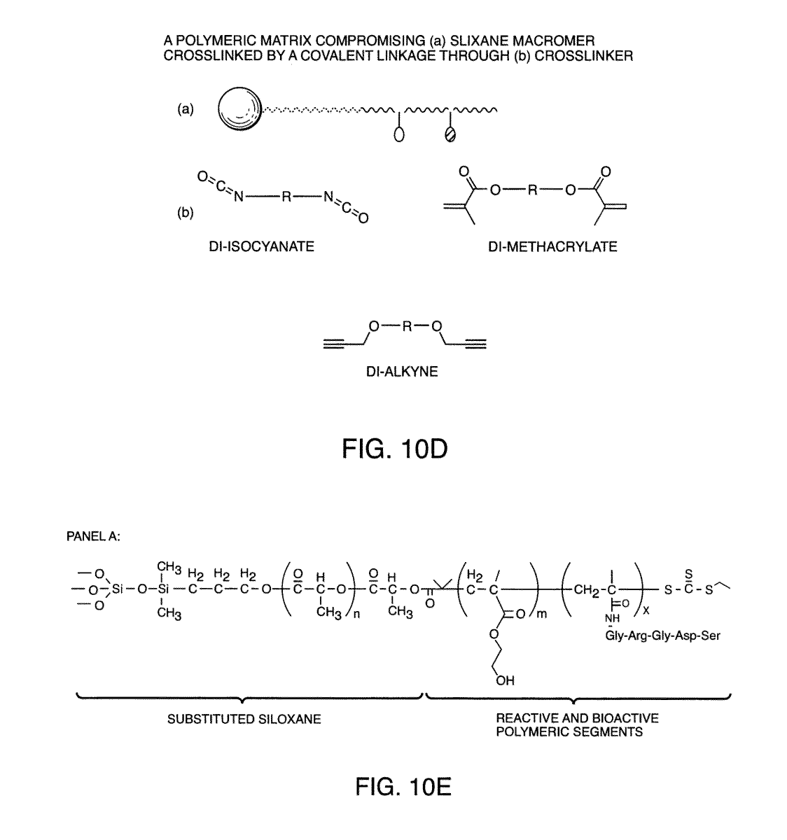

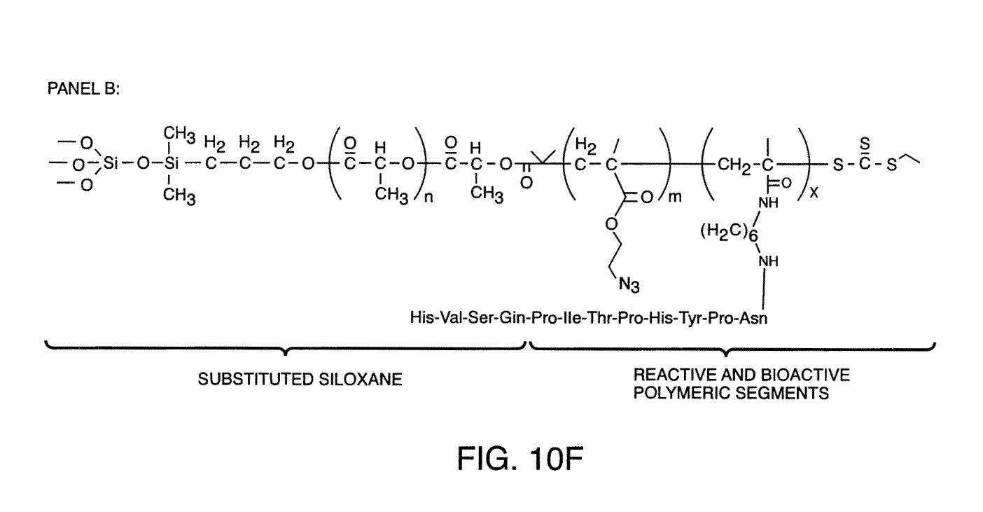

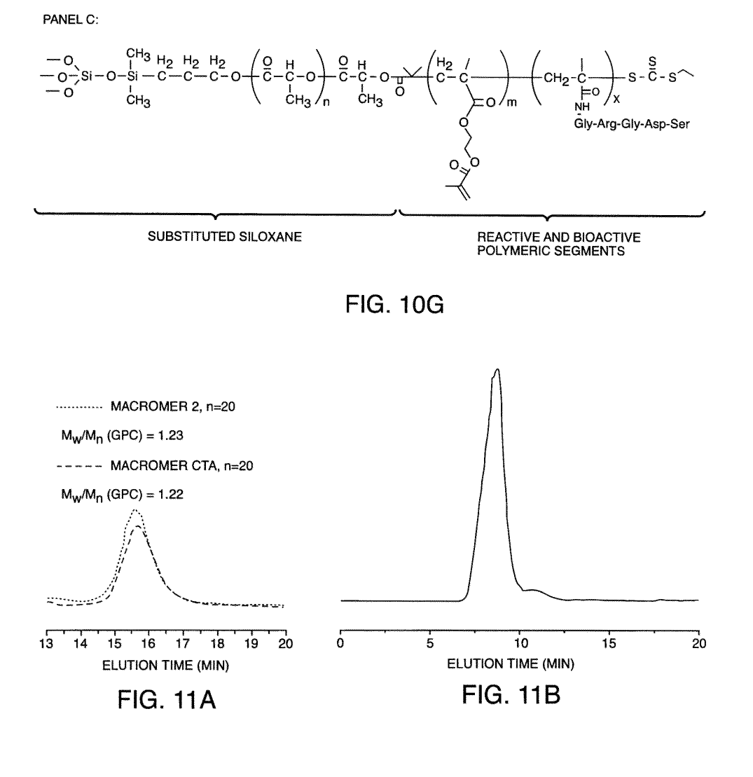

Trithiocarbonate and dithioester chain transfer agents (CTAs) were synthesized as provided in Mitsukami et al., Macromolecules 2001, 34, 2248-2256 and Convertine et al., Macromolecules 2006, 39, 1724-1730. The attachment of the trithiocarbonate chain transfer agent CTA-1, via the active acyl chloride intermediate, to the PLA termini of macromer 2 was accomplished in 92% yield (FIG. 10). Briefly, oxalyl chloride (1.455 g, 11.46 mmol) was reacted with CTA-1 (0.4662 g, 2.078 mmol) under N₂ for 2 h at room temperature and then 3 h at 55° C. The volatile component was removed under vacuum before macromer 2 (n=20, M_{w}/Mₙ=1.23, 0.5695 g, 0.039 mmol) in 15 mL THF was added. The reaction was allowed to proceed at 55° C. for 12 h before the volatile was removed by distillation. The resulting red oil was dissolved in 30 mL ethyl acetate, washed with 100 mL saturated NaHCO₃ aq. solution, dried with anhydrous MgSO₄, and precipitated in 100 mL hexane. The yellow solid was further purified by solvation in THF and precipitating in hexane three times. Drying under vacuum at 40° C. yielded spectroscopically pure macromer CTA (n=20, 0.5308 g, 92%). GPC characterization confirmed that the narrow molecular weight distribution (M_{W}/Mₙ=1.22) was retained upon the attachment of CTA to the macromer (FIG. 11).

The efficiency for the macromer CTA to initiate reversible addition fragmentation chain transfer (RAFT) polymerization was first investigated by grafting 2-hydroxyethyl methacrylate (HEMA) to each arm of the macromer. A solution of macromer CTA (n=20, PDI=1.22, 161.0 mg, 0.01 mM), AIBN (3.3 mg, 0.02 mM), HEMA (2.080 g, 16.0 mM) in 5 mL DMF was placed in a 25-mL Schlenk flask, degassed with three freeze-evacuate-thaw cycles, and reacted at 65° C. under N₂ for 10 h. The reaction mixture was precipitated in cold ethyl ether to yield a yellow solid, which was further purified by dissolving in DMF and precipitating in ethyl ether 3 times to give the final product (1.3 g, 65%). GPC characterization revealed a narrow molecular weight distribution (M_{w}/Mₙ=1.34), indicating the achievement of a well-controlled RAFT initiated by the macromer CTA. ¹H NMR data suggested a 222,000 molecular weight for the star-shaped polymer, correlating to an average of 200 repeating units in each grafted pHEMA arm.

### Example XII

### Crosslinking of Macromers

One functionalizes commercially available poly(ethylene glycol) (PEG, 1 and 5 kD) with isocyanate on both ends by reacting PEG with isophorone diisocyanate in 1,1,1-trichloroethane at elevated temperature in the presence of catalytic amount of dibutyltin dilaurate (FIG. 22). One purifies the PEG-diisocyanate cross-linkers by precipitation in chloroform/petroleum ether. One obtains different graft porosity and strength by using small molecule diisocyanates or PEG-diisocyanates with varying molecular weights (e.g. 1-5 kD) and crosslinking density (1, 2, 4 eq. PEG-diisocyanate per polymer arm, or 8, 16, 32 eq. PEG-diisocyanate per macromer).

One mixes dichloromethane solution of macromers (0.1 g/ml) and hexamethylene diisocyanate or PEG-diisocyanate (1 eq.) at room temperature for 15 min before being cast into molds to form films or bulk materials of desired shapes. One dries the solution under N₂ prior to covalent crosslinking at 80° C. for 48 h to form crosslinked polyester-urethane. The residue volatile components were removed in a vacuum oven at 70° C.

### Example XIII

### Crosslinking of Macromers in the Presence of Calcium Phosphate Aggregates

One obtains POSS-(PLAₙ)₈ or POSS-(PLAₙ-co-pHEMAₘ)₈, terminated with trithiocarbonate and dithioester or acrylates containing hydroxyl side chains (FIG. 12). One adds these macromers using appropriately modified methods as described in Example Ito provide a macromer-containing polymer aggregate composite.

### Example XIV

### Synthesis of Functional Methacrylamide Monomers

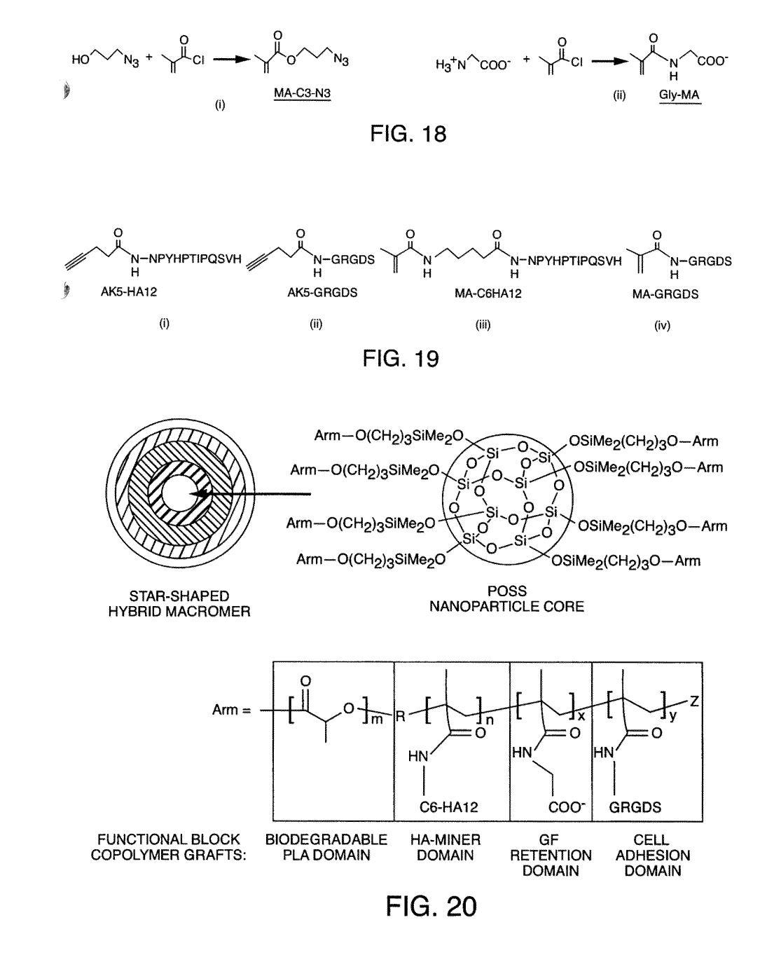

Two methacrylamides containing azido side chain (for click chemistry) and glycine side chain (for retaining growth factors) were prepared (FIG. 18). One functionalizes them to produce the corresponding macromer as provided in Examples 10 and 11. The synthesis of Gly-MA was achieved by coupling the N-terminus of glycine with methacryloyl chloride.

3-Azidopropan-1-ol: Sodium azide (3.92 g, 60.0 mmol) and 3-Bromo-1-propanol (5.00 g, 36.0 mmol) were dissolved in a mixture of acetone (60 mL) and water (12 mL), and refluxed at 75° C. for 10 h. After removing acetone under vacuum, 40 mL of water was added. The solution was extracted with 50 mL of ethyl ether 3 times. The ether phase was dried by anhydrous MgSO₄ and the solvent was removed by rotary evaporation, resulting in 3.00 g colorless oil (yield˜83%).

3-Azidopropyl methacrylate (MA-C3-N3): 3-Azidopropan-1-ol (1.010 g, 100.0 mmol) and triethylamine (1.220 g, 120.0 mmol) were mixed with 10 mL dichloromethane in an ice bath. Methacryloyl chloride (1.144 g, 110.0 mmol) was slowly added by a syringe in 30 min. The reaction was allowed to proceed in ice bath for 1 h before being warmed to room temperature and continued for another 2 h. After removing the insoluble salt by filtration, the filtrate was washed with 50 mL saturated NaHCO₃ aqueous solution 3 times. The organic phase was dried by anhydrous MgSO₄ and concentrated by rotary evaporation. The crude product was purification by flash chromatography (silica gel 60 Å, 70-230 mesh, ethyl acetate/hexane=1:7), resulting in 1.2 g colorless oil (˜70% yield).

### Example XV

### Functionalization of Cell Adhesive and HA-Binding Peptides

To attach the integrin-binding RGD epitope and the HA-nucleating ligand to the synthetic grafts, these peptides need to functionalized with proper reactive sites to accommodate the proposed bioconjugation chemistry. Using standard Fmoc chemistry for SPPS, we prepared the HA-12 peptide extended with a hexanoic acid linker (C6-HA12), the cell adhesive peptide GRGDS, and the alkynyl peptides AK5-HA12 and AK5-GRGDS (FIG. 19). The 6-carbon linker on the N-terminus of the HA-12 is designed to minimize the conformational perturbation of the peptide upon its covalent attachment to the macromer, ensuring the maintenance of its HA-nucleating capacity. In addition, methacrylamido group was attached to the N-terminus of the peptides, via the reaction of C6-HA12 and GRGDS with methacryloyl chloride in THF-H₂O (pH 8) to form MA-C6-HA12 and MA-GRGDS, respectively. The methacrylamido and alkynyl groups are introduced to allow the covalent coupling of these peptides to the star-shaped macromers. All crude peptides (60-70% purity) were characterized by mass spectrometry, with detected molecular weights matching with their theoretical values. These peptides will be further purified prior to use by HPLC on a preparative reversed phase (C18) column using acetonitrile-water (0.1% trifluoroacetic acid) as mobile phase.

### Example XVI

### Polymer Scaffold Design

Polyhedral oligomeric silsesquioxane (POSS) nanoparticles are designed as the structural and mechanical anchors for grafting multiple functional polymer domains to form the star-shaped macromers. After attaching the biodegradable PLA chains to the POSS core via ROP, an HA-nucleation domain containing the HA-binding peptide (HA-12), a negatively charged polymethacrylamide growth factor retention domain and a cell adhesion domain containing the integrin-binding Arg-Gly-Asp (RGD) epitope are sequentially grafted via RAFT polymerization. The R and Z groups depicted in FIG. 19 are the fragments of the chain transfer agent (CTA) attached to the macromer for initiating the RAFT. he POSS nanoparticle cores do not affect the radio-transparency of the hybrid polymer grafts, allowing for non-invasive tracking of the osteointegration of the polymer grafts by X-ray radiography.

### Example XVII

### Monomer Synthesis and Preparation of Star-Shaped Macromers

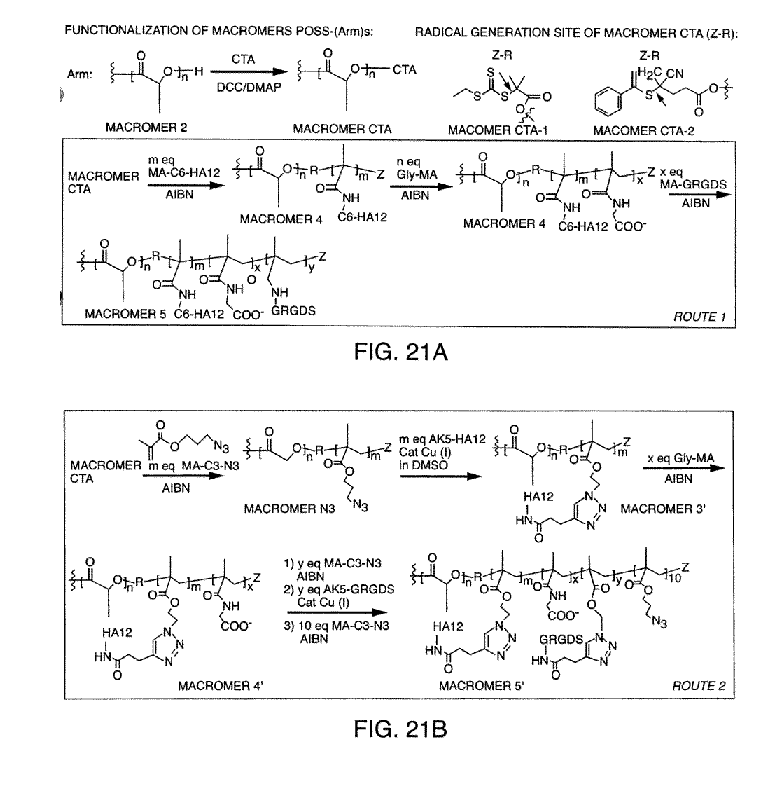

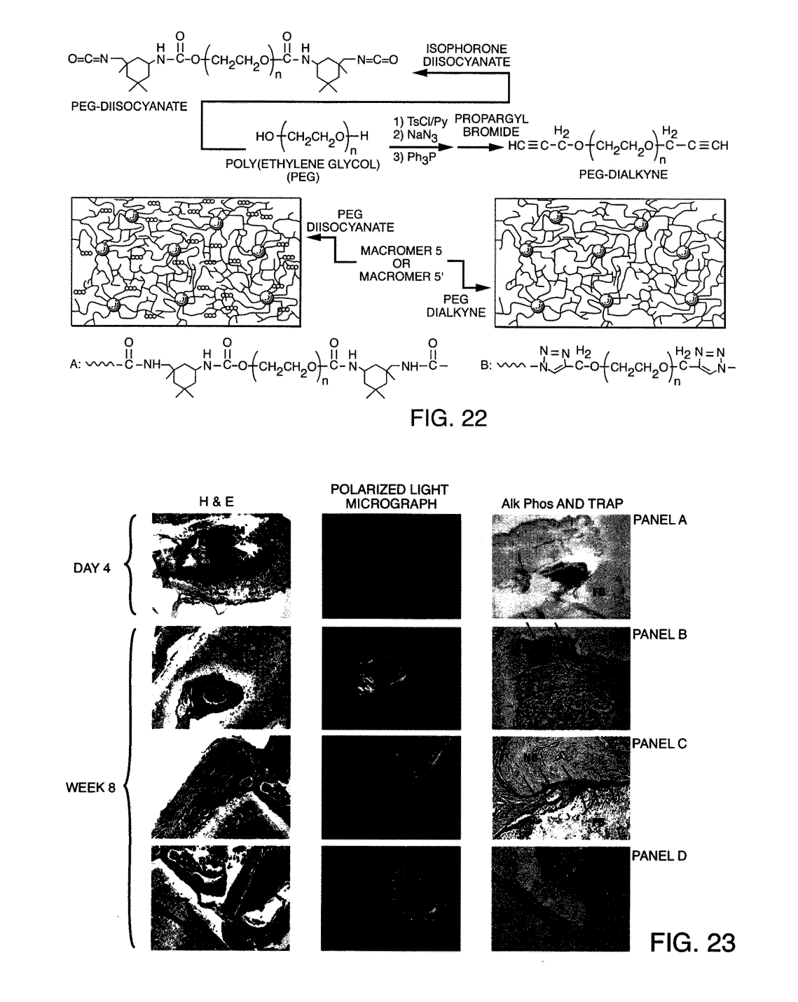

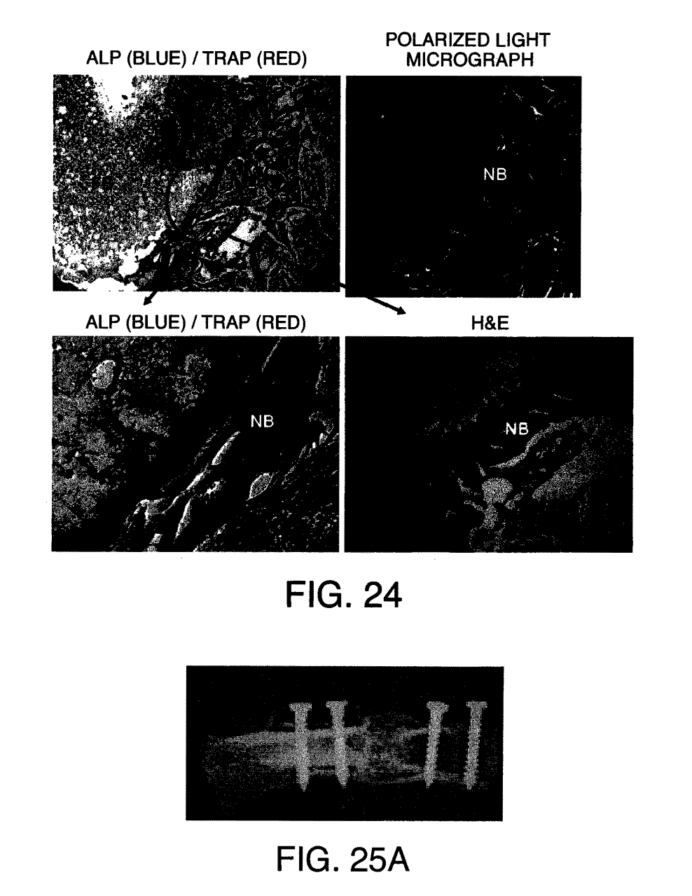

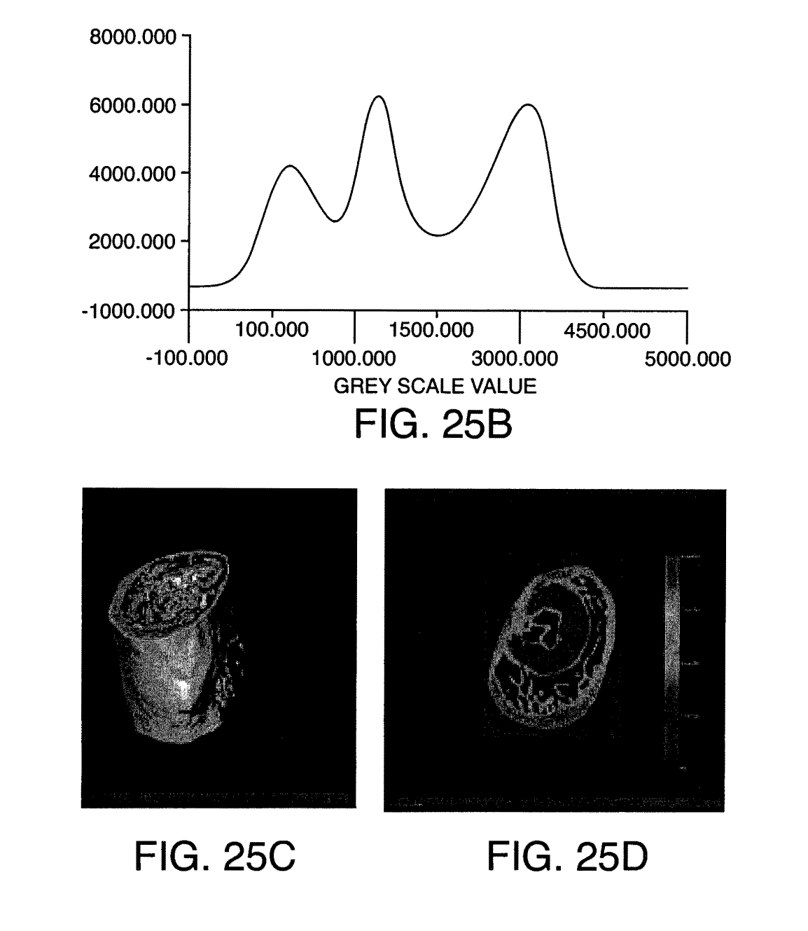

To initiate the RAFT, one covalently attaches previously prepared chain transfer agents CTA-1 and CTA-2 to the terminal hydroxyls of macromer 2 via esterification under the activation of 1,3-dicyclohexylcarbodiimide (DCC) and 4-(dimethylamino)pyridine (DMAP) (FIG. 21). The resulting macromer CTAs can generate benzyl or tertiary carbon radicals along the cleavage site (FIG. 21, top right), initiating subsequent RAFT grafting of polar polymer segments to the R fragment, capping the polymers with the Z fragment. By preparing both trithiocarbonate macromer CTA-1 and dithioester macromer CTA-2, one has the opportunity to choose the more efficient initiator for the subsequent RAFT. Following appropriately modified conditions as described in Covertine et al., Macromolecules 39, 1724-1730 (2006) and Diaz et al., Journal of Polymer Science Part a-Polymer Chemistry 42, 4392-4403 (2004), one sequentially attaches MA-C6-HA12, Gly-MA, and MA-GRGDS to the macromer CTAs via RAFT (FIG. 21, Route 1) in the presence of 2,2′-azobis(isobutyronitrile) (AIBN) to give macromers 3 (m=10, 20), 4 (x=20, 40) and 5 (y=10), respectively. To achieve better control over the molecular weights and molecular weight distributions, the concentration of macromer CTAs are kept at 1 mM and the ratio of CTA to AIBN is kept at 80 to 1. One conducts the polymerization in N,N-dimethylformamide (DMF) or methanol/water at 65° C.

An alternative strategy towards the synthesis of functional macromer 5′ containing similar HA-nucleating domains, growth factor retention domains and cell adhesive domains is provided in FIG. 21 (route 2). Instead of directly grafting the highly polar peptide-containing methacrylamides to macromer CTAs, one grafts a less polar azido-containing methacrylamide MA-C3-N3. RAFT polymerization of less polar components results in higher overall yields and narrower molecule weight distributions. One conjugates AK5-HA12 and AK5-GRGDS to the azido domains using Cu(I)-catalyzed 1,3-dipolar cycloaddition, also known as “click” chemistry. Formation of the stable triazoles between azides and terminal alkynes may be done in the presence of other functional groups in aqueous or polar aprotic media. Polymers with azido or alkyne pendant side chains can both be prepared as “clickable” polymers. The use of acetylene-containing monomers in radical polymerizations, however, can be complicated by the undesired addition of the propagating radicals to the acetylene groups. Therefore, one avoids this complication by preparing “clickable” macromers containing the azido residues (macromer-N3) instead. One conjugates AK5-HA12 to the azido domain in DMF in the presence of CuBr (0.1 eq.) at room temperature. One carries out the attachment of the AK5-GRGDS motif to the more polar macromer 4′ in water, catalyzed by CuSO₄ (0.1 eq.) and sodium ascorbate (0.2 eq.) at room temperature. One grafts the AK5-GRGDS to the macromer, a final azido domain (10 repeats), after “clicking”, to allow for potential crosslinking of the macromers using the click chemistry. One compares the overall yield and polydispersity of macromer 5 vs. macromer 5′ at a selected domain length combination (n=20, m=10, x=20, y=10). One prepares grafts with varying copolymer chain length combinations (n=10, 20, 40; m=10, 20; x=20, 40 and y=10), crosslinking densities (8, 16 or 32 eq. cross-linker per macromer) and cross-linker lengths (MW 1 and 5 kD).

### Example XVIII

### Materials

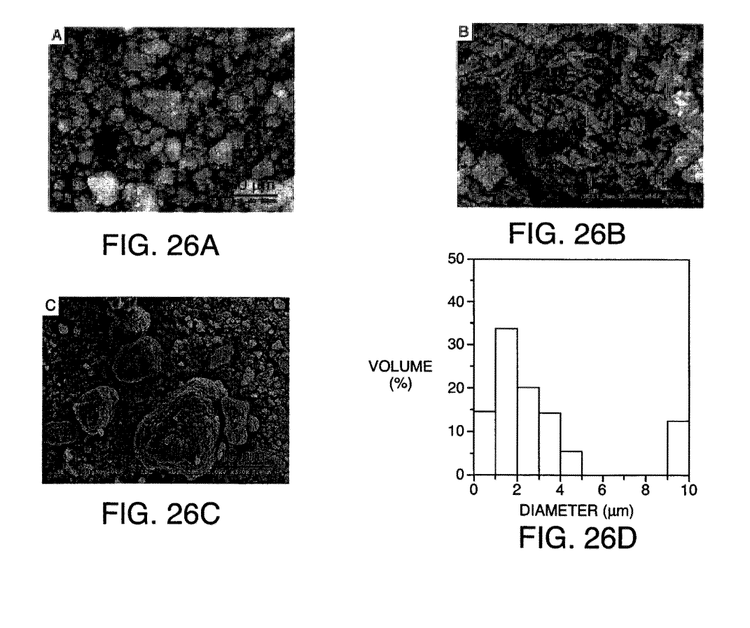

The radical inhibitors in the commercial HEMA and ethylene glycol dimethacrylate (EGDMA) from Aldrich (Milwaukee, Wis.) were removed via distillation under reduced pressure and by passing through a 4 Å molecular sieve column prior to use, respectively. Polycrystalline commercial HA powders (designated as ComHA) were purchased from Alfa Aesar (Ward Hill, Mass.) and used as received. The calcined HA powders (designated as CalHA) were obtained by treating ComHA at 1100° C. for 1 h. Prior to use, the CalHA powders were ground in a planetary agate mill for 2 h and then passed through a 38 μm sieve to remove larger agglomerates. The microstructures and size distributions of these HA particles are shown in FIG. 26. Cell culture media and supplements were purchased from Invitrogen (Carlsbad, Calif.) and the fetal bovine serum was purchased from HyClone (Logan, Utah). All reagents for histochemistry were purchased from Sigma (St Louis, Mo.).

### Preparation of Flexbone Composites

The HA content of the FlexBone is defined as the weight percentage of the HA incorporated over the total weight of the HA, monomer HEMA, and crosslinker EGDMA used in any given preparation. In a typical procedure, freshly distilled HEMA was mixed with EGDMA along with ethylene glycol (EG), water and aqueous radical initiators ammonium persulfate (I-1, 480 mg/mL) and sodium metasulfite (I-2, 180 mg/mL) at a volume ratio of HEMA:EGDMA:EG:I-1:I-2/100:2:35:20:5:5 (formulation 1). ComHA or CalHA powder was then added to the hydrogel mixture, thoroughly mixed by using a ceramic ball to break up the large agglomerates, and allowed to polymerize in a disposable syringe barrel or rigid PMMA tubing of a 7.0-mm or 4.7-mm inner diameter to afford composites with HA contents varying from 37 to 50%. The resulting elastic material was either used as it was (as-prepared), thoroughly exchanged with a large volume of water (fully hydrated), or freeze-dried. By altering the amount of EG and water relative to the HA, 70% of HA, a mineral content approximating that of human bone as provided for in An et al., Mechanical Testing of Bone and the Bone-Implant Interface, CRC Press, Boca Raton, Fla., pp. 41-63 (2000); and Phelps et al. Journal of Biomedical Material Research 51, 735-741 (2000), both of which are hereby incorporated by reference, can be used. For instance, a volume ratio of HEMA:EGDMA:EG:I-1:I-2/100:2:60:40:5:5 (formulation 2) was used to prepare composites containing 70% CalHA with consistent properties. (In this article, however, only properties of composites containing up to 50% HA are discussed.) The resulting composites are denoted as ComHA-N-# or CalHA-N-#, where N stands for the type of hydrogel formulation and # denotes the weight percentage of HA content. For instance, ComHA-1-50 represents FlexBone composite containing 50% commercial HA that is formed using crosslinking formulation 1. Unmineralized pHEMA control was prepared using formulation 1 in the absence of HA particles.

### Microstructural Characterization

The microstructures of the composites were characterized using environmental scanning electron microscopy (ESEM) on a Hitachi S-4300 SEN microscope (Hitachi, Japan). The chamber pressure was kept at approximately 35 Pa to avoid complete sample dehydration and surface charging during the observation. The chemical composition was analyzed using energy dispersive spectroscopy (EDS) (Noran System SIX, Thermoelectron, USA) attached to the ESEM.

### Mechanical Testing

To assess the compressive behavior of FlexBone in as-prepared, fully hydrated and freeze-dried states as a function of the mineral microstructure and content, unconfined compression tests were performed on two different instruments, a Q800 Dynamic Mechanical Analyzer (DMA) and a high capacity MTS, to accommodate the needs for high sensitivities and high loading capacities, respectively. All samples were tested in accordance with ASTM D695 with the exception of sample size and slenderness ratio (recommended ratio: 1:4 diameter-to-length) due to sample height limitation of the DMA instrument (≦5 mm) and the concern over the significant error that preparing and testing extremely small-diameter samples may introduce. Shorter cylinders were also used for the high capacity MTS due to the concern over sample buckling under extremely high compressive strains. All stress-strain curves presented are based on engineering stress and engineering strain recorded on each instruments, assuming a fixed cross-section of the material defined at the start of the test.

At least five specimens were tested for each sample. For as-prepared and water-equilibrated samples, cylindrical specimens with a diameter of 4.7 mm were transversely cut into 5.0-mm long cylinders using a custom-machined parallel cutter with adjustable spacing. Any visible roughness of the top and bottom surfaces of each specimen was reduced by sandpaper. An L-square was used to make sure that these surfaces were parallel prior to testing, and the final dimensions of each specimen were measured by a digital caliper. For freeze-dried samples, cylindrical specimens with the dimension of 7 mm×6 mm (diameter×height) were used.

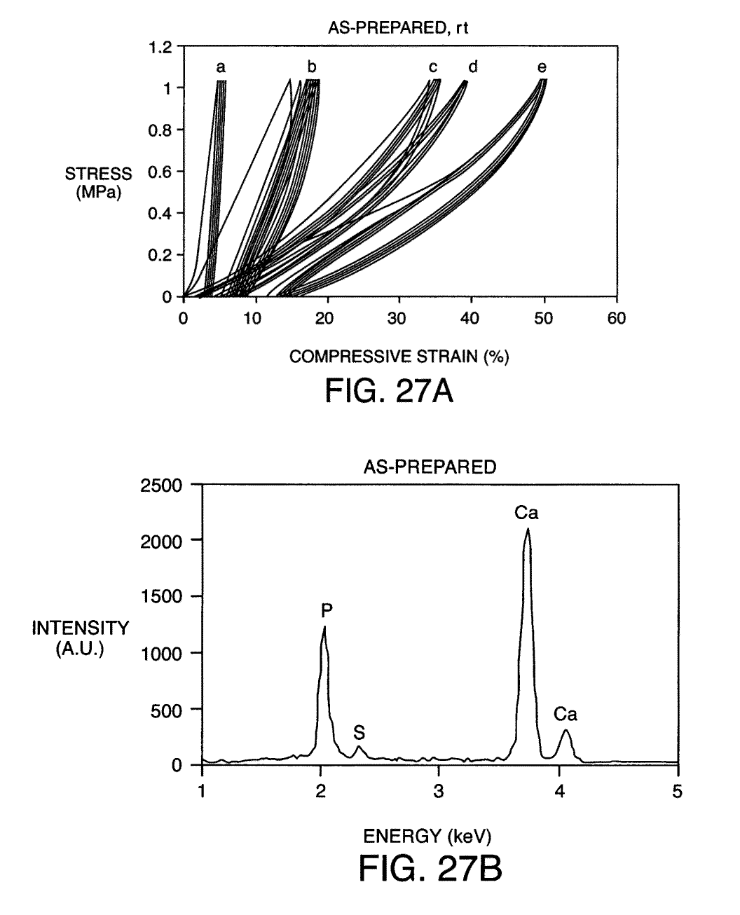

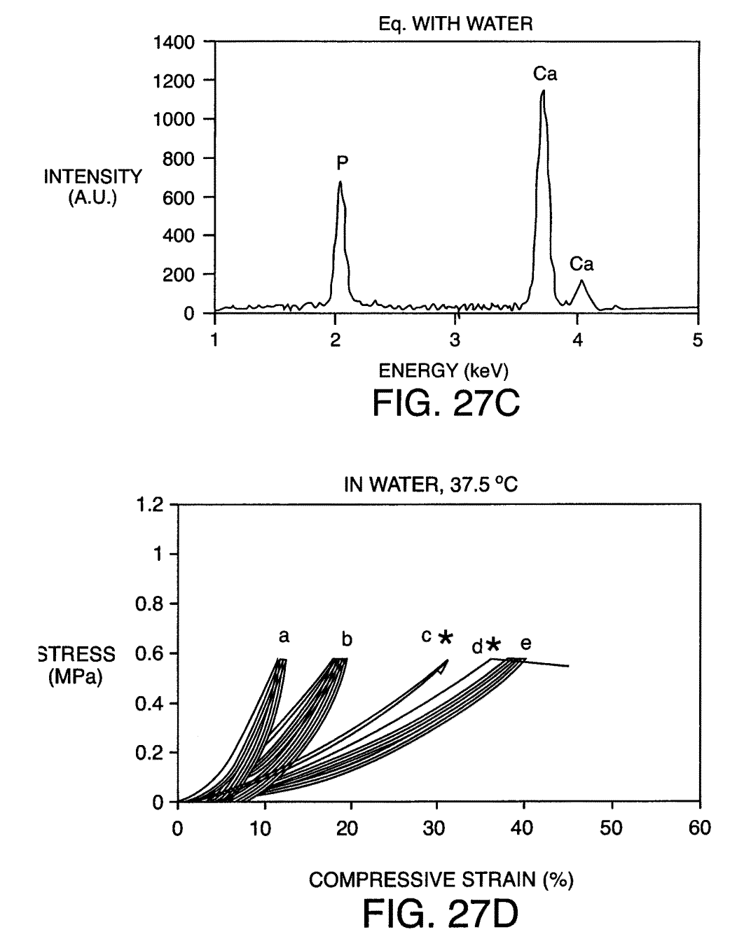

The compressive behavior of as-prepared and water-equilibrated FlexBone composites along with pHEMA control, particularly their elasticity, was evaluated on a Q800 DMA (TA Instruments) equipped with a submersion compression fixture. The instrument has an 18-N load cell, a force resolution of 10 μN and a displacement resolution of 1.0 nm. The as-prepared samples were compressed in a force-controlled mode in ambient air, ramping from 0.01 to 18.0 N at a rate of 3.0 N/min then back to 0.01 N at the same rate. The samples fully equilibrated with water were compressed in water at 37.5° C., ramping from 0.01 to 10.0 N at a rate of 3.0 N/min then back to 0.01 N at the same rate. To evaluate the reversibility of the compressive behavior, the controlled force cycle was repeated 10-40 times consecutively for each specimen unless the material failed (major cracks developed) during the force ramping, at which point the test would be terminated. In all cases, we observed little further shift of loading-unloading curves beyond 10 cycles. For clarity, in figures that compare the compressive behaviors among different samples (FIGS. 27, A and C), the first 10 cycles of the stress-strain curves from one representative specimen of each sample were plotted.

The compressive behavior of freeze-dried FlexBone composites, particularly their ability to withstand high compressive loads without exhibiting brittle fractures, was evaluated in ambient air on a high-capacity MTS servo-hydraulic mechanical testing machine (MTS Systems Corporation) equipped with a 100 kN load cell and stiff, non-deforming platens. The samples were loaded under displacement control at a rate of approximately 0.015 mm/s up to 80-90% compressive strain, while the corresponding loads and displacements were continuously monitored using the built-in load cell and linear variable displacement transducer (LVDT).

### Isolation and In Vitro Expansion of Rat BMSC

All animal procedures were conducted in accordance with the principles and procedures approved by the University of Massachusetts Medical School Animal Care and Use Committee. BMSC were isolated from long bones of 4-week old male Charles River SD strain rats as provided for in Miline et al., Endocrinology 139, 2527-2534 (1998), hereby incorporated by reference. Briefly, marrow was flushed from femur with a syringe containing MEM. After lysing red blood cells with sterile water, the marrow cells were centrifuged and resuspended in minimum essential medium (MEM) supplemented with 20% FBS, 0.2% penicillin-streptomycin and 1% L-glutamine, and passed through a sterile metal filter. Cells were expanded on tissue culture plates (10 million cells per 100-mm plate initial seeding density) with media changes on day 4 and every other day thereafter before they were lifted off for plating on FlexBone.

Subcutaneous Implantation of Flexbone Composite in Rats with and without Pre-Seeded BMSC

Thin half discs (7 mm in diameter, 1 mm in thickness) of FlexBone containing 40% ComHA (ComHA-1-40) were sterilized in 70% ethanol, re-equilibrated with sterile water before being seeded with BMSC and used for subcutaneous implantation in rats. Fifty microliters of BMSC suspension (in culture media described above) was loaded on the surface of thin disks of FlexBone to reach 5000-cells/cm² or 20,000-cells/cm² seeding density. The cell-seeded FlexBone were incubated at 37° C. in humidified environment with 5% CO₂ without additional media for 6 h to allow cell attachment to the FlexBone substrate. Additional media were then added and the cells were cultured on the substrates for two days before being used for implantation. Four sets of samples were used for each cell seeding treatment. Thin discs of FlexBone without preseeded BMSC were also used for implantation as controls.

Rats were anesthetized by intraperitoneal (IP) injection of ketamine/xylazine (50 mg/5 mg per kg). They were shaved and swabbed with betadine before two ¼ in bilateral skin incisions were made over the rib cage for insertion of the FlexBone discs with and without pre-seeded BMSC. The skin was closed with surgical staples and buprenorphine (0.02 mg/kg) was given subcutaneously. The rats were sacrificed by CO₂ inhalation and cervical dislocation at day 14 and day 28 for the retrieval of FlexBone. After removing the fibrous tissue encapsulation, the retrieved FlexBone was fixed in 4% paraformaldehyde (0.1 M phosphate buffer, pH 7.4) for 5 h at 4° C. before being analyzed by SEM, XRD, and histology.

### X-Ray Powder Diffraction

The crystalline phases of the mineral in the FlexBone composites before and after subcutaneous implantation in rats were evaluated by XRD with a Siemens D500 instrument using Cu Kα radiation. Phases were identified by matching the diffraction peaks to the JCPDS files.

### Histochemical Staining of Explanted Flexbone for Alkaline Phosphatase Activity

The 4% paraformaldehyde-fixed FlexBone explants were equilibrated in cacodylic buffer overnight, then in 30% sucrose solution (pH 7.3) for 2 days before being frozen-sectioned on a Bright Cryostat (Model OTF; Bright Instrument Ltd., Huntigdon, UK). Frozen-sectioning was repeated until reaching the depth of 100-200 μm away from the surface where the BMSC were initially seeded. The 12-μm frozen sections were held on adhesive slides using frozen sectioning tape. Histological staining for ALP activity, a marker of osteogenic differentiation, was performed as described in Drissi et al., Cancer Research 59, 3705-3711 (1999), incorporated herein by reference. Briefly, the frozen sections of FlexBone explants were incubated with 1.5 mM naphthol-As-Mx phosphate disodium salt, 0.1% Fast Red and 2.7% DMF (v/v) in 0.1 M Tris acid maleate buffer (pH 8.4) for 30 min, and the positive stains (in red) were detected by optical microscopy.

### Results

Preparation and Compressive Behavior of as-Prepared and Fully Hydrated Flexbone

FlexBone composites with varying mineral contents (37-70%) were prepared by crosslinking HEMA with 2% EGDMA in the presence of either porous aggregates of HA nanocrystals (ComHA) or compact micrometer-sized calcined HA (CalHA) particles (FIG. 26) using ethylene glycol as a solvent. Repetitive unconfined compressive tests performed on the as-prepared FlexBone with varying mineral contents revealed mineral content-dependent and mineral microstructure-dependent elastomeric compressive behavior. As indicated by the slopes of the compressive stress-strain curves shown in FIG. 27A, FlexBone composites are stiffer. (steeper slope) than the un-mineralized pHEMA hydrogel prepared with the same degree of crosslinking. In addition, FlexBone composites containing higher mineral contents are stiffer than those containing less HA particles of the same type, showing a positive correlation between the stiffness and the mineral content of the polymer-mineral composite. Notable difference in compressive behavior as a function of the type of HA components incorporated was also observed, with FlexBone containing ComHA much stiffer than those containing the same percentages of CalHA. Finally, good overlaps of stress-strain curves were observed when 10 consecutive compressive loading/unloading cycles up to approximately 1 MPa (the maximal applicable loads of the DMA instrument with the chosen sample size) were applied to all as-prepared composites. Such good recovery under compressive strains up to 40% depending on the composition is expected to facilitate the press-fitting of these composites into a defect area. As a reference, the peak contact stresses in natural human joints during light to moderate activity typically range from 0.5-6 MPa by most in vitro measurements as provided for in Ahmed et al., Journal of Biomechanical Engineering 105, 216-225 (1983); Brown et al., Journal of Biomechanics 16, 373-384 (1983); Whalen et al., Journal of Biomechanics 21, 825-837 (1988) and Brand et al., Iowa Orthopedic Journal 25, 82-94 (2005), all of which are hereby incorporated by reference, and up to 18 MPa by some in vivo measurements as provided for in Hodge et al., Proceedings of the National Academy of Sciences USA 83, 2879-2883 (1986) and Hodge et al., Journal of Bone and Joint Surgery 71, 1378-1386 (1989), both of which are incorporated by reference. Overall, our data suggest that as-prepared FlexBone exhibit excellent shape recovery under repetitive, physiologically relevant compressive stress despite their high (37-50%) mineral contents.

The as-prepared composites can undergo solvent exchange with water to give fully hydrated FlexBone. The residue sulfur-containing radical initiators trapped in the as-prepared composites could be removed during the wash with water as indicated by the disappearance of the S signal detected from the energy dispersive spectroscopy (EDS) performed on the cross-section of the composite upon equilibration with water as shown in FIG. 27B. The compressive behavior of fully hydrated FlexBone was examined at body temperature in water using a DMA equipped with a submersion compression fixture. As shown in FIG. 27C, mineral content-dependent and mineral microstructure-dependent compressive behavior similar to those exhibited by as-prepared FlexBone was observed with fully hydrated FlexBone. A noticeable difference, however, is that fully hydrated FlexBone containing CalHA failed (with major cracks formed) when >30% of compressive strain was applied. In contrast, FlexBone containing 37% and 50% ComHA could withstand repetitive megapascal compressive stress with excellent shape recovery in water. The difference observed with the hydrated composites containing same percentages of ComHA versus CalHA powders underscores the importance of the microstructures of the mineral component, and likely their differential behavior in interfacing with the polymer matrix, in determining the bulk mechanical properties of the polymer-mineral composites.

### Compressive Behavior and Micro-Structures of Freeze-Dried Flexbone

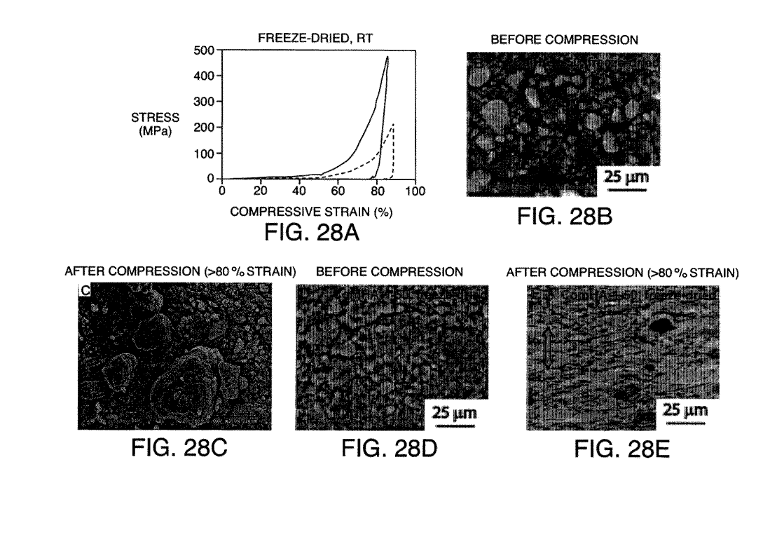

To better understand how the microstructure of the mineral component and the organic-inorganic interface dictates the macroscopic compressive behavior of FlexBone, we examined the microstructural response of freeze-dried composites containing ComHA versus CalHA under very high compressive stress and strains (>80%). Freeze-drying the fully hydrated FlexBone composites did not lead to the delamination of the evenly distributed mineral components, either ComHA or CalHA, from the polymer matrix that they were embedded in as shown in FIGS. 28, B and D. To apply high compressive strains to the freeze-dried composites, a high capacity MTS with 100 kN load cell was used to perform unconfined compression test. As expected, the freeze-dried composites were stiffer than their hydrated counterparts. Importantly, all tested freeze-dried FlexBone composites were able to withstand compressive stress in the order of hundreds of megapascals and compressive strains of >80% without exhibiting brittle fractures despite their high mineral contents as shown in FIG. 28 A. In contrast, PMMA-based bone/dental cements or poly(lactic acid)-HA composites reported in literature typically exhibited brittle fracture at 50-150 MPa compressive loading as provide for in Saha et al., Journal of Biomedical Material Research 18, 435-462 (1984); Shikinami et al., Biomaterials 20, 859-877 (1999) and Kuhn, Bone Cements, Springer, New York (2000), all of which are incorporated herein by reference.

A closer examination of the stress-strain curves revealed that freeze-dried composites containing ComHA tend to be stiffer than those containing same percentages of CalHA as shown in FIG. 28A. This is consistent with the trend observed with as-prepared and hydrated FlexBone under lower compressive stresses. SEM analysis of freeze-dried CalHA-1-50 after compression tests resulting in >80% strains revealed the formation of cracks within the hydrogel phase whereas no distortion or fracture of the micrometer-sized compact CalHA particles was observed (FIG. 28B vs. 28C). These cracks could affect the slope of the stress-strain curve. In contrast, the hydrogel-infiltrated aggregates of HA nanocrystals in freeze-dried ComHA-1-50 were flattened upon compression into plywood-like structures with no disruption of the continuity of the hydrogel matrix (FIG. 28D vs. 28E). The rearrangement of the nanometer-sized HA crystallites can provide a mechanism for energy dissipation within the composite under high compressive stresses.

### In Vivo Osteogenic Differentiation of BMSC Supported by Flexbone

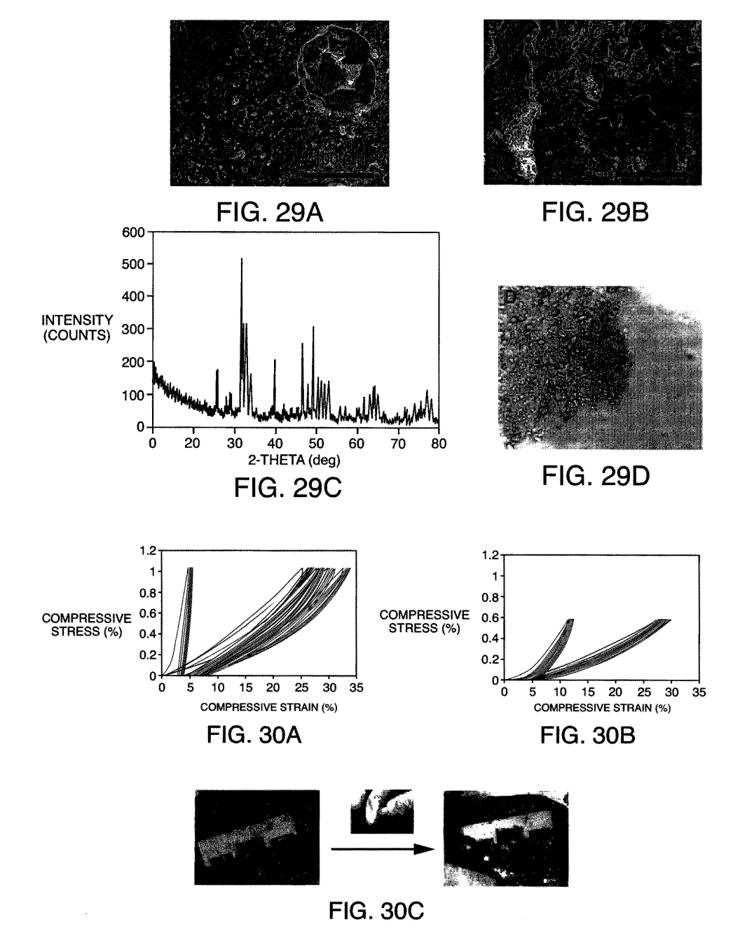

To test the cytocompatibility and the in vivo resorption of FlexBone, we seeded hydrated composites ComHA-1-40 with BMSC isolated from rat femur, and implanted them subcutaneously (SC) in 4-week old male Charles River SD strain rats. The composites were retrieved at 14 and 28 days, with a degree of fibrous tissue encapsulation observed in all cases. After removing the fibrous tissue, the morphology and mineral phase of the retrieved implant were examined by SEM and X-ray powder diffraction (XRD). Little macroscopic change in shape or size of the retrieved FlexBone was observed, reflecting the non-degradable nature of the hydrogel scaffold that defines the overall shape of the composite. However, surface roughening was observed with both 14- and 28-day explants regardless whether they were pre-seeded with BMSC prior to implantation (FIGS. 29 A and B). This is likely a combined outcome of slow dissolution of the mineral component and the extracellular matrix deposition from cells either pre-seeded on or newly attracted to the substrate in vivo. XRD analyses performed with the explanted composite (FIG. 29C) revealed a diffraction pattern matching with that of the ComHA powder, suggesting that the major mineral phase remained unchanged 4 weeks after the SC implantation.

To determine whether the composite can support the osteogenic differentiation of BMSC in vivo, the explanted composites with preseeded BMSC were stained histochemically for alkaline phosphatase (ALP) activity, a marker for osteogenic differentiation as disclosed in Vanhoof et al. Critical Reviews in Clinical Laboratory Science 31, 197-293 (1994), hereby incorporated by reference. To avoid the harsh paraffin embedding conditions that may compromise ALP enzymatic activity as provided for in Farley et al., Clinical Chemistry 39, 1878-1884 (1993), incorporated herein by reference, frozen sectioning was performed on the explants prior to ALP staining. As shown in FIG. 29D, ALP activity (indicated by red stains) was detected 14 days post-implantation on the periphery of the ComHA-1-40 preseeded with 5000-cells/cm² BMSC. More extensive ALP activity was also detected 28 days after the implantation on FlexBone pre-seeded with 20,000-cells/cm² BMSC. These data suggest that FlexBone was able to support the attachment and in vivo osteoblastic differentiation of osteoblast precursor cells.

### DISCUSSION

We report the preparation of a class of elastomeric pHEMA-HA composites, FlexBone, consisting of high percentages of osteoconductive HA using a straightforward protocol. The high viscosity of ethylene glycol, the solvent used during the fabrication of FlexBone, facilitated the easy dispersion of 50 wt % HA particles within the hydrogel formulation, thereby preventing the HA particles from settling by gravity during solidification. The intrinsic affinity of the hydroxyl side chains of the crosslinked pHEMA matrix to the surface of calcium apatite crystals led to the formation of strong interfaces between the organic and inorganic components. The good surface bonding of HA particles to the pHEMA matrix was maintained upon freeze-drying and contributed to the ability of the freeze-dried composites to withstand hundreds of megapascal compressive stress and >80% compressive strains without exhibiting brittle fractures.

Side-by-side comparisons of the compressive stress-strain curves obtained with FlexBone composites in as-prepared (FIG. 27A), hydrated (FIG. 27C) and freeze-dried (FIG. 28A) states revealed convincing correlations between the content/microstructures of the mineral component and the macroscopic compressive behavior of the composite. We have shown that the stiffness of FlexBone positively correlates with the content of a given microstructure of HA, with the slope of stress-strain curves of ComHA-1-50, for instance, steeper than that of ComHA-1-37 regardless of their solvent environment (ethylene glycol or water). The same trend was also observed with FlexBone containing CalHA. In natural bone, the bending, compression and tensile moduli of compact bone have been shown to exhibit a strong positive correlation with its mineral content as provided for in Follett et al., Bone 34, 783-789 (2004); Currey et al., Journal of Biomechanics 21, 131-139 (1988) and Currey et al., Journal of Biomechanics 23, 837-844 (1990), all of which are hereby incorporated by reference.

Our data have also demonstrated significant impact of the size and microscopic scale aggregation (structure) of HA minerals on the bulk compressive behavior of FlexBone. Whether in as-prepared, fully hydrated or freeze-dried state, FlexBone containing porous aggregates of HA nanocrystals (ComHA) are always significantly stiffer and stronger with respect to their resistance to crack formation and propagation under compression) than those containing the same percentage of compact micrometer-sized CalHA. The process of solvent exchange with water did not compromise the ability of as-prepared FlexBone containing ComHA to withstand repetitive physiological compressive stress and moderate (>10%) compressive strains, a feature highly desirable for the surgical insertion and use of FlexBone as synthetic bone grafts. In contrast, hydration significantly weakened the compressive strength of FlexBone containing CalHA (e.g. ultimate strength<0.6 MPa in water for CalHA-1-37 and CalHA-1-50), making them less suitable for moderate weight-bearing applications in vivo. Poor structural integration of polymer matrices with mineral components are also known to contribute to rapid and significant degradation of the mechanical integrity of other synthetic high mineral-content composites (e.g. PLA/HA composites) in aqueous environment as provided for in Russias et al., Material Science and Engineering C 26, 1289-1295 (2006), incorporated herein by reference.

We hypothesize that the sub-micrometer scale aggregation of HA nanoparticles in the ComHA acted as “sponges,” absorbing the prepolymer hydrogel formulation and yielding larger surface contact areas between the hydrogel matrix and the ComHA crystals. The better structural integration of the organic and inorganic components has translated into a significantly reduced tendency for crack formation and propagation within the resulting composites under high compressive stress. SEM studies further elucidated that the hydrogel-infiltrated spherical aggregates of HA nanocrystals flattened into plywood-like structures upon compression, providing an important energy-dissipation mechanism for FlexBone under compressive stress.

No simple extrapolation of earlier findings in ceramic, metallic, or intermetallic systems can predict the behavior of FlexBone since the combination of the soft hydrogel with the hard apatite crystals is quite unique. However, the microstructure-compressive behavior correlation revealed in our system is reminiscent of that observed with the analogous composite in nature-bone. It is well-known that the quality of the structural integration of the hard apatite crystals with the soft collagen network on nanoscopic and microscopic levels directly impact the mechanical properties of bone as provided for in Weiner et al., Annual Reviews of Material Science 28, 271-298 (1998), incorporated herein by reference. In fact, in aging bone, poorer structural integration of bone mineral with the collagen matrix is just as important as the decreasing mineral content in contributing to their weaker and more brittle mechanical properties. In the case of FlexBone, the impact of mineral microstructures on compressive behavior seems to have out-weighted that of the mineral content among the samples we examined. For instance, ComHA-1-37 is significantly stiffer than CalHA-1-50 and less likely to crack under megapascal-compressive stress in water (FIGS. 27 A and C).

Taken together, FlexBone containing ComHA exhibited tunable reversible compressive behavior in physiologically relevant environment (e.g. in water, at body temperature, and under megapascal compressive stress), making them appealing synthetic bone graft candidates. Subcutaneous implantation of ComHA-1-40 preseeded with BMSC in rats showed that the osteoconductive composite provided a cytocompatible environment to support the attachment, penetration, and osteogenic differentiation of BMSC in vivo. An ideal synthetic bone graft is designed to fill an area of defect to provide immediate structural stabilization and to expedite the healing and repair of the skeletal lesion. Ideally, the synthetic grafts can be eventually remodeled and replaced by newly synthesized bone. From this perspective, biodegradability and osteoinductivity of the synthetic bone grafts are just as important as their osteoconductivity, mechanical strength, and material handling characteristics (e.g. elasticity facilitating surgical insertion). Future improvements include engineering the biodegradability of the organic matrix, enhancing the in vivo dissolution rate of the osteoconductive mineral component to the mineral phase e.g. by introducing the more soluble β-tricalcium phosphate, β-TCP as provided for in Kwon et al., Journal of the American Ceramic Society 85, 3129-3131 (2002), hereby incorporated by reference, while locally retaining and releasing osteoinductive growth factors and cytokines on and from the synthetic scaffold.

### Conclusions

In summary, lightweight FlexBone composites containing high percentages of HA were prepared by crosslinking HEMA in the presence of HA using ethylene glycol as a solvent. Despite their high mineral contents (37-50%), the as-prepared composites exhibited elastomeric properties and reversible compressive behavior under moderate (megapascals) compressive stress. Owing to the excellent structural integration between the apatite mineral and the pHEMA network, freeze-dried FlexBone could withstand hundreds-of-megapascals compressive stress and >80% compressive strain without exhibiting brittle fractures (FIG. 28A). We further showed that the incorporation of porous aggregates of HA nanocrystals, rather than compact micrometer-sized calcined HA, into the hydrogel matrix could effectively improve the overall stiffness of FlexBone and its resistance to crack formation and propagation under compression. Upon equilibration with water, these composites retained good structural integration, and were able to support the attachment and osteoblastic differentiation of BMSC in vivo. Combined with the elasticity that facilitates the easy and stable surgical insertion of FlexBone into an area of bony defect and enables better accommodation to the micro movement of bone, these osteoconductive composites can find important orthopedic applications.

More broadly, the strong organic/inorganic interface achieved with FlexBone demonstrates that noncovalent binding between apatite crystals and a highly hydroxylated organic matrix can be exploited in the rational design of bone-like composites. In addition, the mineral content-dependent and mineral microstructure-dependent compressive behavior exhibited by FlexBone underlines the importance of taking into account the combined effect of these parameters in the rational design of functional structural composites.

### Example XIX

### Methods

### Materials.

The radical inhibitors in the commercial 2-hydroxyethyl methacrylate (HEMA, Aldrich) and ethylene glycol dimethacrylate (EGDMA, Aldrich) were removed via distillation under reduced pressure and by passing through a 4 Å molecular sieve column prior to use, respectively. Loose aggregates of polycrystalline hydroxyapatite nanocrystals (HA, Alfa Aesar, Ward Hill, Mass.) and β-tricalcium phosphate powders (TCP, Fluka) were used as received. Defined fetal bovine serum (FBS) was purchased from Hyclone, and recombinant proteins rhBMP-2/7 heterodimer and rmRANKL were purchased from R&D Systems (Minneapolis, Minn.) and reconstructed according to vendor instructions prior to use. Tetracycline hydrochloride (TCH, >95%) and all reagents for histochemistry were purchased from Sigma (St. Louis, Mo.).
Preparation of FlexBone and pHEMA with Varying Contents of TCH.
FlexBone composites containing between 0 and 5.0 wt % TCH were prepared using a protocol as described in Example XVIII. In a typical procedure, 0-5.0 wt % TCH was dissolved in the mixture of freshly distilled monomer HEMA, 2% cross-linker EGDMA and viscous solvent ethylene glycol under bath-sonication, before 25 wt % HA, 25 wt % TCP, and the aqueous radical initiators ammonium persulfate and sodium metasulfite were added and thoroughly mixed (Table I). The pasty mixture was immediately drawn into a rigid acrylic tubing (United States Plastic Corp., pre-washed with ethanol to remove radical inhibitors and air-dried prior to use) of an inner diameter of ⅛″ (3.2 mm) or 3/16″ (4.8 mm), and allowed to solidify at room temperature overnight. The resulting elastic material was either used as it was for antibiotic release kinetics study and E. coli inhibition assay, or thoroughly exchanged with a large volume of water for 24 h (to remove ethylene glycol and residue unpolymerized monomer and radical initiators) for subsequent mechanical testing and cell culture study.

### Mechanical Testing.

The compressive behavior of FlexBone in fully hydrated state as a function of TCH content was analyzed using a Q800 Dynamic Mechanical Analyzer (DMA, TA Instruments) equipped with a submersion compression fixture. The instrument has an 18-N load cell, a force resolution of 10 μN and a displacement resolution of 1.0 nm. All samples were tested in accordance with ASTM D695 with the exception of sample size and slenderness ratio due to sample height limitation of the DMA instrument (≦5 mm) and the concern over the significant error that preparing and testing extremely small-diameter samples may introduce. Three cylindrical specimens (Φ=4.8 mm; H=5.0 mm) were tested for each sample. An L-square was used to make sure that the sanded top and bottom surfaces were parallel prior to testing, and the final dimensions of each specimen were measured by a digital caliper. The as-prepared samples were compressed in a force-controlled mode in water at 37.0° C., increasing from 0.03 N to 10.0 N at a rate of 3.0 N/min then reduced to 0.03 N at the same rate. The fully hydrated samples were compressed in a force-controlled mode in water at 37.0° C., increasing from 0.03 N to 10.0 N at a rate of 3.0 N/min then reduced to 0.03 N at the same rate. The controlled force cycle was repeated 10 times consecutively for each specimen. All stress-strain curves presented are based on the engineering stress and engineering strain recorded, assuming a fixed cross-section of the material defined at the start of the test.
TCH Release Kinetics from FlexBone Vs. from pHEMA.
TCH has strong optical absorptions at the UV-Vis region, enabling the characterization of its release kinetics by spectroscopy as disclosed in He et al., Journal of Macromolecular Science B 45, 515-524 (2006) and Kenawy et al., Journal of Controlled Release 81, 57-64 (2002), both of which are incorporated by reference. The release of TCH from FlexBone vs. pHEMA hydrogel in water as a function of time and the initial TCH incorporation was monitored over 1 week at 357.9 nm. Each freshly prepared sample (Φ=4.8 mm; H=5.0 mm) was placed in MilliQ water at a 100:1 water-to-sample mass ratio without agitation for 30 min, 1 h, 2 h, 4 h, 8 h, 16 h, 28 h, 52 h, 76 h, 100 h, 148 h, and 172 h, respectively. The release kinetics was determined by quantifying the TCH released into water at various time points. A standard absorption-TCH concentration curve was generated by preparing and measuring the absorption of TCH standards (100 mM, 1.0 mM, 100 μM, 50.0 μM, 25.0 μM, 10.0 μM, 5.0 μM, 2.0 μM, 1.0 μM, and 0.5 μM) at 357.9 nm. Percentage of TCH release from FlexBone or pHEMA was plotted over time for each composition examined.
Antibiotic Activities of the TCH Released from FlexBone or Phema.
The antibiotic activity of the TCH released from FlexBone or pHEMA was evaluated by its ability to inhibit E. coli culture. Warm LB (25 g/L)-Agar (15 g/L) solution was poured into P-150 cell culture dishes (35 mL/plate) and cooled to room temperature. The surface of the LB-Agar plates were coated with 250 μL E. coli XL-2 solution (OD_{600 nm}=0.256) with glass beads and cultured at 37° C. for 10 min before thin discs (Φ=4.8 mm, H=2.0 mm) of FlexBone graft containing 5.0 wt % TCH were placed on the surface (six discs per plate). The E. coli culture was continued at 37.0° C. and the diameters of the clear zones developed surrounding the discs were monitored at 80 min, 160 min, 4 h, 8 h, 16 h, 21 h, 24 h, 28 h, 32 h, 40 h, and 48 h, respectively. Three specimens were examined for each time point. The diameters of the clear zones (average±s.d.) as a function of time are plotted.

### Equilibrium Water Content (EWC) and the Loading of Recombinant Proteins.

Three specimens of each water-equilibrated FlexBone sample and pHEMA control (Φ=3.2 mm; H=5.0 mm) were weighed before and after being freeze-dried. EWC is calculated using the following formula: EWC=[(hydrated weight−dry weight)/dry weight]×100%. The average EWC's for FlexBone and pHEMA were determined as 37.99±0.64% and 50.16±0.69%, respectively. The maximal aqueous loading volume (Vₘₐₓ) of each pre-weighed freeze-dried FlexBone or pHEMA specimen is determined as Vₘₐₓ (μL)=[EWC×dry weight (mg)]/(1 mg/μL). Recombinant protein rhBMP-2/7 was reconstructed according to the manufacturer's instruction, and the protein solution was applied to freeze-dried FlexBone in the pre-determined maximal aqueous loading volume (Vₘₐₓ) to yield the final loading dose of 20 ng/graft. Recombinant protein rmRANKL was loaded in a similar fashion to both freeze-dried FlexBone and freeze-dried pHEMA control to reach a 10 ng/graft final loading dose.
Osteogenic Trans-Differentiation of Murine Myoblast C2C12 Cells Induced by the rhBMP-2/7 Locally Released from FlexBone.
The bioactivity of the exogenous rhBMP-2/7 released from FlexBone was evaluated by its ability to induce osteogenic trans-differentiation of mouse myoblast C2C12 cells three days after placing the FlexBone graft pre-loaded with rhBMP-2/7 in the low mitogen C2C12 culture. C2C12 cells were seeded at 5,000/cm² in a 24-well plate in DMEM (0.5 mL/well) supplemented with 10% FBS and 1% Pen-Strep, and allowed to attach overnight. Upon cell attachment (day 1), the culture media were switched to low mitogen DMEM (0.5 mL/well) supplemented with 5% FBS and 1% Pen-Strep, and a FlexBone graft freshly loaded with 20-ng rhBMP-2/7 was added to each well (N=3). The culture was continued for three days without further media change. In the positive control wells, 20-ng rhBMP-2/7 was supplemented directly in the low mitogen media (40-ng/mL) without a FlexBone carrier on day one. Cells were fixed on day 3 by 4% paraformaldehyde (in PBS, pH 7.4), and stained for alkaline phosphatase (ALP), a marker of osteogenic differentiation, in 0.1 M Tris acid maleate buffer (pH 8.4) containing 1.5 mM naphthol-As-Mx phosphate disodium salt, 0.1% Fast Red and 2.7% DMF (v/v) for 30 min as provided for in Drissi et al., Cancer Research 59, 3705-3711 (1999), incorporated herein by reference.
Osteoclastic Differentiation of Murine Macrophage RAW264.7 Cells Induced by the rmRANKL Released from FlexBone.
The bioactivity of the exogenous rmRANKL released from FlexBone is evaluated by its ability to induce osteoclastic differentiation of murine macrophage RAW264.7 cells six days after placing the FlexBone graft pre-loaded with rnRANKL in the RAW264.7 culture. RAW264.7 cells were seeded at 10,000/cm² in a 24-well plate in alpha-MEM (0.5 mL/well) supplemented with 10% FBS and 1% Pen-Strep, and allowed to attach overnight. One FlexBone or pHEMA graft freshly loaded with 10-ng rmRANKL was then added to each well (N=3 for each combination) on day one, and the culture was continued for 6 days with media change every two days without additional supplement of rmRANKL. In the positive control well, 10 ng rmRANKL was supplemented directly in the culture media without a graft carrier every two days. In the negative control well, 10 ng rmRANKL was supplemented directly in the culture media without a graft carrier on day one, and the medium was changed every two days without any additional supplement of rmRANKL. The culture was terminated on day six when the formation of multinucleated cells was observed in the positive control well as well as in those containing the FlexBone grafts. Grafts were removed from all wells before the cells were stained for tartrate-resistant acid phosphatase (TRAP) activities using the Sigma TRAP kit following the manufacturer's instructions.

### Results and Discussion

### Preparation, Compressive Behavior and Microstructures of FlexBone Containing 25 wt % HA-25 wt % TCP and 0-5.0 wt % TCH.

To promote the in vivo dissolution of the mineral component of FlexBone, TCP, a biomineral that is known to have faster in vitro dissolution rate than HA as disclosed in Kwon et al., Journal of the American Ceramic Society 85, 3129-3131 (2002), incorporated herein by reference, was incorporated along with the loose aggregates of nanocrystalline HA within the pHEMA matrix. Specifically, FlexBone composites containing a fixed mineral content of 25 wt % HA-25 wt % TCP and varying contents (0-5.0 wt %) of TCH were prepared (Table I). The procedure involves crosslinking HEMA with 2% EGDMA in the presence of solubilized TCH and a mixture of loose aggregates of nanocrystalline HA and the more compact TCP particles dispersed in viscous ethylene glycol. Our previous study showed that the incorporation of nanometer-sized HA rather than compact micrometer sized HA could lead to better integrated structural composites (by virtually maximizing the hydrogel-HA interfacial contact area) that were more resistant to fracture formation and propagation as disclosed herein. Thus, it is important to ensure that the incorporation of the denser TCP particles in FlexBone would not significantly compromise its ability to withstand repetitive moderate compressive stress, a property necessary for its stable press-fitting into a critical size bony defect.

Unconfined compression tests performed at 37° C. revealed that FlexBone containing 25 wt % HA/25 wt % TCP was less stiff than that containing 50 wt % HA in both as-prepared and hydrated states, as indicated by the slopes of the stress-strain curves (FIGS. 30A and 30B, dark blue vs. green curves). This observation was consistent with our previous findings that FlexBone containing loose aggregates of nanometer-sized HA tend to be stiffer than that containing the same weight percentage of more compact calcined HA powders as disclosed herein. It is important to note, however, the TCP-containing FlexBone was still able to withstand >10 consecutive moderate compressive loading/unloading cycles without fracturing when as much as half of the nanometer-sized HA was replaced by the more compact TCP. Specifically, under the maximal compressive stress applied (>1 MPa for as-prepared sample and 0.6 MPa for hydrated sample), the TCP-containing FlexBone was able to recover from up to 30% repetitive compressive strains, suggesting that it had maintained the desired elastomeric and fracture-resistant surgical handling characteristics. Indeed, as shown in FIG. 30C, a piece of fully hydrated FlexBone containing 25 wt % HA-25 wt % TCP was readily press-fitted into a 5-mm segmental defect in rat femur.

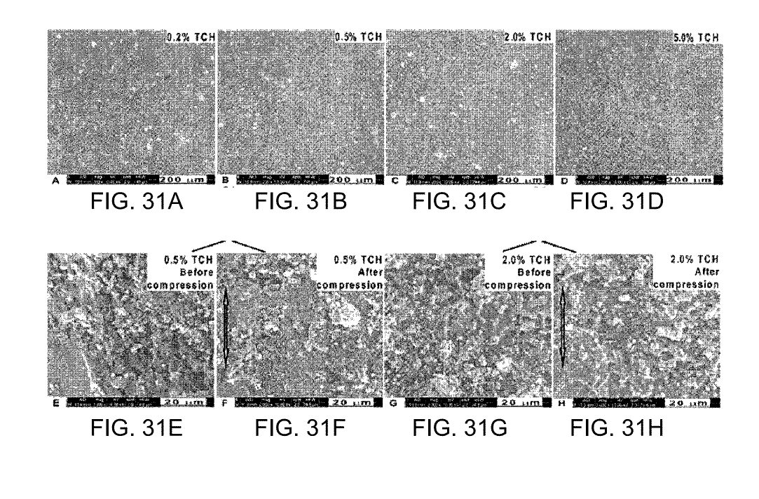

Unconfined compression tests and SEM were also performed to investigate the impact of the encapsulation of TCH on the compressive behavior and microstructures of FlexBone. Whereas the stiffness (slope of the stress-strain curves) of as prepared FlexBone fluctuated as TCH contents varied from 0.1 wt % to 5.0 wt % (FIG. 30A), no substantial difference in stress-strain curves of water-equilibrated composites was detected (FIG. 30B). Good overlaps were observed not only among the 10 consecutive compressive loading/unloading (up to 0.6 MPa) curves for each hydrated sample but also across samples containing varying amounts (0, 0.5 wt %, 2.0 wt %, and 5.0 wt %) of TCH. This observation suggests that the TCH tightly bound to the HA/TCP matrix (those retained after the 24-h equilibration with water) had minimal impact on the compressive behavior of the composite. SEM micrographs confirmed that the incorporation of up to 5.0 wt % TCH within FlexBone did not alter the distribution of the mineral components within the elastic pHEMA matrix (FIGS. 31A-31D). In addition, the microstructures of all as-prepared composites fully recovered after being subjected to >10 consecutive 1-MPa compressive loading/unloading cycles irrespective of their TCH contents (FIGS. 31E-31H), supporting the underlying excellent structural integration of the HA/TCP component with the elastic polymer matrix.

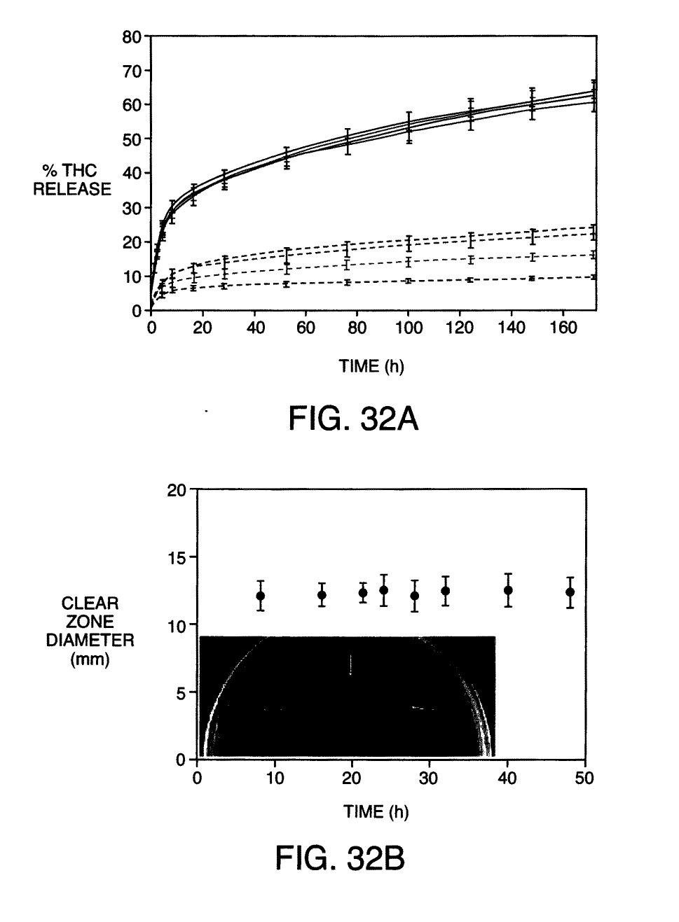

In Vitro Release of TCH from FlexBone Vs. pHEMA.
To explore the use of FlexBone composites as synthetic bone grafts for the repair of critical-sized bony defect with minimal risk for infections, the ability to encapsulate antibiotics and release them in a sustained and dosed-dependent manner is desired. Tetracyclines are broad-spectrum antibiotics that are also known for their non-antimicrobial capacity to reduce pathological bone resorption via MMP inhibition as disclosed in Greenwald et al., Bone 22, 33-38 (1998); Williams et al., Inhibition of Matrix Metalloproteinases: Therapeutic Applications, 191-200 (1999) and Holmes et al., Bone 35, 471-478 (2004), all of which are incorporated herein by reference, and promote bone formation as disclosed in Golub et al., Research Communications in Chemical Pathology and Pharmacology 68, 27-40 (1990); Williams et al., Bone 19, 637-644 (1996); Sasaki et al., Calcified Tissue International 50, 411-419 (1992); Sasaki et al., Anatomical Record 231, 25-34 (1991); Bain et al., Bone 21, 147-153 (1997) and Gomes et al., Acta Biomaterialia 4, 630-637 (2008), all of which are hereby incorporated by reference. The in vitro release of TCH from FlexBone vs. pHEMA hydrogel in water as a function of time and initial TCH incorporation was monitored by visible spectroscopy at 357.9 nm over one week. As shown in FIG. 32A, FlexBone released TCH in a sustained and dose-dependent manner, achieving ˜10% and ˜20% release in 7 days from composites containing 0.5 wt % and 5.0 wt % TCH, respectively. In contrast, un-mineralized pHEMA hydrogel quickly released 30% of TCH in the first 8 hours, and reaching >60% release of TCH by day seven, irrespective of their initial TCH contents. The substantially slower and dose-dependent release of TCH from FlexBone is presumably due to the strong chelating interaction between TCH and the calcified matrix of FlexBone. The antibiotic activity of the TCH released from FlexBone was examined by its ability to inhibit E. coli culture. As shown in FIG. 32B, the TCH released from FlexBone inhibited E. coli culture as indicated by the formation of the clear zones surrounding the grafts placed over the surface of the E. coli agar plate by 8 hours. The clear zones were sustained throughout the two-day-old bacterial culture.
Localized Release of rhBMP-2/7 from FlexBone Induces Osteogenic Trans-Differentiation of C2C12 Cells in Culture.

To augment the healing capacity of critical-sized bony defects, we propose to engineer the biochemical microenvironment of FlexBone to achieve localized and sustained delivery of growth factors and cytokines promoting osteointegration and graft remodeling to the site of a defect. BMP-2 is required for the initiation of fracture healing as disclosed in Tsuji et al., Nature Genetics 38, 1424-1429 (2006), incorporated herein by reference, and has been clinically used as an adjuvant for spinal fusion and fracture union. BMP-2/7 heterodimer, known for its more potent osteogenicity than either BMP-2 or BMP-7 homodimer as provided for in Zhu et al., Journal of Bone and Mineral Research 19, 2021-2032 (2004) and Laflamme et al., Biomedical Materials 3 (2008), both of which are hereby incorporated by reference, is chosen as an osteogenic component to promote the osteointegration of FlexBone upon implantation to a site of skeletal defect. To examine the in vitro release characteristics of rhBMP-2/7 from FlexBone and guide the dose selection for subsequent in vivo studies, we utilized the well-documented BMP-2-induced osteogenic trans-differentiation of C2C12, a mouse skeletal muscle cell line, as a cell culture model as provided for in Katagiri et al., Journal of Cell Biology 127, 1755-1766 (1994), hereby incorporated by reference.

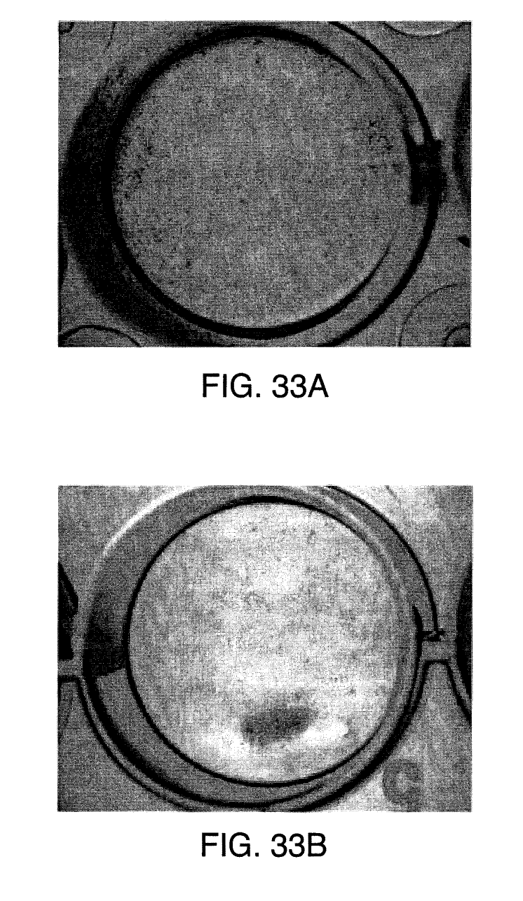

As a positive control, we first showed that a single dose of 40-ng/mL rhBMP-2/7 supplemented directly to the C2C12 culture without a graft carrier was able to induce osteogenic trans-differentiation of C2C12 as indicated by the detection of ALP activity (red stains) across the culture plate by day three (FIG. 33A). This dose is significantly lower than that required for BMP-2-induced osteogenic trans-differentiation of C2C12 at a similar cell seeding density as provided for in Katagiri et al., Journal of Cell Biology 127, 1755-1766 (1994) supporting the more potent osteogenic property of the BMP-2/7 heterodimer. When the same dose of rhBMP-2/7 was pre-absorbed on a FlexBone carrier before being placed in the C2C12 culture, the osteoblastic trans-differentiation of C2C12 cells was only detected in a highly confined region surrounding the FlexBone graft (FIG. 33B). This observation suggests that the osteogenic property of rhBMP-2/7 was retained upon its release from FlexBone while the release of rhBMP-2/7 from FlexBone was achieved in a highly localized fashion, a property desired for scaffold-based local therapy.

Sustained Release of rmRANKL from FlexBone Induces Osteoclast Differentiation of RAW264.7 Cells in Culture.

RANKL regulates osteoclastic bone resorption during skeletal repair and bone graft remodeling as disclosed in Ito et al., Nature Medicine 11, 291-297 (2005) and Kon et al., Journal of Bone and Mineral Research 16, 1004-1014 (2001), both of which are hereby incorporated by reference. Osteoclasts are hematopoietically derived, multinucleated cells that arise from the monocyte/macrophage lineage as provided fo rin Ash et al., Nature 283, 669-670 (1980), incorporated herein by reference. It is known that RANKL, which is expressed on both stromal cells and osteoblasts, plays an essential role in the regulation of osteoclast differentiation as provided for in Hsu et al., Proceedings of the National Academy of Sciences USA 96, 3540-3545 (1999); Yasuda et al., Proceedings of the National Academy of Sciences USA 95, 3597-3602 (1998) and Lacey et al., Cell 93, 165-176 (1998), all of which are incorporated by reference. Soluble recombinant form of RANKL was found sufficient in the induction of osteoclast differentiation from macrophage in in vitro cultures. To explore the potential of modulating the remodeling of FlexBone in vivo by the delivery of exogenous recombinant RANKL protein, we investigated in this study whether the release of rmRANKL from FlexBone can be achieved in a sustained manner within a physiologically relevant time frame. We choose RANKL-induced osteoclast differentiation of murine macrophage cells RAW264.7 as a cell culture model for this investigation. RAW 264.7 cells are known to express high levels of RANK mRNA as provided for in Hsu et al., Proceedings of the National Academy of Sciences USA 96, 3540-3545 (1999) and can be differentiated into osteoclasts upon the induction of RANKL.

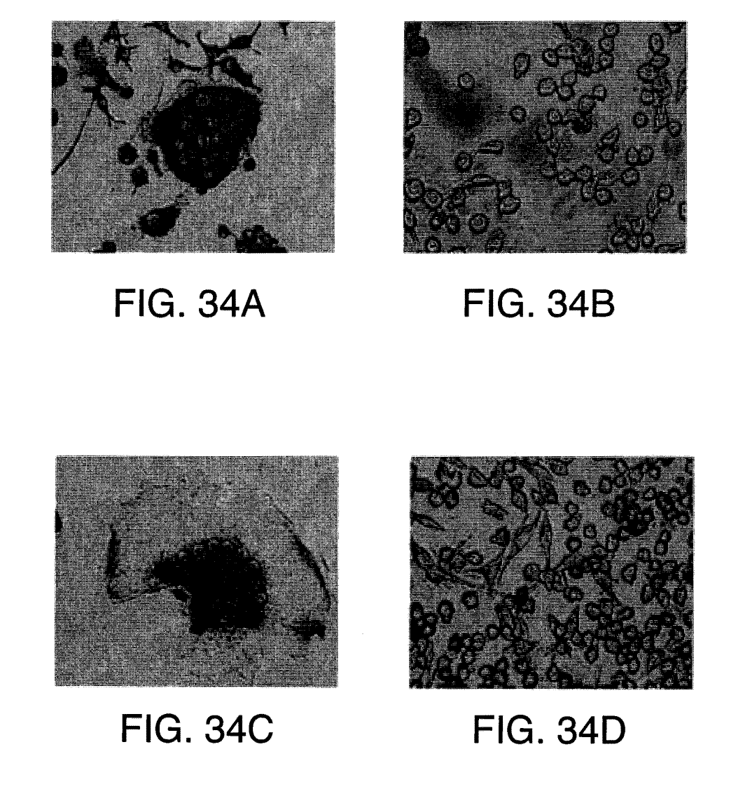

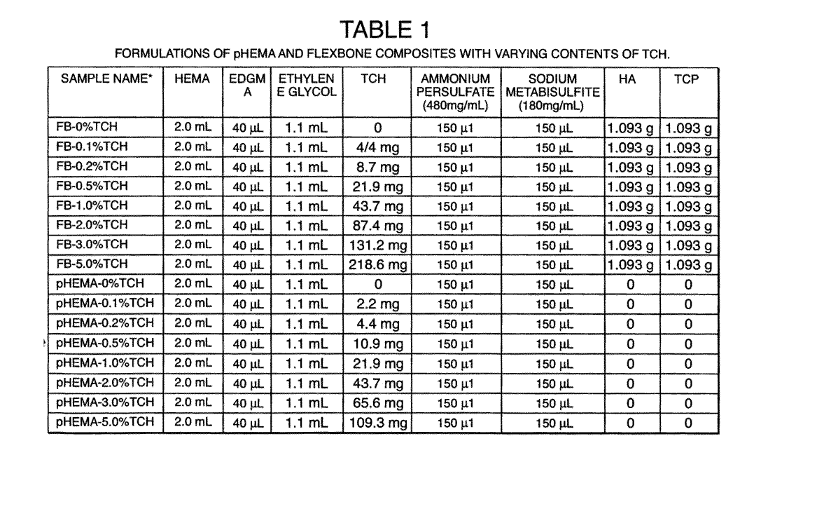

To effectively induce the osteoclast differentiation of RAW264.7 in culture, continued supplementation of a sufficient amount of RANKL is required. As shown by the control experiments, a single supplement of 10-ng rmRANKL directly to the RAW267.4 culture was not sufficient in inducing the osteoclast differentiation (FIG. 34D) while the continued supplement of 10-ng rmRANKL every other day led to the formation of TRAP-positive multinucleated osteoclasts by day six (FIG. 34C). When the FlexBone graft pre-absorbed with 10-ng rmRANKL was placed in culture, however, osteoclast differentiation of RAW264.7 was observed by day six without any additional supplement of rmRANKL (media changed every other day (FIG. 34A)). This observation suggests that FlexBone was able to release rmRANKL in a sustained manner over six days. In contrast, when the un-mineralized pHEMA hydrogel pre-absorbed with the same amount of rmRANKL was placed in the culture, no osteoclastic differentiation of RAW264.7 was observed by day six (FIG. 34B), likely due to the rapid burst release of the RANKL from the hydrogel matrix. These results suggest that the HA/TCP component of FlexBone, integrated within the hydrogel matrix, played an important role in achieving the balance between sequestering and releasing the recombinant protein.

### CONCLUSIONS

Synthetic bone grafts that possess the structural and biochemical microenvironment emulating that of natural bone and exhibit good surgical handling characteristics are highly desired in orthopedic care yet challenging to design and fabricate. Bone is a natural organic-inorganic structural composite. The mineral component of bone (its content, its structural integration with the organic matrices, and its affinity for a wide range of matrix proteins and soluble factors) plays an important role in defining the structural, mechanical and biochemical properties of the calcified tissue as disclosed in Follet et al., Bone 34, 783-789 (2004); Tong et al., Calcified Tissue International 72, 592-598 (2003); Gilbert et al., Journal of Biological Chemistry 275, 16213-16218 (2000) and Stubbs et al., Journal of Bone and Mineral Research 12, 1210-1222, all of which are hereby incorporated by reference. While not limiting the present invention to any particularly theory, it is believed that synthetic structural composite containing a high percentage of osteoconductive biominerals that are structurally well-integrated with an organic polymer matrix can be engineered to provide both the structural and biochemical framework of a viable synthetic bone graft.

FlexBone is a structural composite consisting of a high content of osteoconductive HA/TCP minerals (50 wt %) that are well dispersed and integrated within an elastomeric crosslinked pHEMA hydrogel network. The combination of elasticity and high osteoconductive mineral content of FlexBone, coupled with its ability to withstand repetitive moderate compressive loadings in an aqueous environment at physiological temperature, makes this structural composite uniquely suited as a synthetic bone substitute candidate for the repair of critical-sized skeletal defects. We have demonstrated in this study that the biochemical and therapeutic (antibiotic) microenvironment promoting the remodeling of bone grafts and reducing the risk for infections can be conveniently integrated with FlexBone without compromising its mechanical and structural integrity. The release of the antibiotic TCH and exogenous recombinant proteins rhBMP-2/7 and rmRANKL pre-encapsulated with FlexBone was achieved in a localized and sustained manner over one week, a time frame within which the effects of these molecules on inhibiting infection and promoting early osteointegration and graft healing are most significant as disclosed in Bourque et al., Laboratory Animal Science 42, 369-374 (1992); Raiche et al., Journal of Biomedical Materials Research Part A 69A, 342-350 (2004); Macey et al., Journal of Bone and Joint Surgery-American 71A, 722-733 (1989) and Pufe et al., Cell and Tissue Research 309, 387-392 (2002), all of which are hereby incorporated by reference. The minimal loading doses of these biomolecules determined in the cell culture study also provide a rational starting point for the subsequent evaluation of the in vivo performance of FlexBone with and without exogenous growth factors using a rat femoral segmental defect model. Using the straightforward small molecule encapsulation and growth factor loading methods we developed, a wide range of therapeutic agents and signaling molecules can be integrated with FlexBone. This provides an exciting opportunity to utilize the elastic osteoconductive composite bone graft to augment the biochemical microenvironment of hard-to-heal bony defects resulting from aging, cancer, trauma or metabolic diseases, contributing to the more effective surgical treatment of these debilitating conditions.

1-15. (canceled)

16. A method for preparing an elastic composite material loaded with cells or biomolecules, comprising:
a) providing an elastic composite material comprising:
i) a polymer matrix comprising polymethacrylate and ethylene glycol units, wherein the polymethacrylate has side chains comprising hydroxyl groups, and
ii) hydroxyapatite aggregates distributed within said polymer matrix, wherein said composite material is between 10% and 70% by weight hydroxyapatite aggregates within said polymer matrix, wherein said hydroxyapatite aggregates have sizes between 50 nanometers and 50 micrometers;
b) creating pores in said composite material providing an elastic composite material; and
c) mixing said elastic composite material with a component selected from the group consisting of cells and biomolecules providing an elastic composite loaded with said component.

17. The method of claim 16, wherein said cells are bone marrow cells.

18. The method of claim 17, wherein said biomolecule is selected from the group consisting of growth factors, cytokines, and gene vectors.

19. The method of claim 18, wherein said subject is a selected from the group consisting of a human, mouse, rat, dog, cat, rabbit, pig, horse, and ape.

20-26. (canceled)

27. The method of claim 16, wherein the elastic composite material is porous.

28. The method of claim 16, wherein the cells are bone marrow stem cells.

29. The method of claim 28, wherein the bone marrow stem cells are mesenchymal stem cells.

30. The method of claim 16, wherein the biomolecule is selected from a recombinant protein or a gene vector encoding the protein.

31. The method of claim 16, wherein the biomolecule is a growth factor.

32. The method of claim 16, wherein the biomolecule is a recombinant bone morphogenetic protein selected from the group consisting of BMP-2 and BMP-2/7.

33. The method of claim 27, wherein the porous elastic composite material retains reversible compressive behavior under a compressive load of 2.6 MPa strains up to 40% when hydrated, and is capable of withstanding a compression force between 150 and 500 MPa without brittle fracture when freeze-dried.

34. The method of claim 16, wherein the polymethacrylate is poly(2-hydroxyethyl methacrylate).

35. The method of claim 16, wherein the composite material is capable of withstanding up to 700 MPa compressive load and up to 90% compressive strain without exhibiting brittle fracture when freeze-dried.

36. The method of claim 16, wherein said hydroxyapatite is polycrystalline.

37. The method of claim 16, further comprising:
d) implanting said loaded composite into a subject.
